(19) United States
(12) Patent Application Publication
(10) Pub. No.: US 2009/0069317 A1
(43) Pub. Date: Mar. 12, 2009

(54) Insecticidal Methods Using 3-Amino-1,2-Benzisothiazole Derivatives

(75) Inventors: Matthias Pohlman, Heidelberg (DE); Wolfgang von Deyn, Neustadt (DE); Florian Kaiser, Mannheim (DE); Michael Rack, Eppelheim (DE); Henricus Maria Martinus Bastiaans, Usingen (DE); Douglas D. Anspaugh, Apex, NC (US); Deborah L. Culbertson, Fuquay Varina, NC (US); Henry Van Tuyl Cotter, Raleigh, NC (US)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/094,342

(22) PCT Filed: Nov. 15, 2006
(86) PCT No.: PCT/EP2006/068469

§ 371 Date: May 20, 2008

Related U.S. Application Data
(60) Provisional application No. 60/738,443, filed on Nov. 21, 2005.

(51) Int. Cl. C07D 275/04; A01N 43/80; A01N 43/84; C07D 413/12; A01P 7/02; A01P 7/04; A01P 5/00

(52) U.S. Cl. 514/233.8; 514/373; 548/209; 544/135

(57) ABSTRACT

The invention relates to insecticidal methods using 3-amino-1,2-benzoisothiazole compounds of the formula (I) wherein the variables n and R¹ to R⁶ are as defined in the description. The invention relates to methods of combating or controlling insects, arachnids or nematodes, to methods for protecting growing plants from attack or infestation by insects, arachnids or nematodes, and to methods for the protection of seeds from soil insects and of the seedlings' roots and shoots from soil and foliar insects.

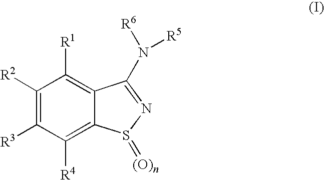

The present invention relates to the method of combating or controlling insects, arachnids or nematodes comprising contacting an insect, arachnid or nermatode or their food supply, habitat or breeding grounds with an pesticidally effective amount of at least one compound of formula I or a composition comprising at least one compound of formula I and a method for the protection of seeds from soil insects and of the seedlings' roots and shoots from soil and foliar insects comprising contacting the seeds before sowing and/or after pregermination with 3-amino-1,2-benzisothiazole compounds of the formula I or composition comprising at least one compound of formula I:

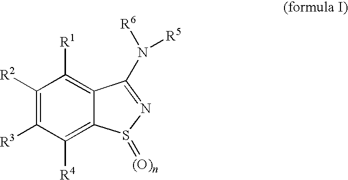

wherein
n is 0, 1 or 2;
R¹ is hydrogen, nitro, cyano, azido, amino, halogen, sulfonylamino, sulfenylamino, sulfinylamino, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, C₁-C₆-alkylthio, (C₁-C₆-alkyl)amino, di(C₁-C₆-alkyl)amino, C₁-C₆-alkylsulfinyl or C₁-C₆-alkylsulfonyl, wherein the eleven last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy, and wherein
   R^{1a} is selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl or heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ or N—Rⁿ, wherein Rⁿ is hydrogen, C₁-C₆-alkyl or (C₁-C₆-alkyl)-carbonyl;
   R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, (C₁-C₄-alkoxy)carbonyl, amino, (C₁-C₄-alkyl)amino, di(C₁-C₄-alkyl)amino, aminocarbonyl, (C₁-C₄-alkyl)aminocarbonyl, di(C₁-C₄-alkyl)aminocarbonyl, sulfonyl, sulfonylamino, sulfenylamino, sulfanylamino and C(═O)—R^{2a} or C(═O)—R^{3a} or C(═O)—R^{4a}, and wherein R^{2a} or R^{3a} or R^{4a} are selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl or heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ or N—Rⁿ, wherein Rⁿ is hydrogen, C₁-C₆-alkyl or (C₁-C₆-alkyl)-carbonyl;
R⁵ is selected from the group consisting of OR^{5a}, C₁-C₁₀-alkyl, C₂-C₁₀-alkenyl, C₂-C₁₀-alkinyl, C₃-C₁₀-cycloalkyl, wherein the four last-mentioned radicals may be unsubstituted, partially or fully halogenated and/or may carry 1-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1-3 substituents selected from the group consisting of C₁-C₆-alkyl, C₁-C₆-haloalkyl, C₁-C₆-alkoxy and C₁-C₆-haloalkoxy and wherein
   R^{5a} is selected from hydrogen, C₁-C₁₀-alkyl, C₁-C₁₀-acyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl and wherein the carbon atoms of the radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
R⁶ is selected from the group consisting of C₁-C₁₀-alkyl, C₂-C₁₀-alkenyl, C₂-C₁₀-alkinyl, C₃-C₁₀-cycloalkyl, wherein these radicals may be unsubstituted, partially or fully halogenated and/or may carry 1-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1-3 substituents selected from the group consisting of C₁-C₆-alkyl, C₁-C₆-haloalkyl, C₁-C₆-alkoxy, C₁-C₆-haloalkoxy or
R⁵/R^{5a} and R⁶ can join to form a 3-10 membered ring, optionally substituted and optionally containing, in addition to optionally substituted C₁-C₅-alkyl, independently 1 to 3 N, NRⁿ, O, S, SO, SO₂ moieties, which can be optionally unsaturated, and wherein Rⁿ is hydrogen, C₁-C₆-alkyl or (C₁-C₆-alkyl)-carbonyl;
   or the enantiomers or diastereomers, salts or esters thereof.

In spite of the commercial insecticides, acaricides and nematicides available today, damage to crops, both growing and harvested, caused by insects, arachnids and nematodes still occurs. Therefore, there is a continuing need to develop new insecticidal, acaricidal and nematicidal agents.

It was therefore an object of the present invention to provide new pesticidal compositions, new compounds and new methods for the control of insects, arachnids or nematodes and of protecting growing plants from attack or infestation by insects, arachnids or nematodes and also new methods for the protection of seeds from soil insects and of the seedlings' roots and shoots from soil and foliar insects.

We have found that these objects are achieved by the compounds of formula I.

Depending on the substitution pattern, the compounds of formula I can contain one or more chiral centers, in which case they are present as enantiomer or diastereomer mixtures. Subject matter of this invention are not only compositions containing these mixtures but also those containing the pure enantiomers or diastereomers.

Some compounds of formula I have been described inter alia in DE 1915387, WO 03/87072, JP 06220030, DE 1670920 and DE 1545842. However, an insecticidal, acaricidal or nematicidal activity of compounds of formula I have not been disclosed yet.

Amino- and amino-acylated 1,2-benzisothiazole compounds have been described by Drabek for an insecticidal activity in EP 207891, EP 191734, DE 3544436, EP 138762, EP 133418 and EP 110829, or 3-Amidinobenzisothiazole 1,1-dioxides for same use in EP 86748. JP 01319467 describes the preparation of N-acylated amino-benzisothiazoles and their 1,2-dioxyde derivatives for insecticidal purposes. Sulfonyl compounds and aphicidal compositions based on monosubstituted 3-amino-1,2-benzisothiazole-1,1-dioxyde derivatives have been described in EP 0033984.

The problem underlying the present invention was the need of new compounds for insecticidal use, having an excellent efficacy and a good chemical and physico-chemical stability.

This problem is solved by the present application by the use of N,N-disubstituted 3-amino-1,2 benzisothiazole compounds as insecticidal compounds. They showed a particular better stability then those derivatives used in prior art, and have a better insectidal activity.

In this specification and in the claims, reference will be made to a number of terms that shall be defined to have the following meanings:

“Salt” as used herein includes adducts of compounds I with maleic acid, dimaleic acid, fumaric acid, difumaric acid, methane sulfenic acid, methane sulfonic acid, and succinic acid. Moreover, included as “salts” are those that can form with, for example, amines, metals, alkaline earth metal bases or quaternary ammonium bases, including zwitterions. Suitable metal and alkaline earth metal hydroxides as salt formers include the salts of barium, aluminum, nickel, copper, manganese, cobalt zinc, iron, silver, lithium, sodium, potassium, magnesium or calcium. Additional salt formers include chloride, sulfate, acetate, carbonate, hydride, and hydroxide. Desirable salts include adducts of compounds I with maleic acid, dimaleic acid, fumaric acid, difumaric acid, and methane sulfonic acid.

“Halogen” will be taken to mean fluoro, chloro, bromo and iodo.

The term “alkyl” as used herein refers to a branched or unbranched saturated hydrocarbon group having 1 to 6, 1 to 8 or 1 to 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “haloalkyl” as used herein refers to a straight-chain or branched alkyl groups having 1 to 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and heptafluorpropyl.

“Alkylamino” refers to a straight-chain or branched alkyl group having 1 to 6 carbon atoms (as mentioned above) which is bonded through a nitrogen linkage.

Similarly, “alkoxy”, “(alkyl)carbonyl”, “alkylsulfinyl”, “alkylsulfonyl”, “alkylthio” or “alkylamino” refer to straight-chain or branched alkyl groups having 1 to 6 or 1 to 8 or 1 to 10 carbon atoms (as mentioned above) bonded through oxygen or sulfur linkages, respectively, at any bond in the alkyl group. Examples include methoxy, ethoxy, propoxy, isopropoxy, methylthio, ethylthio, propylthio, isopropylthio, and n-butylthio.

The term “alkenyl” as used herein intends a branched or unbranched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” as used herein refers to a branched or unbranched unsaturated hydrocarbon group containing at least one triple bond, such as ethynyl, propynyl, 1-butynyl, 2-butynyl, and the like.

Aryl: mono- or bicyclic 5- to 10-membered aromatic ringsystem, e.g. phenyl or naphthyl.

Hetaryl: a 5- to 10-membered heteroaromatic ring system containing 1 to 4 heteroatoms selected from oxygen, sulfur and nitrogen, e.g. 5-membered hetaryl, containing 1 to 4 nitrogen atoms, such as pyrrolyl, pyrazolyl, imidazolyl, triazolyl, and tetrazolyl; or

5-membered hetaryl, containing 1 to 4 nitrogen atoms or 1 to 3 nitrogen atoms and 1 sulfur or oxygen atom, e.g. furyl, thienyl, pyrrolyl, isoxazolyl, isothiazolyl, pyrazolyl, oxazolyl, thiazolyl, imidazolyl, oxadiazolyl, thiadiazolyl, oxadiazolyl, triazolyl, and tetrazolyl; or
5-membered hetaryl, containing 1 to 4 nitrogen atoms or 1 to 3 nitrogen atoms and 1 sulfur or oxygen atom, in which two adjacent ring carbon atoms or one nitrogen atom and an adjacent carbon atom can be bridged by buta-1,3-dien-1,4-diyl; or 6-membered hetaryl, containing 1 to 4 nitrogen atoms or 1 to 3 nitrogen atoms and 1 sulfur or oxygen atom, e.g. 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

A saturated or partially saturated mono- or bicyclic 5- to 10-membered ringsystem containing 1 to 3 heteroatoms selected from nitrogen and oxygen intends e.g. a saturated monocyclic 5- to 7-membered ringsystem containing 1 to 3 heteroatoms selected from nitrogen and oxygen, such as pyridine, pyrimidine, pyrrolidine, piperazine, homopiperazine, morpholine, and piperidine; or e.g. a saturated bicyclic 7- to 10-membered ringsystem containing 1 to 3 heteroatoms selected from nitrogen and oxygen, such as 1,4-diazabicyclo[4.3.0]nonane, 2,5-diazabicyclo[2.2.2]octane, and 2,5-diazabicyclo[2.2.1]heptane.

Cycloalkyl: monocyclic 3- to 6-, 8-, 10- or 12-membered saturated carbon atom rings, e.g. C₃-C₈-cycloalkyl such as cyclopropyl, cyclobutyl, cyclopentyl, cyclohexyl, cycloheptyl and cyclooctyl.

With respect to the intended use of the compounds of formula I, particular preference is given to the following meanings of the substituents, in each case on their own or in combination:

Preference is also given to compounds of formula I wherein R¹ is halogen, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy, C₁-C₄-haloalkoxy, (C₁-C₄-alkoxy)carbonyl and C₃-C₈-cycloalkyl and wherein R^{1a} is selected from the group consisting of hydrogen, hydroxy, C₁-C₆-alkoxy, C₁-C₆-haloalkoxy, amino, (C₁-C₆)-alkyl-amino, di-(C₁-C₆)-alkyl-amino, C₁-C₆-alkyl, aryl and aryl-C₁-C₆-alkyl.

Particular preference is given to compounds of formula I wherein R¹ is halogen, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy and C₁-C₄-haloalkoxy.

Also, particular preference is given to compounds of formula I wherein R¹ is halogen, preferably fluoro or chloro, most preferably chloro.

Furthermore, particular preference is given to compounds of formula I wherein R¹ is C₁-C₆-alkoxy or C₁-C₆-haloalkoxy.

Preference is given to compounds of formula I wherein R², R³ and R⁴ is hydrogen.

Preference is given to compounds of formula I wherein R⁵ and R⁶ are C₁-C₆-alkyl or C₂-C₆-alkynyl, in particular C₁-C₄-alkyl.

Preference is also given to compounds of formula I wherein n is 2. Preference is also given to compounds of formula I wherein n is 0. Particular preference is given to compounds of formula I wherein n is 2.

With respect to their use, particular preference is given to compounds of formula I compiled in the tables below.

Moreover, the groups mentioned for a substituent in the tables are on their own, independently of the combination in which they are mentioned, a particularly preferred embodiment of the respective substituents in question.

Compounds of formula Ia

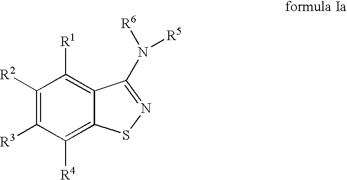

Preferred are compounds of formula Ia, wherein R¹, R², R³ and R⁴ corresponds in each case to a row of Tables 1 to 178 in combination with R⁵ and R⁶ accordingly in each case to a row of Tables A1 to A 444.

Compounds of formula Ib:

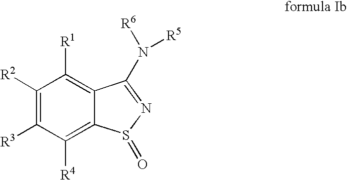

Preferred are compounds of formula Ib, wherein R¹, R², R³ and R⁴ corresponds in each case to a row of Tables 179 to 356 in combination with R⁵ and R⁶ accordingly in each case to a row of Tables A1 to A 444.

Formula Ic:

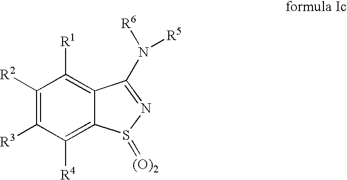

Preferred are compounds of formula Ic, wherein R¹, R², R³ and R⁴ corresponds in each case to a row of Tables 357 to 534 in combination with R⁵ and R⁶ accordingly in each case to a row of Tables A1 to A 444.

These preferred examples are for use in methods of combating or controlling insects, arachnids or nematodes comprising contacting an insect, arachnid or nematode or their food supply, habitat or breeding grounds with an pesticidally effective amount of at least one compound of formulae (Ia), (Ib) or (Ic) or a composition comprising at least one compound of those formulae as defined herein. They are also for use in methods for the protection of seeds from soil insects and of the seedlings' roots and shoots from soil and foliar insects comprising contacting the seeds before sowing and/or after pregermination with at least one compounds of the formulae (Ia), (Ib) or (Ic) or composition comprising at least one compound of those formulae as defined herein. Some of the individual compounds will be defined more than once in different rows of the tables regardless of any preference.
Table 1: Compounds of formula Ia, wherein each of R³ and R⁴ are H, R² is H, R¹ is CH₃ and wherein R⁵ and R⁶ are as defined in one row of table A;
Table 2: Compounds of formula Ia, wherein each of R³ and R⁴ are H, R² is H, R¹ is C₂H₅ and wherein R⁵ and R⁶ are as defined in one row of table A;
Table 3: Compounds of formula Ia, wherein each of R³ and R⁴ are H, R² is H, R¹ is CF₃ and wherein R⁵ and R⁶ are as defined in one row of table A;
Table 4: Compounds of formula Ia, wherein each of R³ and R⁴ are H, R² is H, R¹ is F and wherein R⁵ and R⁶ are as defined in one row of table A;
Table 5: Compounds of formula Ia, wherein each of R³ and R⁴ are H, R² is H, R¹ is Cl and wherein R⁵ and R⁶ are as defined in one row of table A;
Table 6: Compounds of formula Ia, wherein each of R³ and R⁴ are H, R² is H, R¹ is Br and wherein R⁵ and R⁶ are as defined in one row of table A;
Table 7: Compounds of formula Ia, wherein each of R³ and R⁴ are H, R² is H, R¹ is J and wherein R⁵ and R⁶ are as defined in one row of table A;
Table 8: Compounds of formula Ia, wherein each of R³ and R⁴ are H, R² is H, R¹ is OCH₃ and wherein R⁵ and R⁶ are as defined in one row of table A;
Table 9: Compounds of formula Ia, wherein each of R³ and R⁴ are H, R² is H, R¹ is OC₂H₅ and wherein R⁵ and R⁶ are as defined in one row of table A;
Table 10: Compounds of formula Ia, wherein each of R³ and R⁴ are H, R² is H, R¹ is OCF₃ and wherein R⁵ and R⁶ are as defined in one row of table A;
Table 11: Compounds of formula Ia, wherein each of R³ and R⁴ are H, R² is H, R¹ is OCHF₂ and wherein R⁵ and R⁶ are as defined in one row of table A;
Table 12: Compounds of formula Ia, wherein each of R³ and R⁴ are H, R² is H, R¹ is OCClF₂ and wherein R⁵ and R⁶ are as defined in one row of table A;
Table 13: Compounds of formula Ia, wherein each of R³ and R⁴ are H, R² is F, R¹ is CH₃ and wherein R⁵ and R⁶ are as defined in one row of table A;
Table 14: Compounds of formula Ia, wherein each of R³ and R⁴ are H, R² is F, R¹ is C₂H₅ and wherein R⁵ and R⁶ are as defined in one row of table A;
Table 15: Compounds of formula Ia, wherein each of R³ and R⁴ are H, R² is F, R¹ is CF₃ and wherein R⁵ and R⁶ are as defined in one row of table A;
Table 16: Compounds of formula Ia, wherein each of R³ and R⁴ are H, R² is F, R¹ is F and wherein R⁵ and R⁶ are as defined in one row of table A;
Table 17: Compounds of formula Ia, wherein each of R³ and R⁴ are H, R² is F, R¹ is Cl and wherein R⁵ and R⁶ are as defined in one row of table A;
Table 18: Compounds of formula Ia, wherein each of R³ and R⁴ are H, R² is F, R¹ is Br and wherein R⁵ and R⁶ are as defined in one row of table A;
Table 19: Compounds of formula Ia, wherein each of R³ and R⁴ are H, R² is F, R¹ is J and wherein R⁵ and R⁶ are as defined in one row of table A;
Table 20: Compounds of formula Ia, wherein each of R³ and R⁴ are H, R² is F, R¹ is OCH₃ and wherein R⁵ and R⁶ are as defined in one row of table A;
Table 21: Compounds of formula Ia, wherein each of R³ and R⁴ are H, R² is F, R¹ is OC₂H₅ and wherein R⁵ and R⁶ are as defined in one row of table A;
Table 22: Compounds of formula Ia, wherein each of R³ and R⁴ are H, R² is F, R¹ is OCF₃ and wherein R⁵ and R⁶ are as defined in one row of table A;
Table 23: Compounds of formula Ia, wherein each of R³ and R⁴ are H, R² is F, R¹ is OCHF₂ and wherein R⁵ and R⁶ are as defined in one row of table A;
Table 24: Compounds of formula Ia, wherein each of R³ and R⁴ are H, R² is F, R¹ is OCClF₂ and wherein R⁵ and R⁶ are as defined in one row of table A;
Table 25: Compounds of formula Ia, wherein each of R³ and R⁴ are H, R² is Cl, R¹ is CH₃ and wherein R⁵ and R⁶ are as defined in one row of table A;
Table 26: Compounds of formula Ia, wherein each of R³ and R⁴ are H, R² is Cl, R¹ is C₂H₅ and wherein R⁵ and R⁶ are as defined in one row of table A;
Table 27: Compounds of formula Ia, wherein each of R³ and R⁴ are H, R² is Cl, R¹ is CF₃ and wherein R⁵ and R⁶ are as defined in one row of table A;
Table 28: Compounds of formula Ia, wherein each of R³ and R⁴ are H, R² is Cl, R¹ is F and wherein R⁵ and R⁶ are as defined in one row of table A;
Table 29: Compounds of formula Ia, wherein each of R³ and R⁴ are H, R² is Cl, R¹ is Cl and wherein R⁵ and R⁶ are as defined in one row of table A;
Table 30: Compounds of formula Ia, wherein each of R³ and R⁴ are H, R² is Cl, R¹ is Br and wherein R⁵ and R⁶ are as defined in one row of table A;
Table 31: Compounds of formula Ia, wherein each of R³ and R⁴ are H, R² is Cl, R¹ is J and wherein R⁵ and R⁶ are as defined in one row of table A;
Table 32: Compounds of formula Ia, wherein each of R³ and R⁴ are H, R² is Cl, R¹ is OCH₃ and wherein R⁵ and R⁶ are as defined in one row of table A;
Table 33: Compounds of formula Ia, wherein each of R³ and R⁴ are H, R² is Cl, R¹ is OC₂H₅ and wherein R⁵ and R⁶ are as defined in one row of table A;
Table 34: Compounds of formula Ia, wherein each of R³ and R⁴ are H, R² is Cl, R¹ is OCF₃ and wherein R⁵ and R⁶ are as defined in one row of table A;
Table 35: Compounds of formula Ia, wherein each of R³ and R⁴ are H, R² is Cl, R¹ is OCHF₂ and wherein R⁵ and R⁶ are as defined in one row of table A;
Table 36: Compounds of formula Ia, wherein each of R³ and R⁴ are H, R² is Cl, R¹ is OCClF₂ and wherein R⁵ and R⁶ are as defined in one row of table A;
Table 37: Compounds of formula Ia, wherein each of R³ and R⁴ are H, R² is Br, R¹ is CH₃ and wherein R⁵ and R⁶ are as defined in one row of table A;
Table 38: Compounds of formula Ia, wherein each of R³ and R⁴ are H, R² is Br, R¹ is C₂H₅ and wherein R⁵ and R⁶ are as defined in one row of table A;
Table 39: Compounds of formula Ia, wherein each of R³ and R⁴ are H, R² is Br, R¹ is CF₃ and wherein R⁵ and R⁶ are as defined in one row of table A;
Table 40: Compounds of formula Ia, wherein each of R³ and R⁴ are H, R² is Br, R¹ is F and wherein R⁵ and R⁶ are as defined in one row of table A;
Table 41: Compounds of formula Ia, wherein each of R³ and R⁴ are H, R² is Br, R¹ is Cl and wherein R⁵ and R⁶ are as defined in one row of table A;
Table 42: Compounds of formula Ia, wherein each of R³ and R⁴ are H, R² is Br, R¹ is Br and wherein R⁵ and R⁶ are as defined in one row of table A;
Table 43: Compounds of formula Ia, wherein each of R³ and R⁴ are H, R² is Br, R¹ is J and wherein R⁵ and R⁶ are as defined in one row of table A;
Table 44: Compounds of formula Ia, wherein each of R³ and R⁴ are H, R² is Br, R¹ is OCH₃ and wherein R⁵ and R⁶ are as defined in one row of table A;
Table 45: Compounds of formula Ia, wherein each of R³ and R⁴ are H, R² is Br, R¹ is OC₂H₅ and wherein R⁵ and R⁶ are as defined in one row of table A;
Table 46: Compounds of formula Ia, wherein each of R³ and R⁴ are H, R² is Br, R¹ is OCF₃ and wherein R⁵ and R⁶ are as defined in one row of table A;
Table 47: Compounds of formula Ia, wherein each of R³ and R⁴ are H, R² is Br, R¹ is OCHF₂ and wherein R⁵ and R⁶ are as defined in one row of table A;
Table 48: Compounds of formula Ia, wherein each of R³ and R⁴ are H, R² is Br, R¹ is OCClF₂ and wherein R⁵ and R⁶ are as defined in one row of table A;
Table 49: Compounds of formula Ia, wherein each of R³ and R⁴ are H, R² is J, R¹ is CH₃ and wherein R⁵ and R⁶ are as defined in one row of table A;
Table 50: Compounds of formula Ia, wherein each of R³ and R⁴ are H, R² is J, R¹ is C₂H₅ and wherein R⁵ and R⁶ are as defined in one row of table A;
Table 51: Compounds of formula Ia, wherein each of R³ and R⁴ are H, R² is J, R¹ is CF₃ and wherein R⁵ and R⁶ are as defined in one row of table A;
Table 52: Compounds of formula Ia, wherein each of R³ and R⁴ are H, R² is J, R¹ is F and wherein R⁵ and R⁶ are as defined in one row of table A;
Table 53: Compounds of formula Ia, wherein each of R³ and R⁴ are H, R² is J, R¹ is Cl and wherein R⁵ and R⁶ are as defined in one row of table A;
Table 54: Compounds of formula Ia, wherein each of R³ and R⁴ are H, R² is J, R¹ is Br and wherein R⁵ and R⁶ are as defined in one row of table A;
Table 55: Compounds of formula Ia, wherein each of R³ and R⁴ are H, R² is J, R¹ is J and wherein R⁵ and R⁶ are as defined in one row of table A;
Table 56: Compounds of formula Ia, wherein each of R³ and R⁴ are H, R² is J, R¹ is OCH₃ and wherein R⁵ and R⁶ are as defined in one row of table A;
Table 57: Compounds of formula Ia, wherein each of R³ and R⁴ are H, R² is J, R¹ is OC₂H₅ and wherein R⁵ and R⁶ are as defined in one row of table A;
Table 58: Compounds of formula Ia, wherein each of R³ and R⁴ are H, R² is J, R¹ is OCF₃ and wherein R⁵ and R⁶ are as defined in one row of table A;
Table 59: Compounds of formula Ia, wherein each of R³ and R⁴ are H, R² is J, R¹ is OCHF₂ and wherein R⁵ and R⁶ are as defined in one row of table A;
Table 60: Compounds of formula Ia, wherein each of R³ and R⁴ are H, R² is J, R¹ is OCClF₂ and wherein R⁵ and R⁶ are as defined in one row of table A;
Table 61: Compounds of formula Ia, wherein each of R² and R⁴ are H, R³ is H, R¹ is C₂H₅ and wherein R⁵ and R⁶ are as defined in one row of table A;
Table 62: Compounds of formula Ia, wherein each of R² and R⁴ are H, R³ is H, R¹ is CF₃ and wherein R⁵ and R⁶ are as defined in one row of table A;
Table 63: Compounds of formula Ia, wherein each of R² and R⁴ are H, R³ is H, R¹ is F and wherein R⁵ and R⁶ are as defined in one row of table A;
Table 64: Compounds of formula Ia, wherein each of R² and R⁴ are H, R³ is H, R¹ is Cl and wherein R⁵ and R⁶ are as defined in one row of table A;
Table 65: Compounds of formula Ia, wherein each of R² and R⁴ are H, R³ is H, R¹ is Br and wherein R⁵ and R⁶ are as defined in one row of table A;
Table 66: Compounds of formula Ia, wherein each of R² and R⁴ are H, R³ is H, R¹ is J and wherein R⁵ and R⁶ are as defined in one row of table A;
Table 67: Compounds of formula Ia, wherein each of R² and R⁴ are H, R³ is H, R¹ is OCH₃ and wherein R⁵ and R⁶ are as defined in one row of table A;
Table 68: Compounds of formula Ia, wherein each of R² and R⁴ are H, R³ is H, R¹ is OC₂H₅ and wherein R⁵ and R⁶ are as defined in one row of table A;
Table 69: Compounds of formula Ia, wherein each of R² and R⁴ are H, R³ is H, R¹ is OCF₃ and wherein R⁵ and R⁶ are as defined in one row of table A;
Table 70: Compounds of formula Ia, wherein each of R² and R⁴ are H, R³ is H, R¹ is OCHF₂ and wherein R⁵ and R⁶ are as defined in one row of table A;
Table 71: Compounds of formula Ia, wherein each of R² and R⁴ are H, R³ is H, R¹ is OCClF₂ and wherein R⁵ and R⁶ are as defined in one row of table A;
Table 72: Compounds of formula Ia, wherein each of R² and R⁴ are H, R³ is F, R¹ is CH₃ and wherein R⁵ and R⁶ are as defined in one row of table A;
Table 73: Compounds of formula Ia, wherein each of R² and R⁴ are H, R³ is F, R¹ is C₂H₅ and wherein R⁵ and R⁶ are as defined in one row of table A;
Table 74: Compounds of formula Ia, wherein each of R² and R⁴ are H, R³ is F, R¹ is CF₃ and wherein R⁵ and R⁶ are as defined in one row of table A;
Table 75: Compounds of formula Ia, wherein each of R² and R⁴ are H, R³ is F, R¹ is F and wherein R⁵ and R⁶ are as defined in one row of table A;
Table 76: Compounds of formula Ia, wherein each of R² and R⁴ are H, R³ is F, R¹ is Cl and wherein R⁵ and R⁶ are as defined in one row of table A;
Table 77: Compounds of formula Ia, wherein each of R² and R⁴ are H, R³ is F, R¹ is Br and wherein R⁵ and R⁶ are as defined in one row of table A;
Table 78: Compounds of formula Ia, wherein each of R² and R⁴ are H, R³ is F, R¹ is J and wherein R⁵ and R⁶ are as defined in one row of table A;
Table 79: Compounds of formula Ia, wherein each of R² and R⁴ are H, R³ is F, R¹ is OCH₃ and wherein R⁵ and R⁶ are as defined in one row of table A;
Table 80: Compounds of formula Ia, wherein each of R² and R⁴ are H, R³ is F, R¹ is OC₂H₅ and wherein R⁵ and R⁶ are as defined in one row of table A;
Table 81: Compounds of formula Ia, wherein each of R² and R⁴ are H, R³ is F, R¹ is OCF₃ and wherein R⁵ and R⁶ are as defined in one row of table A;
Table 82: Compounds of formula Ia, wherein each of R² and R⁴ are H, R³ is F, R¹ is OCHF₂ and wherein R⁵ and R⁶ are as defined in one row of table A;
Table 83: Compounds of formula Ia, wherein each of R² and R⁴ are H, R³ is F, R¹ is OCClF₂ and wherein R⁵ and R⁶ are as defined in one row of table A;
Table 84: Compounds of formula Ia, wherein each of R² and R⁴ are H, R³ is Cl, R¹ is CH₃ and wherein R⁵ and R⁶ are as defined in one row of table A;
Table 85: Compounds of formula Ia, wherein each of R² and R⁴ are H, R³ is Cl, R¹ is C₂H₅ and wherein R⁵ and R⁶ are as defined in one row of table A;
Table 86: Compounds of formula Ia, wherein each of R² and R⁴ are H, R³ is Cl, R¹ is CF₃ and wherein R⁵ and R⁶ are as defined in one row of table A;
Table 87: Compounds of formula Ia, wherein each of R² and R⁴ are H, R³ is Cl, R¹ is F and wherein R⁵ and R⁶ are as defined in one row of table A;
Table 88: Compounds of formula Ia, wherein each of R² and R⁴ are H, R³ is Cl, R¹ is Cl and wherein R⁵ and R⁶ are as defined in one row of table A;
Table 89: Compounds of formula Ia, wherein each of R² and R⁴ are H, R³ is Cl, R¹ is Br and wherein R⁵ and R⁶ are as defined in one row of table A;
Table 90: Compounds of formula Ia, wherein each of R² and R⁴ are H, R³ is Cl, R¹ is J and wherein R⁵ and R⁶ are as defined in one row of table A;
Table 91: Compounds of formula Ia, wherein each of R² and R⁴ are H, R³ is Cl, R¹ is OCH₃ and wherein R⁵ and R⁶ are as defined in one row of table A;
Table 92: Compounds of formula Ia, wherein each of R² and R⁴ are H, R³ is Cl, R¹ is OC₂H₅ and wherein R⁵ and R⁶ are as defined in one row of table A;
Table 93: Compounds of formula Ia, wherein each of R² and R⁴ are H, R³ is Cl, R¹ is OCF₃ and wherein R⁵ and R⁶ are as defined in one row of table A;
Table 94: Compounds of formula Ia, wherein each of R² and R⁴ are H, R³ is Cl, R¹ is OCHF₂ and wherein R⁵ and R⁶ are as defined in one row of table A;
Table 95: Compounds of formula Ia, wherein each of R² and R⁴ are H, R³ is Cl, R¹ is OCClF₂ and wherein R⁵ and R⁶ are as defined in one row of table A;
Table 96: Compounds of formula Ia, wherein each of R² and R⁴ are H, R³ is Br, R¹ is CH₃ and wherein R⁵ and R⁶ are as defined in one row of table A;
Table 97: Compounds of formula Ia, wherein each of R² and R⁴ are H, R³ is Br, R¹ is C₂H₅ and wherein R⁵ and R⁶ are as defined in one row of table A;
Table 98: Compounds of formula Ia, wherein each of R² and R⁴ are H, R³ is Br, R¹ is CF₃ and wherein R⁵ and R⁶ are as defined in one row of table A;
Table 99: Compounds of formula Ia, wherein each of R² and R⁴ are H, R³ is Br, R¹ is F and wherein R⁵ and R⁶ are as defined in one row of table A;
Table 100: Compounds of formula Ia, wherein each of R² and R⁴ are H, R³ is Br, R¹ is Cl and wherein R⁵ and R⁶ are as defined in one row of table A;
Table 101: Compounds of formula Ia, wherein each of R² and R⁴ are H, R³ is Br, R¹ is Br and wherein R⁵ and R⁶ are as defined in one row of table A;
Table 102: Compounds of formula Ia, wherein each of R² and R⁴ are H, R³ is Br, R¹ is J and wherein R⁵ and R⁶ are as defined in one row of table A;
Table 103: Compounds of formula Ia, wherein each of R² and R⁴ are H, R³ is Br, R¹ is OCH₃ and wherein R⁵ and R⁶ are as defined in one row of table A;
Table 104: Compounds of formula Ia, wherein each of R² and R⁴ are H, R³ is Br, R¹ is OC₂H₅ and wherein R⁵ and R⁶ are as defined in one row of table A;
Table 105: Compounds of formula Ia, wherein each of R² and R⁴ are H, R³ is Br, R¹ is OCF₃ and wherein R⁵ and R⁶ are as defined in one row of table A;
Table 106: Compounds of formula Ia, wherein each of R² and R⁴ are H, R³ is Br, R¹ is OCHF₂ and wherein R⁵ and R⁶ are as defined in one row of table A;
Table 107: Compounds of formula Ia, wherein each of R² and R⁴ are H, R³ is Br, R¹ is OCClF₂ and wherein R⁵ and R⁶ are as defined in one row of table A;
Table 108: Compounds of formula Ia, wherein each of R² and R⁴ are H, R³ is J, R¹ is CH₃ and wherein R⁵ and R⁶ are as defined in one row of table A;
Table 109: Compounds of formula Ia, wherein each of R² and R⁴ are H, R³ is J, R¹ is C₂H₅ and wherein R⁵ and R⁶ are as defined in one row of table A;
Table 110: Compounds of formula Ia, wherein each of R² and R⁴ are H, R³ is J, R¹ is CF₃ and wherein R⁵ and R⁶ are as defined in one row of table A;
Table 111: Compounds of formula Ia, wherein each of R² and R⁴ are H, R³ is J, R¹ is F and wherein R⁵ and R⁶ are as defined in one row of table A;
Table 112: Compounds of formula Ia, wherein each of R² and R⁴ are H, R³ is J, R¹ is Cl and wherein R⁵ and R⁶ are as defined in one row of table A;
Table 113: Compounds of formula Ia, wherein each of R² and R⁴ are H, R³ is J, R¹ is Br and wherein R⁵ and R⁶ are as defined in one row of table A;
Table 114: Compounds of formula Ia, wherein each of R² and R⁴ are H, R³ is J, R¹ is J and wherein R⁵ and R⁶ are as defined in one row of table A;
Table 115: Compounds of formula Ia, wherein each of R² and R⁴ are H, R³ is J, R¹ is OCH₃ and wherein R⁵ and R⁶ are as defined in one row of table A;
Table 116: Compounds of formula Ia, wherein each of R² and R⁴ are H, R³ is J, R¹ is OC₂H₅ and wherein R⁵ and R⁶ are as defined in one row of table A;
Table 117: Compounds of formula Ia, wherein each of R² and R⁴ are H, R³ is J, R¹ is OCF₃ and wherein R⁵ and R⁶ are as defined in one row of table A;
Table 118: Compounds of formula Ia, wherein each of R² and R⁴ are H, R³ is J, R¹ is OCHF₂ and wherein R⁵ and R⁶ are as defined in one row of table A;
Table 119: Compounds of formula Ia, wherein each of R² and R⁴ are H, R³ is J, R¹ is OCClF₂ and wherein R⁵ and R⁶ are as defined in one row of table A;
Table 120: Compounds of formula Ia, wherein each of R³ and R² are H, R⁴ is H, R¹ is C₂H₅ and wherein R⁵ and R⁶ are as defined in one row of table A;
Table 121: Compounds of formula Ia, wherein each of R³ and R² are H, R⁴ is H, R¹ is CF₃ and wherein R⁵ and R⁶ are as defined in one row of table A;
Table 122: Compounds of formula Ia, wherein each of R³ and R² are H, R⁴ is H, R¹ is F and wherein R⁵ and R⁶ are as defined in one row of table A;
Table 123: Compounds of formula Ia, wherein each of R³ and R² are H, R⁴ is H, R¹ is Cl and wherein R⁵ and R⁶ are as defined in one row of table A;
Table 124: Compounds of formula Ia, wherein each of R³ and R² are H, R⁴ is H, R¹ is Br and wherein R⁵ and R⁶ are as defined in one row of table A;
Table 125: Compounds of formula Ia, wherein each of R³ and R² are H, R⁴ is H, R¹ is J and wherein R⁵ and R⁶ are as defined in one row of table A;
Table 126: Compounds of formula Ia, wherein each of R³ and R² are H, R⁴ is H, R¹ is OCH₃ and wherein R⁵ and R⁶ are as defined in one row of table A;
Table 127: Compounds of formula Ia, wherein each of R³ and R² are H, R⁴ is H, R¹ is OC₂H₅ and wherein R⁵ and R⁶ are as defined in one row of table A;
Table 128: Compounds of formula Ia, wherein each of R³ and R² are H, R⁴ is H, R¹ is OCF₃ and wherein R⁵ and R⁶ are as defined in one row of table A;
Table 129: Compounds of formula Ia, wherein each of R³ and R² are H, R⁴ is H, R¹ is OCHF₂ and wherein R⁵ and R⁶ are as defined in one row of table A;
Table 130: Compounds of formula Ia, wherein each of R³ and R² are H, R⁴ is H, R¹ is OCClF₂ and wherein R⁵ and R⁶ are as defined in one row of table A;
Table 131: Compounds of formula Ia, wherein each of R³ and R² are H, R⁴ is F, R¹ is CH₃ and wherein R⁵ and R⁶ are as defined in one row of table A;
Table 132: Compounds of formula Ia, wherein each of R³ and R² are H, R⁴ is F, R¹ is C₂H₅ and wherein R⁵ and R⁶ are as defined in one row of table A;
Table 133: Compounds of formula Ia, wherein each of R³ and R² are H, R⁴ is F, R¹ is CF₃ and wherein R⁵ and R⁶ are as defined in one row of table A;
Table 134: Compounds of formula Ia, wherein each of R³ and R² are H, R⁴ is F, R¹ is F and wherein R⁵ and R⁶ are as defined in one row of table A;
Table 135: Compounds of formula Ia, wherein each of R³ and R² are H, R⁴ is F, R¹ is Cl and wherein R⁵ and R⁶ are as defined in one row of table A;
Table 136: Compounds of formula Ia, wherein each of R³ and R² are H, R⁴ is F, R¹ is Br and wherein R⁵ and R⁶ are as defined in one row of table A;
Table 137: Compounds of formula Ia, wherein each of R³ and R² are H, R⁴ is F, R¹ is J and wherein R⁵ and R⁶ are as defined in one row of table A;
Table 138: Compounds of formula Ia, wherein each of R³ and R² are H, R⁴ is F, R¹ is OCH₃ and wherein R⁵ and R⁶ are as defined in one row of table A;
Table 139: Compounds of formula Ia, wherein each of R³ and R² are H, R⁴ is F, R¹ is OC₂H₅ and wherein R⁵ and R⁶ are as defined in one row of table A;
Table 140: Compounds of formula Ia, wherein each of R³ and R² are H, R⁴ is F, R¹ is OCF₃ and wherein R⁵ and R⁶ are as defined in one row of table A;
Table 141: Compounds of formula Ia, wherein each of R³ and R² are H, R⁴ is F, R¹ is OCHF₂ and wherein R⁵ and R⁶ are as defined in one row of table A;
Table 142: Compounds of formula Ia, wherein each of R³ and R² are H, R⁴ is F, R¹ is OCClF₂ and wherein R⁵ and R⁶ are as defined in one row of table A;
Table 143: Compounds of formula Ia, wherein each of R³ and R² are H, R⁴ is Cl, R¹ is CH₃ and wherein R⁵ and R⁶ are as defined in one row of table A;
Table 144: Compounds of formula Ia, wherein each of R³ and R² are H, R⁴ is Cl, R¹ is C₂H₅ and wherein R⁵ and R⁶ are as defined in one row of table A;
Table 145: Compounds of formula Ia, wherein each of R³ and R² are H, R⁴ is Cl, R¹ is CF₃ and wherein R⁵ and R⁶ are as defined in one row of table A;
Table 146: Compounds of formula Ia, wherein each of R³ and R² are H, R⁴ is Cl, R¹ is F and wherein R⁵ and R⁶ are as defined in one row of table A;
Table 147: Compounds of formula Ia, wherein each of R³ and R² are H, R⁴ is Cl, R¹ is Cl and wherein R⁵ and R⁶ are as defined in one row of table A;
Table 148: Compounds of formula Ia, wherein each of R³ and R² are H, R⁴ is Cl, R¹ is Br and wherein R⁵ and R⁶ are as defined in one row of table A;
Table 149: Compounds of formula Ia, wherein each of R³ and R² are H, R⁴ is Cl, R¹ is J and wherein R⁵ and R⁶ are as defined in one row of table A;
Table 150: Compounds of formula Ia, wherein each of R³ and R² are H, R⁴ is Cl, R¹ is OCH₃ and wherein R⁵ and R⁶ are as defined in one row of table A;
Table 151: Compounds of formula Ia, wherein each of R³ and R² are H, R⁴ is Cl, R¹ is OC₂H₅ and wherein R⁵ and R⁶ are as defined in one row of table A;
Table 152: Compounds of formula Ia, wherein each of R³ and R² are H, R⁴ is Cl, R¹ is OCF₃ and wherein R⁵ and R⁶ are as defined in one row of table A;
Table 153: Compounds of formula Ia, wherein each of R³ and R² are H, R⁴ is Cl, R¹ is OCHF₂ and wherein R⁵ and R⁶ are as defined in one row of table A;
Table 154: Compounds of formula Ia, wherein each of R³ and R² are H, R⁴ is Cl, R¹ is OCClF₂ and wherein R⁵ and R⁶ are as defined in one row of table A;
Table 155: Compounds of formula Ia, wherein each of R³ and R² are H, R⁴ is Br, R¹ is CH₃ and wherein R⁵ and R⁶ are as defined in one row of table A;
Table 156: Compounds of formula Ia, wherein each of R³ and R² are H, R⁴ is Br, R¹ is C₂H₅ and wherein R⁵ and R⁶ are as defined in one row of table A;
Table 157: Compounds of formula Ia, wherein each of R³ and R² are H, R⁴ is Br, R¹ is CF₃ and wherein R⁵ and R⁶ are as defined in one row of table A;
Table 158: Compounds of formula Ia, wherein each of R³ and R² are H, R⁴ is Br, R¹ is F and wherein R⁵ and R⁶ are as defined in one row of table A;
Table 159: Compounds of formula Ia, wherein each of R³ and R² are H, R⁴ is Br, R¹ is Cl and wherein R⁵ and R⁶ are as defined in one row of table A;
Table 160: Compounds of formula Ia, wherein each of R³ and R² are H, R⁴ is Br, R¹ is Br and wherein R⁵ and R⁶ are as defined in one row of table A;
Table 161: Compounds of formula Ia, wherein each of R³ and R² are H, R⁴ is Br, R¹ is J and wherein R⁵ and R⁶ are as defined in one row of table A;
Table 162: Compounds of formula Ia, wherein each of R³ and R² are H, R⁴ is Br, R¹ is OCH₃ and wherein R⁵ and R⁶ are as defined in one row of table A;
Table 163: Compounds of formula Ia, wherein each of R³ and R² are H, R⁴ is Br, R¹ is OC₂H₅ and wherein R⁵ and R⁶ are as defined in one row of table A;
Table 164: Compounds of formula Ia, wherein each of R³ and R² are H, R⁴ is Br, R¹ is OCF₃ and wherein R⁵ and R⁶ are as defined in one row of table A;
Table 165: Compounds of formula Ia, wherein each of R³ and R² are H, R⁴ is Br, R¹ is OCHF₂ and wherein R⁵ and R⁶ are as defined in one row of table A;
Table 166: Compounds of formula Ia, wherein each of R³ and R² are H, R⁴ is Br, R¹ is OCClF₂ and wherein R⁵ and R⁶ are as defined in one row of table A;
Table 167: Compounds of formula Ia, wherein each of R³ and R² are H, R⁴ is J, R¹ is CH₃ and wherein R⁵ and R⁶ are as defined in one row of table A;
Table 168: Compounds of formula Ia, wherein each of R³ and R² are H, R⁴ is J, R¹ is C₂H₅ and wherein R⁵ and R⁶ are as defined in one row of table A;
Table 169: Compounds of formula Ia, wherein each of R³ and R² are H, R⁴ is J, R¹ is CF₃ and wherein R⁵ and R⁶ are as defined in one row of table A;
Table 170: Compounds of formula Ia, wherein each of R³ and R² are H, R⁴ is J, R¹ is F and wherein R⁵ and R⁶ are as defined in one row of table A;
Table 171: Compounds of formula Ia, wherein each of R³ and R² are H, R⁴ is J, R¹ is Cl and wherein R⁵ and R⁶ are as defined in one row of table A;
Table 172: Compounds of formula Ia, wherein each of R³ and R² are H, R⁴ is J, R¹ is Br and wherein R⁵ and R⁶ are as defined in one row of table A;
Table 173: Compounds of formula Ia, wherein each of R³ and R² are H, R⁴ is J, R¹ is J and wherein R⁵ and R⁶ are as defined in one row of table A;
Table 174: Compounds of formula Ia, wherein each of R³ and R² are H, R⁴ is J, R¹ is OCH₃ and wherein R⁵ and R⁶ are as defined in one row of table A;
Table 175: Compounds of formula Ia, wherein each of R³ and R² are H, R⁴ is J, R¹ is OC₂H₅ and wherein R⁵ and R⁶ are as defined in one row of table A;
Table 176: Compounds of formula Ia, wherein each of R³ and R² are H, R⁴ is J, R¹ is OCF₃ and wherein R⁵ and R⁶ are as defined in one row of table A;
Table 177: Compounds of formula Ia, wherein each of R³ and R² are H, R⁴ is J, R¹ is OCHF₂ and wherein R⁵ and R⁶ are as defined in one row of table A;
Table 178: Compounds of formula Ia, wherein each of R³ and R² are H, R⁴ is J, R¹ is OCClF₂ and wherein R⁵ and R⁶ are as defined in one row of table A;
Table 179: Compounds of formula Ib, wherein each of R³ and R⁴ are H, R² is H, R¹ is CH₃ and wherein R⁵ and R⁶ are as defined in one row of table A;
Table 180: Compounds of formula Ib, wherein each of R³ and R⁴ are H, R² is H, R¹ is C₂H₅ and wherein R⁵ and R⁶ are as defined in one row of table A;
Table 181: Compounds of formula Ib, wherein each of R³ and R⁴ are H, R² is H, R¹ is CF₃ and wherein R⁵ and R⁶ are as defined in one row of table A;
Table 182: Compounds of formula Ib, wherein each of R³ and R⁴ are H, R² is H, R¹ is F and wherein R⁵ and R⁶ are as defined in one row of table A;
Table 183: Compounds of formula Ib, wherein each of R³ and R⁴ are H, R² is H, R¹ is Cl and wherein R⁵ and R⁶ are as defined in one row of table A;
Table 184: Compounds of formula Ib, wherein each of R³ and R⁴ are H, R² is H, R¹ is Br and wherein R⁵ and R⁶ are as defined in one row of table A;
Table 185: Compounds of formula Ib, wherein each of R³ and R⁴ are H, R² is H, R¹ is J and wherein R⁵ and R⁶ are as defined in one row of table A;
Table 186: Compounds of formula Ib, wherein each of R³ and R⁴ are H, R² is H, R¹ is OCH₃ and wherein R⁵ and R⁶ are as defined in one row of table A;
Table 187: Compounds of formula Ib, wherein each of R³ and R⁴ are H, R² is H, R¹ is OC₂H₅ and wherein R⁵ and R⁶ are as defined in one row of table A;
Table 188: Compounds of formula Ib, wherein each of R³ and R⁴ are H, R² is H, R¹ is OCF₃ and wherein R⁵ and R⁶ are as defined in one row of table A;
Table 189: Compounds of formula Ib, wherein each of R³ and R⁴ are H, R² is H, R¹ is OCHF₂ and wherein R⁵ and R⁶ are as defined in one row of table A;
Table 190: Compounds of formula Ib, wherein each of R³ and R⁴ are H, R² is H, R¹ is OCClF₂ and wherein R⁵ and R⁶ are as defined in one row of table A;
Table 191: Compounds of formula Ib, wherein each of R³ and R⁴ are H, R² is F, R¹ is CH₃ and wherein R⁵ and R⁶ are as defined in one row of table A;
Table 192: Compounds of formula Ib, wherein each of R³ and R⁴ are H, R² is F, R¹ is C₂H₅ and wherein R⁵ and R⁶ are as defined in one row of table A;
Table 193: Compounds of formula Ib, wherein each of R³ and R⁴ are H, R² is F, R¹ is CF₃ and wherein R⁵ and R⁶ are as defined in one row of table A;
Table 194: Compounds of formula Ib, wherein each of R³ and R⁴ are H, R² is F, R¹ is F and wherein R⁵ and R⁶ are as defined in one row of table A;
Table 195: Compounds of formula Ib, wherein each of R³ and R⁴ are H, R² is F, R¹ is Cl and wherein R⁵ and R⁶ are as defined in one row of table A;
Table 196: Compounds of formula Ib, wherein each of R³ and R⁴ are H, R² is F, R¹ is Br and wherein R⁵ and R⁶ are as defined in one row of table A;
Table 197: Compounds of formula Ib, wherein each of R³ and R⁴ are H, R² is F, R¹ is J and wherein R⁵ and R⁶ are as defined in one row of table A;
Table 198: Compounds of formula Ib, wherein each of R³ and R⁴ are H, R² is F, R¹ is OCH₃ and wherein R⁵ and R⁶ are as defined in one row of table A;
Table 199: Compounds of formula Ib, wherein each of R³ and R⁴ are H, R² is F, R¹ is OC₂H₅ and wherein R⁵ and R⁶ are as defined in one row of table A;
Table 200: Compounds of formula Ib, wherein each of R³ and R⁴ are H, R² is F, R¹ is OCF₃ and wherein R⁵ and R⁶ are as defined in one row of table A;
Table 201: Compounds of formula Ib, wherein each of R³ and R⁴ are H, R² is F, R¹ is OCHF₂ and wherein R⁵ and R⁶ are as defined in one row of table A;
Table 202: Compounds of formula Ib, wherein each of R³ and R⁴ are H, R² is F, R¹ is OCClF₂ and wherein R⁵ and R⁶ are as defined in one row of table A;
Table 203: Compounds of formula Ib, wherein each of R³ and R⁴ are H, R² is Cl, R¹ is CH₃ and wherein R⁵ and R⁶ are as defined in one row of table A;
Table 204: Compounds of formula Ib, wherein each of R³ and R⁴ are H, R² is Cl, R¹ is C₂H₅ and wherein R⁵ and R⁶ are as defined in one row of table A;
Table 205: Compounds of formula Ib, wherein each of R³ and R⁴ are H, R² is Cl, R¹ is CF₃ and wherein R⁵ and R⁶ are as defined in one row of table A;
Table 206: Compounds of formula Ib, wherein each of R³ and R⁴ are H, R² is Cl, R¹ is F and wherein R⁵ and R⁶ are as defined in one row of table A;
Table 207: Compounds of formula Ib, wherein each of R³ and R⁴ are H, R² is Cl, R¹ is Cl and wherein R⁵ and R⁶ are as defined in one row of table A;
Table 208: Compounds of formula Ib, wherein each of R³ and R⁴ are H, R² is Cl, R¹ is Br and wherein R⁵ and R⁶ are as defined in one row of table A;
Table 209: Compounds of formula Ib, wherein each of R³ and R⁴ are H, R² is Cl, R¹ is J and wherein R⁵ and R⁶ are as defined in one row of table A;
Table 210: Compounds of formula Ib, wherein each of R³ and R⁴ are H, R² is Cl, R¹ is OCH₃ and wherein R⁵ and R⁶ are as defined in one row of table A;
Table 211: Compounds of formula Ib, wherein each of R³ and R⁴ are H, R² is Cl, R¹ is OC₂H₅ and wherein R⁵ and R⁶ are as defined in one row of table A;
Table 212: Compounds of formula Ib, wherein each of R³ and R⁴ are H, R² is Cl, R¹ is OCF₃ and wherein R⁵ and R⁶ are as defined in one row of table A;
Table 213: Compounds of formula Ib, wherein each of R³ and R⁴ are H, R² is Cl, R¹ is OCHF₂ and wherein R⁵ and R⁶ are as defined in one row of table A;
Table 214: Compounds of formula Ib, wherein each of R³ and R⁴ are H, R² is Cl, R¹ is OCClF₂ and wherein R⁵ and R⁶ are as defined in one row of table A;
Table 215: Compounds of formula Ib, wherein each of R³ and R⁴ are H, R² is Br, R¹ is CH₃ and wherein R⁵ and R⁶ are as defined in one row of table A;
Table 216: Compounds of formula Ib, wherein each of R³ and R⁴ are H, R² is Br, R¹ is C₂H₅ and wherein R⁵ and R⁶ are as defined in one row of table A;
Table 217: Compounds of formula Ib, wherein each of R³ and R⁴ are H, R² is Br, R¹ is CF₃ and wherein R⁵ and R⁶ are as defined in one row of table A;
Table 218: Compounds of formula Ib, wherein each of R³ and R⁴ are H, R² is Br, R¹ is F and wherein R⁵ and R⁶ are as defined in one row of table A;
Table 219: Compounds of formula Ib, wherein each of R³ and R⁴ are H, R² is Br, R¹ is Cl and wherein R⁵ and R⁶ are as defined in one row of table A;
Table 220: Compounds of formula Ib, wherein each of R³ and R⁴ are H, R² is Br, R¹ is Br and wherein R⁵ and R⁶ are as defined in one row of table A;
Table 221: Compounds of formula Ib, wherein each of R³ and R⁴ are H, R² is Br, R¹ is J and wherein R⁵ and R⁶ are as defined in one row of table A;
Table 222: Compounds of formula Ib, wherein each of R³ and R⁴ are H, R² is Br, R¹ is OCH₃ and wherein R⁵ and R⁶ are as defined in one row of table A;
Table 223: Compounds of formula Ib, wherein each of R³ and R⁴ are H, R² is Br, R¹ is OC₂H₅ and wherein R⁵ and R⁶ are as defined in one row of table A;
Table 224: Compounds of formula Ib, wherein each of R³ and R⁴ are H, R² is Br, R¹ is OCF₃ and wherein R⁵ and R⁶ are as defined in one row of table A;
Table 225: Compounds of formula Ib, wherein each of R³ and R⁴ are H, R² is Br, R¹ is OCHF₂ and wherein R⁵ and R⁶ are as defined in one row of table A;
Table 226: Compounds of formula Ib, wherein each of R³ and R⁴ are H, R² is Br, R¹ is OCClF₂ and wherein R⁵ and R⁶ are as defined in one row of table A;
Table 227: Compounds of formula Ib, wherein each of R³ and R⁴ are H, R² is J, R¹ is CH₃ and wherein R⁵ and R⁶ are as defined in one row of table A;
Table 228: Compounds of formula Ib, wherein each of R³ and R⁴ are H, R² is J, R¹ is C₂H₅ and wherein R⁵ and R⁶ are as defined in one row of table A;
Table 229: Compounds of formula Ib, wherein each of R³ and R⁴ are H, R² is J, R¹ is CF₃ and wherein R⁵ and R⁶ are as defined in one row of table A;
Table 230: Compounds of formula Ib, wherein each of R³ and R⁴ are H, R² is J, R¹ is F and wherein R⁵ and R⁶ are as defined in one row of table A;
Table 231: Compounds of formula Ib, wherein each of R³ and R⁴ are H, R² is J, R¹ is Cl and wherein R⁵ and R⁶ are as defined in one row of table A;
Table 232: Compounds of formula Ib, wherein each of R³ and R⁴ are H, R² is J, R¹ is Br and wherein R⁵ and R⁶ are as defined in one row of table A;
Table 233: Compounds of formula Ib, wherein each of R³ and R⁴ are H, R² is J, R¹ is J and wherein R⁵ and R⁶ are as defined in one row of table A;
Table 234: Compounds of formula Ib, wherein each of R³ and R⁴ are H, R² is J, R¹ is OCH₃ and wherein R⁵ and R⁶ are as defined in one row of table A;
Table 235: Compounds of formula Ib, wherein each of R³ and R⁴ are H, R² is J, R¹ is OC₂H₅ and wherein R⁵ and R⁶ are as defined in one row of table A;
Table 236: Compounds of formula Ib, wherein each of R³ and R⁴ are H, R² is J, R¹ is OCF₃ and wherein R⁵ and R⁶ are as defined in one row of table A;
Table 237: Compounds of formula Ib, wherein each of R³ and R⁴ are H, R² is J, R¹ is OCHF₂ and wherein R⁵ and R⁶ are as defined in one row of table A;
Table 238: Compounds of formula Ib, wherein each of R³ and R⁴ are H, R² is J, R¹ is OCClF₂ and wherein R⁵ and R⁶ are as defined in one row of table A;
Table 239: Compounds of formula Ib, wherein each of R² and R⁴ are H, R³ is H, R¹ is C₂H₅ and wherein R⁵ and R⁶ are as defined in one row of table A;
Table 240: Compounds of formula Ib, wherein each of R² and R⁴ are H, R³ is H, R¹ is CF₃ and wherein R⁵ and R⁶ are as defined in one row of table A;
Table 241: Compounds of formula Ib, wherein each of R² and R⁴ are H, R³ is H, R¹ is F and wherein R⁵ and R⁶ are as defined in one row of table A;
Table 242: Compounds of formula Ib, wherein each of R² and R⁴ are H, R³ is H, R¹ is Cl and wherein R⁵ and R⁶ are as defined in one row of table A;
Table 243: Compounds of formula Ib, wherein each of R² and R⁴ are H, R³ is H, R¹ is Br and wherein R⁵ and R⁶ are as defined in one row of table A;
Table 244: Compounds of formula Ib, wherein each of R² and R⁴ are H, R³ is H, R¹ is J and wherein R⁵ and R⁶ are as defined in one row of table A;
Table 245: Compounds of formula Ib, wherein each of R² and R⁴ are H, R³ is H, R¹ is OCH₃ and wherein R⁵ and R⁶ are as defined in one row of table A;
Table 246: Compounds of formula Ib, wherein each of R² and R⁴ are H, R³ is H, R¹ is OC₂H₅ and wherein R⁵ and R⁶ are as defined in one row of table A;
Table 247: Compounds of formula Ib, wherein each of R² and R⁴ are H, R³ is H, R¹ is OCF₃ and wherein R⁵ and R⁶ are as defined in one row of table A;
Table 248: Compounds of formula Ib, wherein each of R² and R⁴ are H, R³ is H, R¹ is OCHF₂ and wherein R⁵ and R⁶ are as defined in one row of table A;
Table 249: Compounds of formula Ib, wherein each of R² and R⁴ are H, R³ is H, R¹ is OCClF₂ and wherein R⁵ and R⁶ are as defined in one row of table A;
Table 250: Compounds of formula Ib, wherein each of R² and R⁴ are H, R³ is F, R¹ is CH₃ and wherein R⁵ and R⁶ are as defined in one row of table A;
Table 251: Compounds of formula Ib, wherein each of R² and R⁴ are H, R³ is F, R¹ is C₂H₅ and wherein R⁵ and R⁶ are as defined in one row of table A;
Table 252: Compounds of formula Ib, wherein each of R² and R⁴ are H, R³ is F, R¹ is CF₃ and wherein R⁵ and R⁶ are as defined in one row of table A;
Table 253: Compounds of formula Ib, wherein each of R² and R⁴ are H, R³ is F, R¹ is F and wherein R⁵ and R⁶ are as defined in one row of table A;
Table 254: Compounds of formula Ib, wherein each of R² and R⁴ are H, R³ is F, R¹ is Cl and wherein R⁵ and R⁶ are as defined in one row of table A;
Table 255: Compounds of formula Ib, wherein each of R² and R⁴ are H, R³ is F, R¹ is Br and wherein R⁵ and R⁶ are as defined in one row of table A;
Table 256: Compounds of formula Ib, wherein each of R² and R⁴ are H, R³ is F, R¹ is J and wherein R⁵ and R⁶ are as defined in one row of table A;
Table 257: Compounds of formula Ib, wherein each of R² and R⁴ are H, R³ is F, R¹ is OCH₃ and wherein R⁵ and R⁶ are as defined in one row of table A;
Table 258: Compounds of formula Ib, wherein each of R² and R⁴ are H, R³ is F, R¹ is OC₂H₅ and wherein R⁵ and R⁶ are as defined in one row of table A;
Table 259: Compounds of formula Ib, wherein each of R² and R⁴ are H, R³ is F, R¹ is OCF₃ and wherein R⁵ and R⁶ are as defined in one row of table A;
Table 260: Compounds of formula Ib, wherein each of R² and R⁴ are H, R³ is F, R¹ is OCHF₂ and wherein R⁵ and R⁶ are as defined in one row of table A;
Table 261: Compounds of formula Ib, wherein each of R² and R⁴ are H, R³ is F, R¹ is OCClF₂ and wherein R⁵ and R⁶ are as defined in one row of table A;
Table 262: Compounds of formula Ib, wherein each of R² and R⁴ are H, R³ is Cl, R¹ is CH₃ and wherein R⁵ and R⁶ are as defined in one row of table A;
Table 263: Compounds of formula Ib, wherein each of R² and R⁴ are H, R³ is Cl, R¹ is C₂H₅ and wherein R⁵ and R⁶ are as defined in one row of table A;
Table 264: Compounds of formula Ib, wherein each of R² and R⁴ are H, R³ is Cl, R¹ is CF₃ and wherein R⁵ and R⁶ are as defined in one row of table A;
Table 265: Compounds of formula Ib, wherein each of R² and R⁴ are H, R³ is Cl, R¹ is F and wherein R⁵ and R⁶ are as defined in one row of table A;
Table 266: Compounds of formula Ib, wherein each of R² and R⁴ are H, R³ is Cl, R¹ is Cl and wherein R⁵ and R⁶ are as defined in one row of table A;
Table 267: Compounds of formula Ib, wherein each of R² and R⁴ are H, R³ is Cl, R¹ is Br and wherein R⁵ and R⁶ are as defined in one row of table A;
Table 268: Compounds of formula Ib, wherein each of R² and R⁴ are H, R³ is Cl, R¹ is J and wherein R⁵ and R⁶ are as defined in one row of table A;
Table 269: Compounds of formula Ib, wherein each of R² and R⁴ are H, R³ is Cl, R¹ is OCH₃ and wherein R⁵ and R⁶ are as defined in one row of table A;
Table 270: Compounds of formula Ib, wherein each of R² and R⁴ are H, R³ is Cl, R¹ is OC₂H₅ and wherein R⁵ and R⁶ are as defined in one row of table A;
Table 271: Compounds of formula Ib, wherein each of R² and R⁴ are H, R³ is Cl, R¹ is OCF₃ and wherein R⁵ and R⁶ are as defined in one row of table A;
Table 272: Compounds of formula Ib, wherein each of R² and R⁴ are H, R³ is Cl, R¹ is OCHF₂ and wherein R⁵ and R⁶ are as defined in one row of table A;
Table 273: Compounds of formula Ib, wherein each of R² and R⁴ are H, R³ is Cl, R¹ is OCClF₂ and wherein R⁵ and R⁶ are as defined in one row of table A;
Table 274: Compounds of formula Ib, wherein each of R² and R⁴ are H, R³ is Br, R¹ is CH₃ and wherein R⁵ and R⁶ are as defined in one row of table A;
Table 275: Compounds of formula Ib, wherein each of R² and R⁴ are H, R³ is Br, R¹ is C₂H₅ and wherein R⁵ and R⁶ are as defined in one row of table A;
Table 276: Compounds of formula Ib, wherein each of R² and R⁴ are H, R³ is Br, R¹ is CF₃ and wherein R⁵ and R⁶ are as defined in one row of table A;
Table 277: Compounds of formula Ib, wherein each of R² and R⁴ are H, R³ is Br, R¹ is F and wherein R⁵ and R⁶ are as defined in one row of table A;
Table 278: Compounds of formula Ib, wherein each of R² and R⁴ are H, R³ is Br, R¹ is Cl and wherein R⁵ and R⁶ are as defined in one row of table A;
Table 279: Compounds of formula Ib, wherein each of R² and R⁴ are H, R³ is Br, R¹ is Br and wherein R⁵ and R⁶ are as defined in one row of table A;
Table 280: Compounds of formula Ib, wherein each of R² and R⁴ are H, R³ is Br, R¹ is J and wherein R⁵ and R⁶ are as defined in one row of table A;
Table 281: Compounds of formula Ib, wherein each of R² and R⁴ are H, R³ is Br, R¹ is OCH₃ and wherein R⁵ and R⁶ are as defined in one row of table A;
Table 282: Compounds of formula Ib, wherein each of R² and R⁴ are H, R³ is Br, R¹ is OC₂H₅ and wherein R⁵ and R⁶ are as defined in one row of table A;
Table 283: Compounds of formula Ib, wherein each of R² and R⁴ are H, R³ is Br, R¹ is OCF₃ and wherein R⁵ and R⁶ are as defined in one row of table A;
Table 284: Compounds of formula Ib, wherein each of R² and R⁴ are H, R³ is Br, R¹ is OCHF₂ and wherein R⁵ and R⁶ are as defined in one row of table A;
Table 285: Compounds of formula Ib, wherein each of R² and R⁴ are H, R³ is Br, R¹ is OCClF₂ and wherein R⁵ and R⁶ are as defined in one row of table A;
Table 286: Compounds of formula Ib, wherein each of R² and R⁴ are H, R³ is J, R¹ is CH₃ and wherein R⁵ and R⁶ are as defined in one row of table A;
Table 287: Compounds of formula Ib, wherein each of R² and R⁴ are H, R³ is J, R¹ is C₂H₅ and wherein R⁵ and R⁶ are as defined in one row of table A;
Table 288: Compounds of formula Ib, wherein each of R² and R⁴ are H, R³ is J, R¹ is CF₃ and wherein R⁵ and R⁶ are as defined in one row of table A;
Table 289: Compounds of formula Ib, wherein each of R² and R⁴ are H, R³ is J, R¹ is F and wherein R⁵ and R⁶ are as defined in one row of table A;
Table 290: Compounds of formula Ib, wherein each of R² and R⁴ are H, R³ is J, R¹ is Cl and wherein R⁵ and R⁶ are as defined in one row of table A;
Table 291: Compounds of formula Ib, wherein each of R² and R⁴ are H, R³ is J, R¹ is Br and wherein R⁵ and R⁶ are as defined in one row of table A;
Table 292: Compounds of formula Ib, wherein each of R² and R⁴ are H, R³ is J, R¹ is J and wherein R⁵ and R⁶ are as defined in one row of table A;
Table 293: Compounds of formula Ib, wherein each of R² and R⁴ are H, R³ is J, R¹ is OCH₃ and wherein R⁵ and R⁶ are as defined in one row of table A;
Table 294: Compounds of formula Ib, wherein each of R² and R⁴ are H, R³ is J, R¹ is OC₂H₅ and wherein R⁵ and R⁶ are as defined in one row of table A;
Table 295: Compounds of formula Ib, wherein each of R² and R⁴ are H, R³ is J, R¹ is OCF₃ and wherein R⁵ and R⁶ are as defined in one row of table A;
Table 296: Compounds of formula Ib, wherein each of R² and R⁴ are H, R³ is J, R¹ is OCHF₂ and wherein R⁵ and R⁶ are as defined in one row of table A;
Table 297: Compounds of formula Ib, wherein each of R² and R⁴ are H, R³ is J, R¹ is OCClF₂ and wherein R⁵ and R⁶ are as defined in one row of table A;
Table 298: Compounds of formula Ib, wherein each of R³ and R² are H, R⁴ is H, R¹ is C₂H₅ and wherein R⁵ and R⁶ are as defined in one row of table A;
Table 299: Compounds of formula Ib, wherein each of R³ and R² are H, R⁴ is H, R¹ is CF₃ and wherein R⁵ and R⁶ are as defined in one row of table A;
Table 300: Compounds of formula Ib, wherein each of R³ and R² are H, R⁴ is H, R¹ is F and wherein R⁵ and R⁶ are as defined in one row of table A;
Table 301: Compounds of formula Ib, wherein each of R³ and R² are H, R⁴ is H, R¹ is Cl and wherein R⁵ and R⁶ are as defined in one row of table A;
Table 302: Compounds of formula Ib, wherein each of R³ and R² are H, R⁴ is H, R¹ is Br and wherein R⁵ and R⁶ are as defined in one row of table A;
Table 303: Compounds of formula Ib, wherein each of R³ and R² are H, R⁴ is H, R¹ is J and wherein R⁵ and R⁶ are as defined in one row of table A;
Table 304: Compounds of formula Ib, wherein each of R³ and R² are H, R⁴ is H, R¹ is OCH₃ and wherein R⁵ and R⁶ are as defined in one row of table A;
Table 305: Compounds of formula Ib, wherein each of R³ and R² are H, R⁴ is H, R¹ is OC₂H₅ and wherein R⁵ and R⁶ are as defined in one row of table A;
Table 306: Compounds of formula Ib, wherein each of R³ and R² are H, R⁴ is H, R¹ is OCF₃ and wherein R⁵ and R⁶ are as defined in one row of table A;
Table 307: Compounds of formula Ib, wherein each of R³ and R² are H, R⁴ is H, R¹ is OCHF₂ and wherein R⁵ and R⁶ are as defined in one row of table A;
Table 308: Compounds of formula Ib, wherein each of R³ and R² are H, R⁴ is H, R¹ is OCClF₂ and wherein R⁵ and R⁶ are as defined in one row of table A;
Table 309: Compounds of formula Ib, wherein each of R³ and R² are H, R⁴ is F, R¹ is CH₃ and wherein R⁵ and R⁶ are as defined in one row of table A;
Table 310: Compounds of formula Ib, wherein each of R³ and R² are H, R⁴ is F, R¹ is C₂H₅ and wherein R⁵ and R⁶ are as defined in one row of table A;
Table 311: Compounds of formula Ib, wherein each of R³ and R² are H, R⁴ is F, R¹ is CF₃ and wherein R⁵ and R⁶ are as defined in one row of table A;
Table 312: Compounds of formula Ib, wherein each of R³ and R² are H, R⁴ is F, R¹ is F and wherein R⁵ and R⁶ are as defined in one row of table A;
Table 313: Compounds of formula Ib, wherein each of R³ and R² are H, R⁴ is F, R¹ is Cl and wherein R⁵ and R⁶ are as defined in one row of table A;
Table 314: Compounds of formula Ib, wherein each of R³ and R² are H, R⁴ is F, R¹ is Br and wherein R⁵ and R⁶ are as defined in one row of table A;
Table 315: Compounds of formula Ib, wherein each of R³ and R² are H, R⁴ is F, R¹ is J and wherein R⁵ and R⁶ are as defined in one row of table A;
Table 316: Compounds of formula Ib, wherein each of R³ and R² are H, R⁴ is F, R¹ is OCH₃ and wherein R⁵ and R⁶ are as defined in one row of table A;
Table 317: Compounds of formula Ib, wherein each of R³ and R² are H, R⁴ is F, R¹ is OC₂H₅ and wherein R⁵ and R⁶ are as defined in one row of table A;
Table 318: Compounds of formula Ib, wherein each of R³ and R² are H, R⁴ is F, R¹ is OCF₃ and wherein R⁵ and R⁶ are as defined in one row of table A;
Table 319: Compounds of formula Ib, wherein each of R³ and R² are H, R⁴ is F, R¹ is OCHF₂ and wherein R⁵ and R⁶ are as defined in one row of table A;
Table 320: Compounds of formula Ib, wherein each of R³ and R² are H, R⁴ is F, R¹ is OCClF₂ and wherein R⁵ and R⁶ are as defined in one row of table A;
Table 321: Compounds of formula Ib, wherein each of R³ and R² are H, R⁴ is Cl, R¹ is CH₃ and wherein R⁵ and R⁶ are as defined in one row of table A;
Table 322: Compounds of formula Ib, wherein each of R³ and R² are H, R⁴ is Cl, R¹ is C₂H₅ and wherein R⁵ and R⁶ are as defined in one row of table A;
Table 323: Compounds of formula Ib, wherein each of R³ and R² are H, R⁴ is Cl, R¹ is CF₃ and wherein R⁵ and R⁶ are as defined in one row of table A;
Table 324: Compounds of formula Ib, wherein each of R³ and R² are H, R⁴ is Cl, R¹ is F and wherein R⁵ and R⁶ are as defined in one row of table A;
Table 325: Compounds of formula Ib, wherein each of R³ and R² are H, R⁴ is Cl, R¹ is Cl and wherein R⁵ and R⁶ are as defined in one row of table A;
Table 326: Compounds of formula Ib, wherein each of R³ and R² are H, R⁴ is Cl, R¹ is Br and wherein R⁵ and R⁶ are as defined in one row of table A;
Table 327: Compounds of formula Ib, wherein each of R³ and R² are H, R⁴ is Cl, R¹ is J and wherein R⁵ and R⁶ are as defined in one row of table A;
Table 328: Compounds of formula Ib, wherein each of R³ and R² are H, R⁴ is Cl, R¹ is OCH₃ and wherein R⁵ and R⁶ are as defined in one row of table A;
Table 329: Compounds of formula Ib, wherein each of R³ and R² are H, R⁴ is Cl, R¹ is OC₂H₅ and wherein R⁵ and R⁶ are as defined in one row of table A;
Table 330: Compounds of formula Ib, wherein each of R³ and R² are H, R⁴ is Cl, R¹ is OCF₃ and wherein R⁵ and R⁶ are as defined in one row of table A;
Table 331: Compounds of formula Ib, wherein each of R³ and R² are H, R⁴ is Cl, R¹ is OCHF₂ and wherein R⁵ and R⁶ are as defined in one row of table A;
Table 332: Compounds of formula Ib, wherein each of R³ and R² are H, R⁴ is Cl, R¹ is OCClF₂ and wherein R⁵ and R⁶ are as defined in one row of table A;
Table 333: Compounds of formula Ib, wherein each of R³ and R² are H, R⁴ is Br, R¹ is CH₃ and wherein R⁵ and R⁶ are as defined in one row of table A;
Table 334: Compounds of formula Ib, wherein each of R³ and R² are H, R⁴ is Br, R¹ is C₂H₅ and wherein R⁵ and R⁶ are as defined in one row of table A;
Table 335: Compounds of formula Ib, wherein each of R³ and R² are H, R⁴ is Br, R¹ is CF₃ and wherein R⁵ and R⁶ are as defined in one row of table A;
Table 336: Compounds of formula Ib, wherein each of R³ and R² are H, R⁴ is Br, R¹ is F and wherein R⁵ and R⁶ are as defined in one row of table A;
Table 337: Compounds of formula Ib, wherein each of R³ and R² are H, R⁴ is Br, R¹ is Cl and wherein R⁵ and R⁶ are as defined in one row of table A;
Table 338: Compounds of formula Ib, wherein each of R³ and R² are H, R⁴ is Br, R¹ is Br and wherein R⁵ and R⁶ are as defined in one row of table A;
Table 339: Compounds of formula Ib, wherein each of R³ and R² are H, R⁴ is Br, R¹ is J and wherein R⁵ and R⁶ are as defined in one row of table A;
Table 340: Compounds of formula Ib, wherein each of R³ and R² are H, R⁴ is Br, R¹ is OCH₃ and wherein R⁵ and R⁶ are as defined in one row of table A;
Table 341: Compounds of formula Ib, wherein each of R³ and R² are H, R⁴ is Br, R¹ is OC₂H₅ and wherein R⁵ and R⁶ are as defined in one row of table A;
Table 342: Compounds of formula Ib, wherein each of R³ and R² are H, R⁴ is Br, R¹ is OCF₃ and wherein R⁵ and R⁶ are as defined in one row of table A;
Table 343: Compounds of formula Ib, wherein each of R³ and R² are H, R⁴ is Br, R¹ is OCHF₂ and wherein R⁵ and R⁶ are as defined in one row of table A;
Table 344: Compounds of formula Ib, wherein each of R³ and R² are H, R⁴ is Br, R¹ is OCClF₂ and wherein R⁵ and R⁶ are as defined in one row of table A;
Table 345: Compounds of formula Ib, wherein each of R³ and R² are H, R⁴ is J, R¹ is CH₃ and wherein R⁵ and R⁶ are as defined in one row of table A;
Table 346: Compounds of formula Ib, wherein each of R³ and R² are H, R⁴ is J, R¹ is C₂H₅ and wherein R⁵ and R⁶ are as defined in one row of table A;
Table 347: Compounds of formula Ib, wherein each of R³ and R² are H, R⁴ is J, R¹ is CF₃ and wherein R⁵ and R⁶ are as defined in one row of table A;
Table 348: Compounds of formula Ib, wherein each of R³ and R² are H, R⁴ is J, R¹ is F and wherein R⁵ and R⁶ are as defined in one row of table A;
Table 349: Compounds of formula Ib, wherein each of R³ and R² are H, R⁴ is J, R¹ is Cl and wherein R⁵ and R⁶ are as defined in one row of table A;
Table 350: Compounds of formula Ib, wherein each of R³ and R² are H, R⁴ is J, R¹ is Br and wherein R⁵ and R⁶ are as defined in one row of table A;
Table 351: Compounds of formula Ib, wherein each of R³ and R² are H, R⁴ is J, R¹ is J and wherein R⁵ and R⁶ are as defined in one row of table A;
Table 352: Compounds of formula Ib, wherein each of R³ and R² are H, R⁴ is J, R¹ is OCH₃ and wherein R⁵ and R⁶ are as defined in one row of table A;
Table 353: Compounds of formula Ib, wherein each of R³ and R² are H, R⁴ is J, R¹ is OC₂H₅ and wherein R⁵ and R⁶ are as defined in one row of table A;
Table 354: Compounds of formula Ib, wherein each of R³ and R² are H, R⁴ is J, R¹ is OCF₃ and wherein R⁵ and R⁶ are as defined in one row of table A;
Table 355: Compounds of formula Ib, wherein each of R³ and R² are H, R⁴ is J, R¹ is OCHF₂ and wherein R⁵ and R⁶ are as defined in one row of table A;
Table 356: Compounds of formula Ib, wherein each of R³ and R² are H, R⁴ is J, R¹ is OCClF₂ and wherein R⁵ and R⁶ are as defined in one row of table A;
Table 357: Compounds of formula Ic, wherein each of R³ and R⁴ are H, R² is H, R¹ is CH₃ and wherein R⁵ and R⁶ are as defined in one row of table A;
Table 358: Compounds of formula Ic, wherein each of R³ and R⁴ are H, R² is H, R¹ is C₂H₅ and wherein R⁵ and R⁶ are as defined in one row of table A;
Table 359: Compounds of formula Ic, wherein each of R³ and R⁴ are H, R² is H, R¹ is CF₃ and wherein R⁵ and R⁶ are as defined in one row of table A;
Table 360: Compounds of formula Ic, wherein each of R³ and R⁴ are H, R² is H, R¹ is F and wherein R⁵ and R⁶ are as defined in one row of table A;
Table 361: Compounds of formula Ic, wherein each of R³ and R⁴ are H, R² is H, R¹ is Cl and wherein R⁵ and R⁶ are as defined in one row of table A;
Table 362: Compounds of formula Ic, wherein each of R³ and R⁴ are H, R² is H, R¹ is Br and wherein R⁵ and R⁶ are as defined in one row of table A;
Table 363: Compounds of formula Ic, wherein each of R³ and R⁴ are H, R² is H, R¹ is J and wherein R⁵ and R⁶ are as defined in one row of table A;
Table 364: Compounds of formula Ic, wherein each of R³ and R⁴ are H, R² is H, R¹ is OCH₃ and wherein R⁵ and R⁶ are as defined in one row of table A;
Table 365: Compounds of formula Ic, wherein each of R³ and R⁴ are H, R² is H, R¹ is OC₂H₅ and wherein R⁵ and R⁶ are as defined in one row of table A;
Table 366: Compounds of formula Ic, wherein each of R³ and R⁴ are H, R² is H, R¹ is OCF₃ and wherein R⁵ and R⁶ are as defined in one row of table A;
Table 367: Compounds of formula Ic, wherein each of R³ and R⁴ are H, R² is H, R¹ is OCHF₂ and wherein R⁵ and R⁶ are as defined in one row of table A;
Table 368: Compounds of formula Ic, wherein each of R³ and R⁴ are H, R² is H, R¹ is OCClF₂ and wherein R⁵ and R⁶ are as defined in one row of table A;
Table 369: Compounds of formula Ic, wherein each of R³ and R⁴ are H, R² is F, R¹ is CH₃ and wherein R⁵ and R⁶ are as defined in one row of table A;
Table 370: Compounds of formula Ic, wherein each of R³ and R⁴ are H, R² is F, R¹ is C₂H₅ and wherein R⁵ and R⁶ are as defined in one row of table A;
Table 371: Compounds of formula Ic, wherein each of R³ and R⁴ are H, R² is F, R¹ is CF₃ and wherein R⁵ and R⁶ are as defined in one row of table A;
Table 372: Compounds of formula Ic, wherein each of R³ and R⁴ are H, R² is F, R¹ is F and wherein R⁵ and R⁶ are as defined in one row of table A;
Table 373: Compounds of formula Ic, wherein each of R³ and R⁴ are H, R² is F, R¹ is Cl and wherein R⁵ and R⁶ are as defined in one row of table A;
Table 374: Compounds of formula Ic, wherein each of R³ and R⁴ are H, R² is F, R¹ is Br and wherein R⁵ and R⁶ are as defined in one row of table A;
Table 375: Compounds of formula Ic, wherein each of R³ and R⁴ are H, R² is F, R¹ is J and wherein R⁵ and R⁶ are as defined in one row of table A;
Table 376: Compounds of formula Ic, wherein each of R³ and R⁴ are H, R² is F, R¹ is OCH₃ and wherein R⁵ and R⁶ are as defined in one row of table A;
Table 377: Compounds of formula Ic, wherein each of R³ and R⁴ are H, R² is F, R¹ is OC₂H₅ and wherein R⁵ and R⁶ are as defined in one row of table A;
Table 378: Compounds of formula Ic, wherein each of R³ and R⁴ are H, R² is F, R¹ is OCF₃ and wherein R⁵ and R⁶ are as defined in one row of table A;
Table 379: Compounds of formula Ic, wherein each of R³ and R⁴ are H, R² is F, R¹ is OCHF₂ and wherein R⁵ and R⁶ are as defined in one row of table A;
Table 380: Compounds of formula Ic, wherein each of R³ and R⁴ are H, R² is F, R¹ is OCClF₂ and wherein R⁵ and R⁶ are as defined in one row of table A;
Table 381: Compounds of formula Ic, wherein each of R³ and R⁴ are H, R² is Cl, R¹ is CH₃ and wherein R⁵ and R⁶ are as defined in one row of table A;
Table 382: Compounds of formula Ic, wherein each of R³ and R⁴ are H, R² is Cl, R¹ is C₂H₅ and wherein R⁵ and R⁶ are as defined in one row of table A;
Table 383: Compounds of formula Ic, wherein each of R³ and R⁴ are H, R² is Cl, R¹ is CF₃ and wherein R⁵ and R⁶ are as defined in one row of table A;
Table 384: Compounds of formula Ic, wherein each of R³ and R⁴ are H, R² is Cl, R¹ is F and wherein R⁵ and R⁶ are as defined in one row of table A;
Table 385: Compounds of formula Ic, wherein each of R³ and R⁴ are H, R² is Cl, R¹ is Cl and wherein R⁵ and R⁶ are as defined in one row of table A;
Table 386: Compounds of formula Ic, wherein each of R³ and R⁴ are H, R² is Cl, R¹ is Br and wherein R⁵ and R⁶ are as defined in one row of table A;
Table 387: Compounds of formula Ic, wherein each of R³ and R⁴ are H, R² is Cl, R¹ is J and wherein R⁵ and R⁶ are as defined in one row of table A;
Table 388: Compounds of formula Ic, wherein each of R³ and R⁴ are H, R² is Cl, R¹ is OCH₃ and wherein R⁵ and R⁶ are as defined in one row of table A;
Table 389: Compounds of formula Ic, wherein each of R³ and R⁴ are H, R² is Cl, R¹ is OC₂H₅ and wherein R⁵ and R⁶ are as defined in one row of table A;
Table 390: Compounds of formula Ic, wherein each of R³ and R⁴ are H, R² is Cl, R¹ is OCF₃ and wherein R⁵ and R⁶ are as defined in one row of table A;
Table 391: Compounds of formula Ic, wherein each of R³ and R⁴ are H, R² is Cl, R¹ is OCHF₂ and wherein R⁵ and R⁶ are as defined in one row of table A;
Table 392: Compounds of formula Ic, wherein each of R³ and R⁴ are H, R² is Cl, R¹ is OCClF₂ and wherein R⁵ and R⁶ are as defined in one row of table A;
Table 393: Compounds of formula Ic, wherein each of R³ and R⁴ are H, R² is Br, R¹ is CH₃ and wherein R⁵ and R⁶ are as defined in one row of table A;
Table 394: Compounds of formula Ic, wherein each of R³ and R⁴ are H, R² is Br, R¹ is C₂H₅ and wherein R⁵ and R⁶ are as defined in one row of table A;
Table 395: Compounds of formula Ic, wherein each of R³ and R⁴ are H, R² is Br, R¹ is CF₃ and wherein R⁵ and R⁶ are as defined in one row of table A;
Table 396: Compounds of formula Ic, wherein each of R³ and R⁴ are H, R² is Br, R¹ is F and wherein R⁵ and R⁶ are as defined in one row of table A;
Table 397: Compounds of formula Ic, wherein each of R³ and R⁴ are H, R² is Br, R¹ is Cl and wherein R⁵ and R⁶ are as defined in one row of table A;
Table 398: Compounds of formula Ic, wherein each of R³ and R⁴ are H, R² is Br, R¹ is Br and wherein R⁵ and R⁶ are as defined in one row of table A;
Table 399: Compounds of formula Ic, wherein each of R³ and R⁴ are H, R² is Br, R¹ is J and wherein R⁵ and R⁶ are as defined in one row of table A;
Table 400: Compounds of formula Ic, wherein each of R³ and R⁴ are H, R² is Br, R¹ is OCH₃ and wherein R⁵ and R⁶ are as defined in one row of table A;
Table 401: Compounds of formula Ic, wherein each of R³ and R⁴ are H, R² is Br, R¹ is OC₂H₅ and wherein R⁵ and R⁶ are as defined in one row of table A;
Table 402: Compounds of formula Ic, wherein each of R³ and R⁴ are H, R² is Br, R¹ is OCF₃ and wherein R⁵ and R⁶ are as defined in one row of table A;
Table 403: Compounds of formula Ic, wherein each of R³ and R⁴ are H, R² is Br, R¹ is OCHF₂ and wherein R⁵ and R⁶ are as defined in one row of table A;
Table 404: Compounds of formula Ic, wherein each of R³ and R⁴ are H, R² is Br, R¹ is OCClF₂ and wherein R⁵ and R⁶ are as defined in one row of table A;
Table 405: Compounds of formula Ic, wherein each of R³ and R⁴ are H, R² is J, R¹ is CH₃ and wherein R⁵ and R⁶ are as defined in one row of table A;
Table 406: Compounds of formula Ic, wherein each of R³ and R⁴ are H, R² is J, R¹ is C₂H₅ and wherein R⁵ and R⁶ are as defined in one row of table A;
Table 407: Compounds of formula Ic, wherein each of R³ and R⁴ are H, R² is J, R¹ is CF₃ and wherein R⁵ and R⁶ are as defined in one row of table A;
Table 408: Compounds of formula Ic, wherein each of R³ and R⁴ are H, R² is J, R¹ is F and wherein R⁵ and R⁶ are as defined in one row of table A;
Table 409: Compounds of formula Ic, wherein each of R³ and R⁴ are H, R² is J, R¹ is Cl and wherein R⁵ and R⁶ are as defined in one row of table A;
Table 410: Compounds of formula Ic, wherein each of R³ and R⁴ are H, R² is J, R¹ is Br and wherein R⁵ and R⁶ are as defined in one row of table A;
Table 411: Compounds of formula Ic, wherein each of R³ and R⁴ are H, R² is J, R¹ is J and wherein R⁵ and R⁶ are as defined in one row of table A;
Table 412: Compounds of formula Ic, wherein each of R³ and R⁴ are H, R² is J, R¹ is OCH₃ and wherein R⁵ and R⁶ are as defined in one row of table A;
Table 413: Compounds of formula Ic, wherein each of R³ and R⁴ are H, R² is J, R¹ is OC₂H₅ and wherein R⁵ and R⁶ are as defined in one row of table A;
Table 414: Compounds of formula Ic, wherein each of R³ and R⁴ are H, R² is J, R¹ is OCF₃ and wherein R⁵ and R⁶ are as defined in one row of table A;
Table 415: Compounds of formula Ic, wherein each of R³ and R⁴ are H, R² is J, R¹ is OCHF₂ and wherein R⁵ and R⁶ are as defined in one row of table A;
Table 416: Compounds of formula Ic, wherein each of R³ and R⁴ are H, R² is J, R¹ is OCClF₂ and wherein R⁵ and R⁶ are as defined in one row of table A;
Table 417: Compounds of formula Ic, wherein each of R² and R⁴ are H, R³ is H, R¹ is C₂H₅ and wherein R⁵ and R⁶ are as defined in one row of table A;
Table 418: Compounds of formula Ic, wherein each of R² and R⁴ are H, R³ is H, R¹ is CF₃ and wherein R⁵ and R⁶ are as defined in one row of table A;
Table 419: Compounds of formula Ic, wherein each of R² and R⁴ are H, R³ is H, R¹ is F and wherein R⁵ and R⁶ are as defined in one row of table A;
Table 420: Compounds of formula Ic, wherein each of R² and R⁴ are H, R³ is H, R¹ is Cl and wherein R⁵ and R⁶ are as defined in one row of table A;
Table 421: Compounds of formula Ic, wherein each of R² and R⁴ are H, R³ is H, R¹ is Br and wherein R⁵ and R⁶ are as defined in one row of table A;
Table 422: Compounds of formula Ic, wherein each of R² and R⁴ are H, R³ is H, R¹ is J and wherein R⁵ and R⁶ are as defined in one row of table A;
Table 423: Compounds of formula Ic, wherein each of R² and R⁴ are H, R³ is H, R¹ is OCH₃ and wherein R⁵ and R⁶ are as defined in one row of table A;
Table 424: Compounds of formula Ic, wherein each of R² and R⁴ are H, R³ is H, R¹ is OC₂H₅ and wherein R⁵ and R⁶ are as defined in one row of table A;
Table 425: Compounds of formula Ic, wherein each of R² and R⁴ are H, R³ is H, R¹ is OCF₃ and wherein R⁵ and R⁶ are as defined in one row of table A;
Table 426: Compounds of formula Ic, wherein each of R² and R⁴ are H, R³ is H, R¹ is OCHF₂ and wherein R⁵ and R⁶ are as defined in one row of table A;
Table 427: Compounds of formula Ic, wherein each of R² and R⁴ are H, R³ is H, R¹ is OCClF₂ and wherein R⁵ and R⁶ are as defined in one row of table A;
Table 428: Compounds of formula Ic, wherein each of R² and R⁴ are H, R³ is F, R¹ is CH₃ and wherein R⁵ and R⁶ are as defined in one row of table A;
Table 429: Compounds of formula Ic, wherein each of R² and R⁴ are H, R³ is F, R¹ is C₂H₅ and wherein R⁵ and R⁶ are as defined in one row of table A;
Table 430: Compounds of formula Ic, wherein each of R² and R⁴ are H, R³ is F, R¹ is CF₃ and wherein R⁵ and R⁶ are as defined in one row of table A;
Table 431: Compounds of formula Ic, wherein each of R² and R⁴ are H, R³ is F, R¹ is F and wherein R⁵ and R⁶ are as defined in one row of table A;
Table 432: Compounds of formula Ic, wherein each of R² and R⁴ are H, R³ is F, R¹ is Cl and wherein R⁵ and R⁶ are as defined in one row of table A;
Table 433: Compounds of formula Ic, wherein each of R² and R⁴ are H, R³ is F, R¹ is Br and wherein R⁵ and R⁶ are as defined in one row of table A;
Table 434: Compounds of formula Ic, wherein each of R² and R⁴ are H, R³ is F, R¹ is J and wherein R⁵ and R⁶ are as defined in one row of table A;
Table 435: Compounds of formula Ic, wherein each of R² and R⁴ are H, R³ is F, R¹ is OCH₃ and wherein R⁵ and R⁶ are as defined in one row of table A;
Table 436: Compounds of formula Ic, wherein each of R² and R⁴ are H, R³ is F, R¹ is OC₂H₅ and wherein R⁵ and R⁶ are as defined in one row of table A;
Table 437: Compounds of formula Ic, wherein each of R² and R⁴ are H, R³ is F, R¹ is OCF₃ and wherein R⁵ and R⁶ are as defined in one row of table A;
Table 438: Compounds of formula Ic, wherein each of R² and R⁴ are H, R³ is F, R¹ is OCHF₂ and wherein R⁵ and R⁶ are as defined in one row of table A;
Table 439: Compounds of formula Ic, wherein each of R² and R⁴ are H, R³ is F, R¹ is OCClF₂ and wherein R⁵ and R⁶ are as defined in one row of table A;
Table 440: Compounds of formula Ic, wherein each of R² and R⁴ are H, R³ is Cl, R¹ is CH₃ and wherein R⁵ and R⁶ are as defined in one row of table A;
Table 441: Compounds of formula Ic, wherein each of R² and R⁴ are H, R³ is Cl, R¹ is C₂H₅ and wherein R⁵ and R⁶ are as defined in one row of table A;
Table 442: Compounds of formula Ic, wherein each of R² and R⁴ are H, R³ is Cl, R¹ is CF₃ and wherein R⁵ and R⁶ are as defined in one row of table A;
Table 443: Compounds of formula Ic, wherein each of R² and R⁴ are H, R³ is Cl, R¹ is F and wherein R⁵ and R⁶ are as defined in one row of table A;
Table 444: Compounds of formula Ic, wherein each of R² and R⁴ are H, R³ is Cl, R¹ is Cl and wherein R⁵ and R⁶ are as defined in one row of table A;
Table 445: Compounds of formula Ic, wherein each of R² and R⁴ are H, R³ is Cl, R¹ is Br and wherein R⁵ and R⁶ are as defined in one row of table A;
Table 446: Compounds of formula Ic, wherein each of R² and R⁴ are H, R³ is Cl, R¹ is J and wherein R⁵ and R⁶ are as defined in one row of table A;
Table 447: Compounds of formula Ic, wherein each of R² and R⁴ are H, R³ is Cl, R¹ is OCH₃ and wherein R⁵ and R⁶ are as defined in one row of table A;
Table 448: Compounds of formula Ic, wherein each of R² and R⁴ are H, R³ is Cl, R¹ is OC₂H₅ and wherein R⁵ and R⁶ are as defined in one row of table A;
Table 449: Compounds of formula Ic, wherein each of R² and R⁴ are H, R³ is Cl, R¹ is OCF₃ and wherein R⁵ and R⁶ are as defined in one row of table A;
Table 450: Compounds of formula Ic, wherein each of R² and R⁴ are H, R³ is Cl, R¹ is OCHF₂ and wherein R⁵ and R⁶ are as defined in one row of table A;
Table 451: Compounds of formula Ic, wherein each of R² and R⁴ are H, R³ is Cl, R¹ is OCClF₂ and wherein R⁵ and R⁶ are as defined in one row of table A;
Table 452: Compounds of formula Ic, wherein each of R² and R⁴ are H, R³ is Br, R¹ is CH₃ and wherein R⁵ and R⁶ are as defined in one row of table A;
Table 453: Compounds of formula Ic, wherein each of R² and R⁴ are H, R³ is Br, R¹ is C₂H₅ and wherein R⁵ and R⁶ are as defined in one row of table A;
Table 454: Compounds of formula Ic, wherein each of R² and R⁴ are H, R³ is Br, R¹ is CF₃ and wherein R⁵ and R⁶ are as defined in one row of table A;
Table 455: Compounds of formula Ic, wherein each of R² and R⁴ are H, R³ is Br, R¹ is F and wherein R⁵ and R⁶ are as defined in one row of table A;
Table 456: Compounds of formula Ic, wherein each of R² and R⁴ are H, R³ is Br, R¹ is Cl and wherein R⁵ and R⁶ are as defined in one row of table A;
Table 457: Compounds of formula Ic, wherein each of R² and R⁴ are H, R³ is Br, R¹ is Br and wherein R⁵ and R⁶ are as defined in one row of table A;
Table 458: Compounds of formula Ic, wherein each of R² and R⁴ are H, R³ is Br, R¹ is J and wherein R⁵ and R⁶ are as defined in one row of table A;
Table 459: Compounds of formula Ic, wherein each of R² and R⁴ are H, R³ is Br, R¹ is OCH₃ and wherein R⁵ and R⁶ are as defined in one row of table A;
Table 460: Compounds of formula Ic, wherein each of R² and R⁴ are H, R³ is Br, R¹ is OC₂H₅ and wherein R⁵ and R⁶ are as defined in one row of table A;
Table 461: Compounds of formula Ic, wherein each of R² and R⁴ are H, R³ is Br, R¹ is OCF₃ and wherein R⁵ and R⁶ are as defined in one row of table A;
Table 462: Compounds of formula Ic, wherein each of R² and R⁴ are H, R³ is Br, R¹ is OCHF₂ and wherein R⁵ and R⁶ are as defined in one row of table A;
Table 463: Compounds of formula Ic, wherein each of R² and R⁴ are H, R³ is Br, R¹ is OCClF₂ and wherein R⁵ and R⁶ are as defined in one row of table A;
Table 464: Compounds of formula Ic, wherein each of R² and R⁴ are H, R³ is J, R¹ is CH₃ and wherein R⁵ and R⁶ are as defined in one row of table A;
Table 465: Compounds of formula Ic, wherein each of R² and R⁴ are H, R³ is J, R¹ is C₂H₅ and wherein R⁵ and R⁶ are as defined in one row of table A;
Table 466: Compounds of formula Ic, wherein each of R² and R⁴ are H, R³ is J, R¹ is CF₃ and wherein R⁵ and R⁶ are as defined in one row of table A;
Table 467: Compounds of formula Ic, wherein each of R² and R⁴ are H, R³ is J, R¹ is F and wherein R⁵ and R⁶ are as defined in one row of table A;
Table 468: Compounds of formula Ic, wherein each of R² and R⁴ are H, R³ is J, R¹ is Cl and wherein R⁵ and R⁶ are as defined in one row of table A;
Table 469: Compounds of formula Ic, wherein each of R² and R⁴ are H, R³ is J, R¹ is Br and wherein R⁵ and R⁶ are as defined in one row of table A;
Table 470: Compounds of formula Ic, wherein each of R² and R⁴ are H, R³ is J, R¹ is J and wherein R⁵ and R⁶ are as defined in one row of table A;
Table 471: Compounds of formula Ic, wherein each of R² and R⁴ are H, R³ is J, R¹ is OCH₃ and wherein R⁵ and R⁶ are as defined in one row of table A;
Table 472: Compounds of formula Ic, wherein each of R² and R⁴ are H, R³ is J, R¹ is OC₂H₅ and wherein R⁵ and R⁶ are as defined in one row of table A;
Table 473: Compounds of formula Ic, wherein each of R² and R⁴ are H, R³ is J, R¹ is OCF₃ and wherein R⁵ and R⁶ are as defined in one row of table A;
Table 474: Compounds of formula Ic, wherein each of R² and R⁴ are H, R³ is J, R¹ is OCHF₂ and wherein R⁵ and R⁶ are as defined in one row of table A;
Table 475: Compounds of formula Ic, wherein each of R² and R⁴ are H, R³ is J, R¹ is OCClF₂ and wherein R⁵ and R⁶ are as defined in one row of table A;
Table 476: Compounds of formula Ic, wherein each of R³ and R² are H, R⁴ is H, R¹ is C₂H₅ and wherein R⁵ and R⁶ are as defined in one row of table A;
Table 477: Compounds of formula Ic, wherein each of R³ and R² are H, R⁴ is H, R¹ is CF₃ and wherein R⁵ and R⁶ are as defined in one row of table A;
Table 478: Compounds of formula Ic, wherein each of R³ and R² are H, R⁴ is H, R¹ is F and wherein R⁵ and R⁶ are as defined in one row of table A;
Table 479: Compounds of formula Ic, wherein each of R³ and R² are H, R⁴ is H, R¹ is Cl and wherein R⁵ and R⁶ are as defined in one row of table A;
Table 480: Compounds of formula Ic, wherein each of R³ and R² are H, R⁴ is H, R¹ is Br and wherein R⁵ and R⁶ are as defined in one row of table A;
Table 481: Compounds of formula Ic, wherein each of R³ and R² are H, R⁴ is H, R¹ is J and wherein R⁵ and R⁶ are as defined in one row of table A;
Table 482: Compounds of formula Ic, wherein each of R³ and R² are H, R⁴ is H, R¹ is OCH₃ and wherein R⁵ and R⁶ are as defined in one row of table A;
Table 483: Compounds of formula Ic, wherein each of R³ and R² are H, R⁴ is H, R¹ is OC₂H₅ and wherein R⁵ and R⁶ are as defined in one row of table A;
Table 484: Compounds of formula Ic, wherein each of R³ and R² are H, R⁴ is H, R¹ is OCF₃ and wherein R⁵ and R⁶ are as defined in one row of table A;
Table 485: Compounds of formula Ic, wherein each of R³ and R² are H, R⁴ is H, R¹ is OCHF₂ and wherein R⁵ and R⁶ are as defined in one row of table A;
Table 486: Compounds of formula Ic, wherein each of R³ and R² are H, R⁴ is H, R¹ is OCClF₂ and wherein R⁵ and R⁶ are as defined in one row of table A;
Table 487: Compounds of formula Ic, wherein each of R³ and R² are H, R⁴ is F, R¹ is CH₃ and wherein R⁵ and R⁶ are as defined in one row of table A;
Table 488: Compounds of formula Ic, wherein each of R³ and R² are H, R⁴ is F, R¹ is C₂H₅ and wherein R⁵ and R⁶ are as defined in one row of table A;
Table 489: Compounds of formula Ic, wherein each of R³ and R² are H, R⁴ is F, R¹ is CF₃ and wherein R⁵ and R⁶ are as defined in one row of table A;
Table 490: Compounds of formula Ic, wherein each of R³ and R² are H, R⁴ is F, R¹ is F and wherein R⁵ and R⁶ are as defined in one row of table A;
Table 491: Compounds of formula Ic, wherein each of R³ and R² are H, R⁴ is F, R¹ is Cl and wherein R⁵ and R⁶ are as defined in one row of table A;
Table 492: Compounds of formula Ic, wherein each of R³ and R² are H, R⁴ is F, R¹ is Br and wherein R⁵ and R⁶ are as defined in one row of table A;
Table 493: Compounds of formula Ic, wherein each of R³ and R² are H, R⁴ is F, R¹ is J and wherein R⁵ and R⁶ are as defined in one row of table A;
Table 494: Compounds of formula Ic, wherein each of R³ and R² are H, R⁴ is F, R¹ is OCH₃ and wherein R⁵ and R⁶ are as defined in one row of table A;
Table 495: Compounds of formula Ic, wherein each of R³ and R² are H, R⁴ is F, R¹ is OC₂H₅ and wherein R⁵ and R⁶ are as defined in one row of table A;
Table 496: Compounds of formula Ic, wherein each of R³ and R² are H, R⁴ is F, R¹ is OCF₃ and wherein R⁵ and R⁶ are as defined in one row of table A;
Table 497: Compounds of formula Ic, wherein each of R³ and R² are H, R⁴ is F, R¹ is OCHF₂ and wherein R⁵ and R⁶ are as defined in one row of table A;
Table 498: Compounds of formula Ic, wherein each of R³ and R² are H, R⁴ is F, R¹ is OCClF₂ and wherein R⁵ and R⁶ are as defined in one row of table A;
Table 499: Compounds of formula Ic, wherein each of R³ and R² are H, R⁴ is Cl, R¹ is CH₃ and wherein R⁵ and R⁶ are as defined in one row of table A;
Table 500: Compounds of formula Ic, wherein each of R³ and R² are H, R⁴ is Cl, R¹ is C₂H₅ and wherein R⁵ and R⁶ are as defined in one row of table A;
Table 501: Compounds of formula Ic, wherein each of R³ and R² are H, R⁴ is Cl, R¹ is CF₃ and wherein R⁵ and R⁶ are as defined in one row of table A;
Table 502: Compounds of formula Ic, wherein each of R³ and R² are H, R⁴ is Cl, R¹ is F and wherein R⁵ and R⁶ are as defined in one row of table A;
Table 503: Compounds of formula Ic, wherein each of R³ and R² are H, R⁴ is Cl, R¹ is Cl and wherein R⁵ and R⁶ are as defined in one row of table A;
Table 504: Compounds of formula Ic, wherein each of R³ and R² are H, R⁴ is Cl, R¹ is Br and wherein R⁵ and R⁶ are as defined in one row of table A;
Table 505: Compounds of formula Ic, wherein each of R³ and R² are H, R⁴ is Cl, R¹ is J and wherein R⁵ and R⁶ are as defined in one row of table A;
Table 506: Compounds of formula Ic, wherein each of R³ and R² are H, R⁴ is Cl, R¹ is OCH₃ and wherein R⁵ and R⁶ are as defined in one row of table A;
Table 507: Compounds of formula Ic, wherein each of R³ and R² are H, R⁴ is Cl, R¹ is OC₂H₅ and wherein R⁵ and R⁶ are as defined in one row of table A;
Table 508: Compounds of formula Ic, wherein each of R³ and R² are H, R⁴ is Cl, R¹ is OCF₃ and wherein R⁵ and R⁶ are as defined in one row of table A;
Table 509: Compounds of formula Ic, wherein each of R³ and R² are H, R⁴ is Cl, R¹ is OCHF₂ and wherein R⁵ and R⁶ are as defined in one row of table A;
Table 510: Compounds of formula Ic, wherein each of R³ and R² are H, R⁴ is Cl, R¹ is OCClF₂ and wherein R⁵ and R⁶ are as defined in one row of table A;
Table 511: Compounds of formula Ic, wherein each of R³ and R² are H, R⁴ is Br, R¹ is CH₃ and wherein R⁵ and R⁶ are as defined in one row of table A;
Table 512: Compounds of formula Ic, wherein each of R³ and R² are H, R⁴ is Br, R¹ is C₂H₅ and wherein R⁵ and R⁶ are as defined in one row of table A;
Table 513: Compounds of formula Ic, wherein each of R³ and R² are H, R⁴ is Br, R¹ is CF₃ and wherein R⁵ and R⁶ are as defined in one row of table A;
Table 514: Compounds of formula Ic, wherein each of R³ and R² are H, R⁴ is Br, R¹ is F and wherein R⁵ and R⁶ are as defined in one row of table A;
Table 515: Compounds of formula Ic, wherein each of R³ and R² are H, R⁴ is Br, R¹ is Cl and wherein R⁵ and R⁶ are as defined in one row of table A;
Table 516: Compounds of formula Ic, wherein each of R³ and R² are H, R⁴ is Br, R¹ is Br and wherein R⁵ and R⁶ are as defined in one row of table A;
Table 517: Compounds of formula Ic, wherein each of R³ and R² are H, R⁴ is Br, R¹ is J and wherein R⁵ and R⁶ are as defined in one row of table A;
Table 518: Compounds of formula Ic, wherein each of R³ and R² are H, R⁴ is Br, R¹ is OCH₃ and wherein R⁵ and R⁶ are as defined in one row of table A;
Table 519: Compounds of formula Ic, wherein each of R³ and R² are H, R⁴ is Br, R¹ is OC₂H₅ and wherein R⁵ and R⁶ are as defined in one row of table A;
Table 520: Compounds of formula Ic, wherein each of R³ and R² are H, R⁴ is Br, R¹ is OCF₃ and wherein R⁵ and R⁶ are as defined in one row of table A;
Table 521: Compounds of formula Ic, wherein each of R³ and R² are H, R⁴ is Br, R¹ is OCHF₂ and wherein R⁵ and R⁶ are as defined in one row of table A;
Table 522: Compounds of formula Ic, wherein each of R³ and R² are H, R⁴ is Br, R¹ is OCClF₂ and wherein R⁵ and R⁶ are as defined in one row of table A;
Table 523: Compounds of formula Ic, wherein each of R³ and R² are H, R⁴ is J, R¹ is CH₃ and wherein R⁵ and R⁶ are as defined in one row of table A;
Table 524: Compounds of formula Ic, wherein each of R³ and R² are H, R⁴ is J, R¹ is C₂H₅ and wherein R⁵ and R⁶ are as defined in one row of table A;
Table 525: Compounds of formula Ic, wherein each of R³ and R² are H, R⁴ is J, R¹ is CF₃ and wherein R⁵ and R⁶ are as defined in one row of table A;
Table 526: Compounds of formula Ic, wherein each of R³ and R² are H, R⁴ is J, R¹ is F and wherein R⁵ and R⁶ are as defined in one row of table A;
Table 527: Compounds of formula Ic, wherein each of R³ and R² are H, R⁴ is J, R¹ is Cl and wherein R⁵ and R⁶ are as defined in one row of table A;
Table 528: Compounds of formula Ic, wherein each of R³ and R² are H, R⁴ is J, R¹ is Br and wherein R⁵ and R⁶ are as defined in one row of table A;
Table 529: Compounds of formula Ic, wherein each of R³ and R² are H, R⁴ is J, R¹ is J and wherein R⁵ and R⁶ are as defined in one row of table A;
Table 530: Compounds of formula Ic, wherein each of R³ and R² are H, R⁴ is J, R¹ is OCH₃ and wherein R⁵ and R⁶ are as defined in one row of table A;
Table 531: Compounds of formula Ic, wherein each of R³ and R² are H, R⁴ is J, R¹ is OC₂H₅ and wherein R⁵ and R⁶ are as defined in one row of table A;
Table 532: Compounds of formula Ic, wherein each of R³ and R² are H, R⁴ is J, R¹ is OCF₃ and wherein R⁵ and R⁶ are as defined in one row of table A;
Table 533: Compounds of formula Ic, wherein each of R³ and R² are H, R⁴ is J, R¹ is OCHF₂ and wherein R⁵ and R⁶ are as defined in one row of table A;
Table 534: Compounds of formula Ic, wherein each of R³ and R² are H, R⁴ is J, R¹ is OCClF₂ and wherein R⁵ and R⁶ are as defined in one row of table A;

Table A referring to formulae Ia, IP and Ic:

| | R⁵ | R⁶ |
|---|---|---|
| A.1 | CH₃ | CH₃ |
| A.2 | CH₃ | H₃C—CH₂ |
| A.3 | CH₃ | H₂C═CH |
| A.4 | CH₃ | H₃C—O—CH₂ |
| A.5 | CH₃ | ClCH₂—CH₂ |
| A.6 | CH₃ | FCH₂—CH₂ |
| A.7 | CH₃ | F₃C—CH₂ |
| A.8 | CH₃ | Cl₃C—CH₂ |
| A.9 | CH₃ | HO—CH₂—CH₂ |
| A.10 | CH₃ | H₃C—O—CH₂—CH₂ |
| A.11 | CH₃ | (CH₃)₂CH |
| A.12 | CH₃ | H₃C—CH₂—CH₂ |
| A.13 | CH₃ | CN—CH₂ |
| A.14 | CH₃ | CN—CH₂—CH₂ |
| A.15 | CH₃ | F₃C—CH₂—CH₂ |
| A.16 | CH₃ | Cl₃C—CH₂—CH₂ |
| A.17 | CH₃ | H₃C—CH₂—CH₂—CH₂ |
| A.18 | CH₃ | H₃C—CH₂—CH(CH₃) |
| A.19 | CH₃ | H₃C—CH(CH₃)—CH₂ |
| A.20 | CH₃ | H₂C═C(CH₃)—CH₂ |
| A.21 | CH₃ | HC≡C—CH(CH₃) |
| A.22 | CH₃ | HC≡C—CH₂ |
| A.23 | CH₃ | (CH₃)₃C |
| A.24 | CH₃ | HO |
| A.25 | CH₃ | H₃C—O |
| A.26 | CH₃ | H₃C—CH₂—O |
| A.27 | CH₃ | (CH₃)₂CH—O |
| A.28 | CH₃ | H₃C—CH₂—CH₂—O |
| A.29 | CH₃ | H₂C═CH—CH₂—O |
| A.30 | CH₃ | H₂C═CH—CH₂ |
| A.31 | CH₃ | HC≡C—CH₂—O |
| A.32 | CH₃ | (CH₃)₃C—O |
| A.33 | CH₃ | (CH₃)₂CH—CH₂—O |
| A.34 | CH₃ | (CH₃O)₂C(CH₃) |
| A.35 | CH₃ | (CH₃O)₂CH |
| A.36 | CH₃ | (C₆H₅)—CH₂ |
| A.37 | CH₃ | cyclopropyl |
| A.38 | H₃C—CH₂ | CH₃ |
| A.39 | H₃C—CH₂ | H₃C—CH₂ |
| A.40 | H₃C—CH₂ | H₂C═CH |
| A.41 | H₃C—CH₂ | H₃C—O—CH₂ |
| A.42 | H₃C—CH₂ | ClCH₂—CH₂ |
| A.43 | H₃C—CH₂ | FCH₂—CH₂ |
| A.44 | H₃C—CH₂ | F₃C—CH₂ |
| A.45 | H₃C—CH₂ | Cl₃C—CH₂ |
| A.46 | H₃C—CH₂ | HO—CH₂—CH₂ |
| A.47 | H₃C—CH₂ | H₃C—O—CH₂—CH₂ |
| A.48 | H₃C—CH₂ | (CH₃)₂CH |
| A.49 | H₃C—CH₂ | H₃C—CH₂—CH₂ |
| A.50 | H₃C—CH₂ | CN—CH₂ |
| A.51 | H₃C—CH₂ | CN—CH₂—CH₂ |
| A.52 | H₃C—CH₂ | F₃C—CH₂—CH₂ |
| A.53 | H₃C—CH₂ | Cl₃C—CH₂—CH₂ |
| A.54 | H₃C—CH₂ | H₃C—CH₂—CH₂—CH₂ |
| A.55 | H₃C—CH₂ | H₃C—CH₂—CH(CH₃) |
| A.56 | H₃C—CH₂ | H₃C—CH(CH₃)—CH₂ |
| A.57 | H₃C—CH₂ | H₂C═C(CH₃)—CH₂ |
| A.58 | H₃C—CH₂ | HC≡C—CH(CH₃) |
| A.59 | H₃C—CH₂ | HC≡C—CH₂ |
| A.60 | H₃C—CH₂ | (CH₃)₃C |
| A.61 | H₃C—CH₂ | HO |
| A.62 | H₃C—CH₂ | H₃C—O |
| A.63 | H₃C—CH₂ | H₃C—CH₂—O |
| A.64 | H₃C—CH₂ | (CH₃)₂CH—O |
| A.65 | H₃C—CH₂ | H₃C—CH₂—CH₂—O |
| A.66 | H₃C—CH₂ | H₂C═CH—CH₂—O |
| A.67 | H₃C—CH₂ | H₂C═CH—CH₂ |
| A.68 | H₃C—CH₂ | HC≡C—CH₂—O |
| A.69 | H₃C—CH₂ | (CH₃)₃C—O |
| A.70 | H₃C—CH₂ | (CH₃)₂CH—CH₂—O |
| A.71 | H₃C—CH₂ | (CH₃O)₂C(CH₃) |
| A.72 | H₃C—CH₂ | (CH₃O)₂CH |
| A.73 | H₃C—CH₂ | (C₆H₅)—CH₂ |
| A.74 | H₃C—CH₂ | cyclopropyl |
| A.75 | H₃C—CH₂—CH₂ | CH₃ |
| A.76 | H₃C—CH₂—CH₂ | H₃C—CH₂ |
| A.77 | H₃C—CH₂—CH₂ | H₂C═CH |
| A.78 | H₃C—CH₂—CH₂ | H₃C—O—CH₂ |
| A.79 | H₃C—CH₂—CH₂ | ClCH₂—CH₂ |
| A.80 | H₃C—CH₂—CH₂ | FCH₂—CH₂ |
| A.81 | H₃C—CH₂—CH₂ | F₃C—CH₂ |
| A.82 | H₃C—CH₂—CH₂ | Cl₃C—CH₂ |
| A.83 | H₃C—CH₂—CH₂ | HO—CH₂—CH₂ |
| A.84 | H₃C—CH₂—CH₂ | H₃C—O—CH₂—CH₂ |
| A.85 | H₃C—CH₂—CH₂ | (CH₃)₂CH |
| A.86 | H₃C—CH₂—CH₂ | H₃C—CH₂—CH₂ |
| A.87 | H₃C—CH₂—CH₂ | CN—CH₂ |
| A.88 | H₃C—CH₂—CH₂ | CN—CH₂—CH₂ |
| A.89 | H₃C—CH₂—CH₂ | F₃C—CH₂—CH₂ |
| A.90 | H₃C—CH₂—CH₂ | Cl₃C—CH₂—CH₂ |
| A.91 | H₃C—CH₂—CH₂ | H₃C—CH₂—CH₂—CH₂ |
| A.92 | H₃C—CH₂—CH₂ | H₃C—CH₂—CH(CH₃) |
| A.93 | H₃C—CH₂—CH₂ | H₃C—CH(CH₃)—CH₂ |
| A.94 | H₃C—CH₂—CH₂ | H₂C═C(CH₃)—CH₂ |
| A.95 | H₃C—CH₂—CH₂ | HC≡C—CH(CH₃) |
| A.96 | H₃C—CH₂—CH₂ | HC≡C—CH₂ |
| A.97 | H₃C—CH₂—CH₂ | (CH₃)₃C |
| A.98 | H₃C—CH₂—CH₂ | HO |
| A.99 | H₃C—CH₂—CH₂ | H₃C—O |
| A.100 | H₃C—CH₂—CH₂ | H₃C—CH₂—O |
| A.101 | H₃C—CH₂—CH₂ | (CH₃)₂CH—O |
| A.102 | H₃C—CH₂—CH₂ | H₃C—CH₂—CH₂—O |
| A.103 | H₃C—CH₂—CH₂ | H₂C═CH—CH₂—O |
| A.104 | H₃C—CH₂—CH₂ | H₂C═CH—CH₂ |
| A.105 | H₃C—CH₂—CH₂ | HC≡C—CH₂—O |
| A.106 | H₃C—CH₂—CH₂ | (CH₃)₃C—O |
| A.107 | H₃C—CH₂—CH₂ | (CH₃)₂CH—CH₂—O |
| A.108 | H₃C—CH₂—CH₂ | (CH₃O)₂C(CH₃) |
| A.109 | H₃C—CH₂—CH₂ | (CH₃O)₂CH |
| A.110 | H₃C—CH₂—CH₂ | (C₆H₅)—CH₂ |
| A.111 | H₃C—CH₂—CH₂ | cyclopropyl |
| A.112 | (CH₃)₂CH | CH₃ |
| A.113 | (CH₃)₂CH | H₃C—CH₂ |
| A.114 | (CH₃)₂CH | H₂C═CH |
| A.115 | (CH₃)₂CH | H₃C—O—CH₂ |
| A.116 | (CH₃)₂CH | ClCH₂—CH₂ |
| A.117 | (CH₃)₂CH | FCH₂—CH₂ |
| A.118 | (CH₃)₂CH | F₃C—CH₂ |
| A.119 | (CH₃)₂CH | Cl₃C—CH₂ |
| A.120 | (CH₃)₂CH | HO—CH₂—CH₂ |
| A.121 | (CH₃)₂CH | H₃C—O—CH₂—CH₂ |
| A.122 | (CH₃)₂CH | (CH₃)₂CH |
| A.123 | (CH₃)₂CH | H₃C—CH₂—CH₂ |
| A.124 | (CH₃)₂CH | CN—CH₂ |
| A.125 | (CH₃)₂CH | CN—CH₂—CH₂ |
| A.126 | (CH₃)₂CH | F₃C—CH₂—CH₂ |
| A.127 | (CH₃)₂CH | Cl₃C—CH₂—CH₂ |
| A.128 | (CH₃)₂CH | H₃C—CH₂—CH₂—CH₂ |
| A.129 | (CH₃)₂CH | H₃C—CH₂—CH(CH₃) |
| A.130 | (CH₃)₂CH | H₃C—CH(CH₃)—CH₂ |
| A.131 | (CH₃)₂CH | H₂C═C(CH₃)—CH₂ |
| A.132 | (CH₃)₂CH | HC≡C—CH(CH₃) |
| A.133 | (CH₃)₂CH | HC≡C—CH₂ |
| A.134 | (CH₃)₂CH | (CH₃)₃C |
| A.135 | (CH₃)₂CH | HO |
| A.136 | (CH₃)₂CH | H₃C—O |
| A.137 | (CH₃)₂CH | H₃C—CH₂—O |
| A.138 | (CH₃)₂CH | (CH₃)₂CH—O |
| A.139 | (CH₃)₂CH | H₃C—CH₂—CH₂—O |
| A.140 | (CH₃)₂CH | H₂C═CH—CH₂—O |
| A.141 | (CH₃)₂CH | H₂C═CH—CH₂ |
| A.142 | (CH₃)₂CH | HC≡C—CH₂—O |
| A.143 | (CH₃)₂CH | (CH₃)₃C—O |
| A.144 | (CH₃)₂CH | (CH₃)₂CH—CH₂—O |
| A.145 | (CH₃)₂CH | (CH₃O)₂C(CH₃) |
| A.146 | (CH₃)₂CH | (CH₃O)₂CH |
| A.147 | (CH₃)₂CH | (C₆H₅)—CH₂ |
| A.148 | (CH₃)₂CH | cyclopropyl |
| A.149 | CNCH₂ | CH₃ |
| A.150 | CNCH₂ | H₃C—CH₂ |
| A.151 | CNCH₂ | H₂C═CH |
| A.152 | CNCH₂ | H₃C—O—CH₂ |
| A.153 | CNCH₂ | ClCH₂—CH₂ |
| A.154 | CNCH₂ | FCH₂—CH₂ |
| A.155 | CNCH₂ | F₃C—CH₂ |
| A.156 | CNCH₂ | Cl₃C—CH₂ |
| A.157 | CNCH₂ | HO—CH₂—CH₂ |
| A.158 | CNCH₂ | H₃C—O—CH₂—CH₂ |
| A.159 | CNCH₂ | (CH₃)₂CH |
| A.160 | CNCH₂ | H₃C—CH₂—CH₂ |
| A.161 | CNCH₂ | CN—CH₂ |
| A.162 | CNCH₂ | CN—CH₂—CH₂ |
| A.163 | CNCH₂ | F₃C—CH₂—CH₂ |
| A.164 | CNCH₂ | Cl₃C—CH₂—CH₂ |
| A.165 | CNCH₂ | H₃C—CH₂—CH₂—CH₂ |
| A.166 | CNCH₂ | H₃C—CH₂—CH(CH₃) |
| A.167 | CNCH₂ | H₃C—CH(CH₃)—CH₂ |
| A.168 | CNCH₂ | H₂C═C(CH₃)—CH₂ |
| A.169 | CNCH₂ | HC≡C—CH(CH₃) |
| A.170 | CNCH₂ | HC≡C—CH₂ |
| A.171 | CNCH₂ | (CH₃)₃C |
| A.172 | CNCH₂ | HO |
| A.173 | CNCH₂ | H₃C—O |
| A.174 | CNCH₂ | H₃C—CH₂—O |
| A.175 | CNCH₂ | (CH₃)₂CH—O |
| A.176 | CNCH₂ | H₃C—CH₂—CH₂—O |
| A.177 | CNCH₂ | H₂C═CH—CH₂—O |
| A.178 | CNCH₂ | H₂C═CH—CH₂ |
| A.179 | CNCH₂ | HC≡C—CH₂—O |
| A.180 | CNCH₂ | (CH₃)₃C—O |
| A.181 | CNCH₂ | (CH₃)₂CH—CH₂—O |
| A.182 | CNCH₂ | (CH₃O)₂C(CH₃) |
| A.183 | CNCH₂ | (CH₃O)₂CH |
| A.184 | CNCH₂ | (C₆H₅)—CH₂ |
| A.185 | CNCH₂ | cyclopropyl |
| A.186 | F₃C—CH₂ | CH₃ |
| A.187 | F₃C—CH₂ | H₃C—CH₂ |
| A.188 | F₃C—CH₂ | H₂C═CH |
| A.189 | F₃C—CH₂ | H₃C—O—CH₂ |
| A.190 | F₃C—CH₂ | ClCH₂—CH₂ |
| A.191 | F₃C—CH₂ | FCH₂—CH₂ |
| A.192 | F₃C—CH₂ | F₃C—CH₂ |
| A.193 | F₃C—CH₂ | Cl₃C—CH₂ |
| A.194 | F₃C—CH₂ | HO—CH₂—CH₂ |
| A.195 | F₃C—CH₂ | H₃C—O—CH₂—CH₂ |
| A.196 | F₃C—CH₂ | (CH₃)₂CH |
| A.197 | F₃C—CH₂ | H₃C—CH₂—CH₂ |
| A.198 | F₃C—CH₂ | CN—CH₂ |
| A.199 | F₃C—CH₂ | CN—CH₂—CH₂ |
| A.200 | F₃C—CH₂ | F₃C—CH₂—CH₂ |
| A.201 | F₃C—CH₂ | Cl₃C—CH₂—CH₂ |
| A.202 | F₃C—CH₂ | H₃C—CH₂—CH₂—CH₂ |
| A.203 | F₃C—CH₂ | H₃C—CH₂—CH(CH₃) |
| A.204 | F₃C—CH₂ | H₃C—CH(CH₃)—CH₂ |
| A.205 | F₃C—CH₂ | H₂C═C(CH₃)—CH₂ |
| A.206 | F₃C—CH₂ | HC≡C—CH(CH₃) |
| A.207 | F₃C—CH₂ | HC≡C—CH₂ |
| A.208 | F₃C—CH₂ | (CH₃)₃C |
| A.209 | F₃C—CH₂ | HO |
| A.210 | F₃C—CH₂ | H₃C—O |
| A.211 | F₃C—CH₂ | H₃C—CH₂—O |
| A.212 | F₃C—CH₂ | (CH₃)₂CH—O |
| A.213 | F₃C—CH₂ | H₃C—CH₂—CH₂—O |
| A.214 | F₃C—CH₂ | H₂C═CH—CH₂—O |
| A.215 | F₃C—CH₂ | H₂C═CH—CH₂ |
| A.216 | F₃C—CH₂ | HC≡C—CH₂—O |
| A.217 | F₃C—CH₂ | (CH₃)₃C—O |
| A.218 | F₃C—CH₂ | (CH₃)₂CH—CH₂—O |
| A.219 | F₃C—CH₂ | (CH₃O)₂C(CH₃) |
| A.220 | F₃C—CH₂ | (CH₃O)₂CH |
| A.221 | F₃C—CH₂ | (C₆H₅)—CH₂ |
| A.222 | F₃C—CH₂ | cyclopropyl |
| A.223 | H₃C—CH₂—CH(CH₃) | CH₃ |
| A.224 | H₃C—CH₂—CH(CH₃) | H₃C—CH₂ |
| A.225 | H₃C—CH₂—CH(CH₃) | H₂C═CH |
| A.226 | H₃C—CH₂—CH(CH₃) | H₃C—O—CH₂ |
| A.227 | H₃C—CH₂—CH(CH₃) | ClCH₂—CH₂ |
| A.228 | H₃C—CH₂—CH(CH₃) | FCH₂—CH₂ |
| A.229 | H₃C—CH₂—CH(CH₃) | F₃C—CH₂ |
| A.230 | H₃C—CH₂—CH(CH₃) | Cl₃C—CH₂ |
| A.231 | H₃C—CH₂—CH(CH₃) | HO—CH₂—CH₂ |
| A.232 | H₃C—CH₂—CH(CH₃) | H₃C—O—CH₂—CH₂ |
| A.233 | H₃C—CH₂—CH(CH₃) | (CH₃)₂CH |
| A.234 | H₃C—CH₂—CH(CH₃) | H₃C—CH₂—CH₂ |
| A.235 | H₃C—CH₂—CH(CH₃) | CN—CH₂ |
| A.236 | H₃C—CH₂—CH(CH₃) | CN—CH₂—CH₂ |
| A.237 | H₃C—CH₂—CH(CH₃) | F₃C—CH₂—CH₂ |
| A.238 | H₃C—CH₂—CH(CH₃) | Cl₃C—CH₂—CH₂ |
| A.239 | H₃C—CH₂—CH(CH₃) | H₃C—CH₂—CH₂—CH₂ |
| A.240 | H₃C—CH₂—CH(CH₃) | H₃C—CH₂—CH(CH₃) |
| A.241 | H₃C—CH₂—CH(CH₃) | H₃C—CH(CH₃)—CH₂ |
| A.242 | H₃C—CH₂—CH(CH₃) | H₂C═C(CH₃)—CH₂ |
| A.243 | H₃C—CH₂—CH(CH₃) | HC≡C—CH(CH₃) |
| A.244 | H₃C—CH₂—CH(CH₃) | HC≡C—CH₂ |
| A.245 | H₃C—CH₂—CH(CH₃) | (CH₃)₃C |
| A.246 | H₃C—CH₂—CH(CH₃) | HO |
| A.247 | H₃C—CH₂—CH(CH₃) | H₃C—O |
| A.248 | H₃C—CH₂—CH(CH₃) | H₃C—CH₂—O |
| A.249 | H₃C—CH₂—CH(CH₃) | (CH₃)₂CH—O |
| A.250 | H₃C—CH₂—CH(CH₃) | H₃C—CH₂—CH₂—O |
| A.251 | H₃C—CH₂—CH(CH₃) | H₂C═CH—CH₂—O |
| A.252 | H₃C—CH₂—CH(CH₃) | H₂C═CH—CH₂ |
| A.253 | H₃C—CH₂—CH(CH₃) | HC≡C—CH₂—O |
| A.254 | H₃C—CH₂—CH(CH₃) | (CH₃)₃C—O |
| A.255 | H₃C—CH₂—CH(CH₃) | (CH₃)₂CH—CH₂—O |
| A.256 | H₃C—CH₂—CH(CH₃) | (CH₃O)₂C(CH₃) |
| A.257 | H₃C—CH₂—CH(CH₃) | (CH₃O)₂CH |
| A.258 | H₃C—CH₂—CH(CH₃) | (C₆H₅)—CH₂ |
| A.259 | H₃C—CH₂—CH(CH₃) | cyclopropyl |
| A.260 | (CH₃)₂CH—CH₂ | CH₃ |
| A.261 | (CH₃)₂CH—CH₂ | H₃C—CH₂ |
| A.262 | (CH₃)₂CH—CH₂ | H₂C═CH |
| A.263 | (CH₃)₂CH—CH₂ | H₃C—O—CH₂ |
| A.264 | (CH₃)₂CH—CH₂ | ClCH₂—CH₂ |
| A.265 | (CH₃)₂CH—CH₂ | FCH₂—CH₂ |
| A.266 | (CH₃)₂CH—CH₂ | F₃C—CH₂ |
| A.267 | (CH₃)₂CH—CH₂ | Cl₃C—CH₂ |
| A.268 | (CH₃)₂CH—CH₂ | HO—CH₂—CH₂ |
| A.269 | (CH₃)₂CH—CH₂ | H₃C—O—CH₂—CH₂ |
| A.270 | (CH₃)₂CH—CH₂ | (CH₃)₂CH |
| A.271 | (CH₃)₂CH—CH₂ | H₃C—CH₂—CH₂ |
| A.272 | (CH₃)₂CH—CH₂ | CN—CH₂ |
| A.273 | (CH₃)₂CH—CH₂ | CN—CH₂—CH₂ |
| A.274 | (CH₃)₂CH—CH₂ | F₃C—CH₂—CH₂ |
| A.275 | (CH₃)₂CH—CH₂ | Cl₃C—CH₂—CH₂ |
| A.276 | (CH₃)₂CH—CH₂ | H₃C—CH₂—CH₂—CH₂ |
| A.277 | (CH₃)₂CH—CH₂ | H₃C—CH₂—CH(CH₃) |
| A.278 | (CH₃)₂CH—CH₂ | H₃C—CH(CH₃)—CH₂ |
| A.279 | (CH₃)₂CH—CH₂ | H₂C═C(CH₃)—CH₂ |
| A.280 | (CH₃)₂CH—CH₂ | HC≡C—CH(CH₃) |
| A.281 | (CH₃)₂CH—CH₂ | HC≡C—CH₂ |
| A.282 | (CH₃)₂CH—CH₂ | (CH₃)₃C |
| A.283 | (CH₃)₂CH—CH₂ | HO |
| A.284 | (CH₃)₂CH—CH₂ | H₃C—O |
| A.285 | (CH₃)₂CH—CH₂ | H₃C—CH₂—O |
| A.286 | (CH₃)₂CH—CH₂ | (CH₃)₂CH—O |
| A.287 | (CH₃)₂CH—CH₂ | H₃C—CH₂—CH₂—O |
| A.288 | (CH₃)₂CH—CH₂ | H₂C═CH—CH₂—O |
| A.289 | (CH₃)₂CH—CH₂ | H₂C═CH—CH₂ |
| A.290 | (CH₃)₂CH—CH₂ | HC≡C—CH₂—O |
| A.291 | (CH₃)₂CH—CH₂ | (CH₃)₃C—O |
| A.292 | (CH₃)₂CH—CH₂ | (CH₃)₂CH—CH₂—O |
| A.293 | (CH₃)₂CH—CH₂ | (CH₃O)₂C(CH₃) |
| A.294 | (CH₃)₂CH—CH₂ | (CH₃O)₂CH |
| A.295 | (CH₃)₂CH—CH₂ | (C₆H₅)—CH₂ |
| A.296 | (CH₃)₂CH—CH₂ | cyclopropyl |
| A.297 | H₂C═CH—CH₂ | CH₃ |
| A.298 | H₂C═CH—CH₂ | H₃C—CH₂ |
| A.299 | H₂C═CH—CH₂ | H₂C═CH |
| A.300 | H₂C═CH—CH₂ | H₃C—O—CH₂ |
| A.301 | H₂C═CH—CH₂ | ClCH₂—CH₂ |
| A.302 | H₂C═CH—CH₂ | FCH₂—CH₂ |
| A.303 | H₂C═CH—CH₂ | F₃C—CH₂ |
| A.304 | H₂C═CH—CH₂ | Cl₃C—CH₂ |
| A.305 | H₂C═CH—CH₂ | HO—CH₂—CH₂ |
| A.306 | H₂C═CH—CH₂ | H₃C—O—CH₂—CH₂ |
| A.307 | H₂C═CH—CH₂ | (CH₃)₂CH |
| A.308 | H₂C═CH—CH₂ | H₃C—CH₂—CH₂ |
| A.309 | H₂C═CH—CH₂ | CN—CH₂ |
| A.310 | H₂C═CH—CH₂ | CN—CH₂—CH₂ |
| A.311 | H₂C═CH—CH₂ | F₃C—CH₂—CH₂ |
| A.312 | H₂C═CH—CH₂ | Cl₃C—CH₂—CH₂ |
| A.313 | H₂C═CH—CH₂ | H₃C—CH₂—CH₂—CH₂ |
| A.314 | H₂C═CH—CH₂ | H₃C—CH₂—CH(CH₃) |
| A.315 | H₂C═CH—CH₂ | H₃C—CH(CH₃)—CH₂ |
| A.316 | H₂C═CH—CH₂ | H₂C═C(CH₃)—CH₂ |
| A.317 | H₂C═CH—CH₂ | HC≡C—CH(CH₃) |
| A.318 | H₂C═CH—CH₂ | HC≡C—CH₂ |
| A.319 | H₂C═CH—CH₂ | (CH₃)₃C |
| A.320 | H₂C═CH—CH₂ | HO |
| A.321 | H₂C═CH—CH₂ | H₃C—O |
| A.322 | H₂C═CH—CH₂ | H₃C—CH₂—O |
| A.323 | H₂C═CH—CH₂ | (CH₃)₂CH—O |
| A.324 | H₂C═CH—CH₂ | H₃C—CH₂—CH₂—O |
| A.325 | H₂C═CH—CH₂ | H₂C═CH—CH₂—O |
| A.326 | H₂C═CH—CH₂ | H₂C═CH—CH₂ |
| A.327 | H₂C═CH—CH₂ | HC≡C—CH₂—O |
| A.328 | H₂C═CH—CH₂ | (CH₃)₃C—O |
| A.329 | H₂C═CH—CH₂ | (CH₃)₂CH—CH₂—O |
| A.330 | H₂C═CH—CH₂ | (CH₃O)₂C(CH₃) |
| A.331 | H₂C═CH—CH₂ | (CH₃O)₂CH |
| A.332 | H₂C═CH—CH₂ | (C₆H₅)—CH₂ |
| A.333 | H₂C═CH—CH₂ | cyclopropyl |
| A.334 | HC≡C—CH₂ | CH₃ |
| A.335 | HC≡C—CH₂ | H₃C—CH₂ |
| A.336 | HC≡C—CH₂ | H₂C═CH |
| A.337 | HC≡C—CH₂ | H₃C—O—CH₂ |
| A.338 | HC≡C—CH₂ | ClCH₂—CH₂ |
| A.339 | HC≡C—CH₂ | FCH₂—CH₂ |
| A.340 | HC≡C—CH₂ | F₃C—CH₂ |
| A.341 | HC≡C—CH₂ | Cl₃C—CH₂ |
| A.342 | HC≡C—CH₂ | HO—CH₂—CH₂ |
| A.343 | HC≡C—CH₂ | H₃C—O—CH₂—CH₂ |
| A.344 | HC≡C—CH₂ | (CH₃)₂CH |
| A.345 | HC≡C—CH₂ | H₃C—CH₂—CH₂ |
| A.346 | HC≡C—CH₂ | CN—CH₂ |
| A.347 | HC≡C—CH₂ | CN—CH₂—CH₂ |
| A.348 | HC≡C—CH₂ | F₃C—CH₂—CH₂ |
| A.349 | HC≡C—CH₂ | Cl₃C—CH₂—CH₂ |
| A.350 | HC≡C—CH₂ | H₃C—CH₂—CH₂—CH₂ |
| A.351 | HC≡C—CH₂ | H₃C—CH₂—CH(CH₃) |
| A.352 | HC≡C—CH₂ | H₃C—CH(CH₃)—CH₂ |
| A.353 | HC≡C—CH₂ | H₂C═C(CH₃)—CH₂ |
| A.354 | HC≡C—CH₂ | HC≡C—CH(CH₃) |
| A.355 | HC≡C—CH₂ | HC≡C—CH₂ |
| A.356 | HC≡C—CH₂ | (CH₃)₃C |
| A.357 | HC≡C—CH₂ | HO |
| A.358 | HC≡C—CH₂ | H₃C—O |
| A.359 | HC≡C—CH₂ | H₃C—CH₂—O |
| A.360 | HC≡C—CH₂ | (CH₃)₂CH—O |
| A.361 | HC≡C—CH₂ | H₃C—CH₂—CH₂—O |
| A.362 | HC≡C—CH₂ | H₂C═CH—CH₂—O |
| A.363 | HC≡C—CH₂ | H₂C═CH—CH₂ |
| A.364 | HC≡C—CH₂ | HC≡C—CH₂—O |
| A.365 | HC≡C—CH₂ | (CH₃)₃C—O |
| A.366 | HC≡C—CH₂ | (CH₃)₂CH—CH₂—O |
| A.367 | HC≡C—CH₂ | (CH₃O)₂C(CH₃) |
| A.368 | HC≡C—CH₂ | (CH₃O)₂CH |
| A.369 | HC≡C—CH₂ | (C₆H₅)—CH₂ |
| A.370 | HC≡C—CH₂ | cyclopropyl |
| A.371 | cyclopropyl | CH₃ |
| A.372 | cyclopropyl | H₃C—CH₂ |
| A.373 | cyclopropyl | H₂C═CH |
| A.374 | cyclopropyl | H₃C—O—CH₂ |
| A.375 | cyclopropyl | ClCH₂—CH₂ |
| A.376 | cyclopropyl | FCH₂—CH₂ |
| A.377 | cyclopropyl | F₃C—CH₂ |
| A.378 | cyclopropyl | Cl₃C—CH₂ |
| A.379 | cyclopropyl | HO—CH₂—CH₂ |
| A.380 | cyclopropyl | H₃C—O—CH₂—CH₂ |
| A.381 | cyclopropyl | (CH₃)₂CH |
| A.382 | cyclopropyl | H₃C—CH₂—CH₂ |
| A.383 | cyclopropyl | CN—CH₂ |
| A.384 | cyclopropyl | CN—CH₂—CH₂ |
| A.385 | cyclopropyl | F₃C—CH₂—CH₂ |
| A.386 | cyclopropyl | Cl₃C—CH₂—CH₂ |
| A.387 | cyclopropyl | H₃C—CH₂—CH₂—CH₂ |
| A.388 | cyclopropyl | H₃C—CH₂—CH(CH₃) |
| A.389 | cyclopropyl | H₃C—CH(CH₃)—CH₂ |
| A.390 | cyclopropyl | H₂C═C(CH₃)—CH₂ |
| A.391 | cyclopropyl | HC≡C—CH(CH₃) |
| A.392 | cyclopropyl | HC≡C—CH₂ |
| A.393 | cyclopropyl | (CH₃)₃C |
| A.394 | cyclopropyl | HO |
| A.395 | cyclopropyl | H₃C—O |
| A.396 | cyclopropyl | H₃C—CH₂—O |
| A.397 | cyclopropyl | (CH₃)₂CH—O |
| A.398 | cyclopropyl | H₃C—CH₂—CH₂—O |
| A.399 | cyclopropyl | H₂C═CH—CH₂—O |
| A.400 | cyclopropyl | H₂C═CH—CH₂ |
| A.401 | cyclopropyl | HC≡C—CH₂—O |
| A.402 | cyclopropyl | (CH₃)₃C—O |
| A.403 | cyclopropyl | (CH₃)₂CH—CH₂—O |
| A.404 | cyclopropyl | (CH₃O)₂C(CH₃) |
| A.405 | cyclopropyl | (CH₃O)₂CH |
| A.406 | cyclopropyl | (C₆H₅)—CH₂ |
| A.407 | cyclopropyl | cyclopropyl |
| A.408 | (C₆H₅)—CH₂ | CH₃ |
| A.409 | (C₆H₅)—CH₂ | H₃C—CH₂ |
| A.410 | (C₆H₅)—CH₂ | H₂C═CH |
| A.411 | (C₆H₅)—CH₂ | H₃C—O—CH₂ |
| A.412 | (C₆H₅)—CH₂ | ClCH₂—CH₂ |
| A.413 | (C₆H₅)—CH₂ | FCH₂—CH₂ |
| A.414 | (C₆H₅)—CH₂ | F₃C—CH₂ |
| A.415 | (C₆H₅)—CH₂ | Cl₃C—CH₂ |
| A.416 | (C₆H₅)—CH₂ | HO—CH₂—CH₂ |
| A.417 | (C₆H₅)—CH₂ | H₃C—O—CH₂—CH₂ |
| A.418 | (C₆H₅)—CH₂ | (CH₃)₂CH |
| A.419 | (C₆H₅)—CH₂ | H₃C—CH₂—CH₂ |
| A.420 | (C₆H₅)—CH₂ | CN—CH₂ |
| A.421 | (C₆H₅)—CH₂ | CN—CH₂—CH₂ |
| A.422 | (C₆H₅)—CH₂ | F₃C—CH₂—CH₂ |
| A.423 | (C₆H₅)—CH₂ | Cl₃C—CH₂—CH₂ |
| A.424 | (C₆H₅)—CH₂ | H₃C—CH₂—CH₂—CH₂ |
| A.425 | (C₆H₅)—CH₂ | H₃C—CH₂—CH(CH₃) |
| A.426 | (C₆H₅)—CH₂ | H₃C—CH(CH₃)—CH₂ |
| A.427 | (C₆H₅)—CH₂ | H₂C═C(CH₃)—CH₂ |
| A.428 | (C₆H₅)—CH₂ | HC≡C—CH(CH₃) |
| A.429 | (C₆H₅)—CH₂ | HC≡C—CH₂ |
| A.430 | (C₆H₅)—CH₂ | (CH₃)₃C |
| A.431 | (C₆H₅)—CH₂ | HO |
| A.432 | (C₆H₅)—CH₂ | H₃C—O |
| A.433 | (C₆H₅)—CH₂ | H₃C—CH₂—O |
| A.434 | (C₆H₅)—CH₂ | (CH₃)₂CH—O |
| A.435 | (C₆H₅)—CH₂ | H₃C—CH₂—CH₂—O |
| A.436 | (C₆H₅)—CH₂ | H₂C═CH—CH₂—O |
| A.437 | (C₆H₅)—CH₂ | H₂C═CH—CH₂ |
| A.438 | (C₆H₅)—CH₂ | HC≡C—CH₂—O |
| A.439 | (C₆H₅)—CH₂ | (CH₃)₃C—O |
| A.440 | (C₆H₅)—CH₂ | (CH₃)₂CH—CH₂—O |
| A.441 | (C₆H₅)—CH₂ | (CH₃O)₂C(CH₃) |
| A.442 | (C₆H₅)—CH₂ | (CH₃O)₂CH |
| A.443 | (C₆H₅)—CH₂ | (C₆H₅)—CH₂ |
| A.444 | (C₆H₅)—CH₂ | cyclopropyl |

The invention also relates to new 3-amino-1,2-benzisothiazole compounds of the follwing formula I

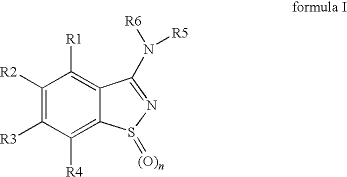

wherein
n is 0, 1 or 2;
R¹ is nitro, cyano, azido, amino, halogen, sulfonylamino, sulfenylamino, sulfinylamino, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, C₁-C₆-alkylthio, (C₁-C₆-alkyl)amino, di(C₁-C₆-alkyl)amino, C₁-C₆-alkylsulfinyl or C₁-C₆-alkylsulfonyl, wherein the eleven last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy, and wherein
   R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl, aryl-C₁-C₆-alkyl, 3- to 7-membered heteroaryl or heteroaryl-C₁-C₆-alkyl, wherein the heteroaryl ring contains as ring members 1, 2 or 3 heteroatoms, selected from the group consisting of nitrogen, oxygen, sulfur, a group SO, SO₂ and N—Rⁿ, wherein Rⁿ is hydrogen, C₁-C₆-alkyl or (C₁-C₆-alkyl-)carbonyl;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, (C₁-C₄-alkoxy)carbonyl, amino, (C₁-C₄-alkyl)amino, di(C₁-C₄-alkyl)amino, aminocarbonyl, (C₁-C₄-alkyl)aminocarbonyl, di(C₁-C₄-alkyl)aminocarbonyl, sulfonyl, sulfonylamino, sulfenylamino, sulfanylamino and C(═O)—R^{2a} or C(═O)—R^{3a} or C(═O)—R^{4a}, and wherein, R^{2a} or R^{3a} or R^{4a} are selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl or heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ and N—Rⁿ, wherein Rⁿ is hydrogen, C₁-C₆-alkyl or (C₁-C₆-alkyl)-carbonyl;
R⁵ is selected from the group consisting of OR^{5a}, C₁-C₁₀-alkyl, C₂-C₁₀-alkenyl, C₂-C₁₀-alkinyl, C₃-C₁₀-cycloalkyl, wherein the four last-mentioned radicals may be unsubstituted, partially or fully halogenated and/or may carry 1-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy-)carbonyl, cyano, nitro, amino, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry substituents selected from the group consisting of C₁-C₆-alkyl, C₁-C₆-haloalkyl, C₁-C₆-alkoxy, C₁-C₆-haloalkoxy and wherein
   R^{5a} is selected from hydrogen, C₁-C₁₀-alkyl, C₁-C₁₀-acyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl and wherein the carbon atoms of the radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
R⁶ is selected from the group consisting of C₁-C₁₀-alkyl, C₂-C₁₀-alkenyl, C₂-C₁₀-alkinyl, C₃-C₁₀-cycloalkyl, wherein these radicals may be unsubstituted, partially or fully halogenated and/or may carry 1-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy-)carbonyl, cyano, nitro, amino, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry substituents selected from the group consisting of C₁-C₆-alkyl, C₁-C₆-haloalkyl, C₁-C₆-alkoxy or C₁-C₆-haloalkoxy;
   and wherein the compound of formula I does not represent 4-chloro-3-N,N-amino-dimethyl-1,2 benzisothiazole.

Preferred are 3-amino-1,2-benzisothiazole compounds of formula I or compositions comprising them, wherein n is 2.

Preferred are also 3-amino-1,2-benzisothiazole compounds of formula I or compositions comprising them, wherein n is 0.

In particular preferred are 3-amino-1,2-benzisothiazole compounds of formula I or compositions comprising them, wherein
R¹ is halogen, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy, C₁-C₄-haloalkoxy, (C₁-C₄-alkoxy)carbonyl and C₃-C₈-cycloalkyl and wherein
   R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl, aryl-C₁-C₄-alkyl.

Especially preferred are those 3-amino-1,2-benzisothiazol compounds of formula I or compositions comprising them, wherein
R¹ is halogen, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy and C₁-C₄-haloalkoxy.

3-Amino-1,2-benzisothiazole derivatives of the present invention can be prepared by different methods of preparations using different precursors.

### P.1. Chloro-benzoisothiazoles

3-Chloro-benzo[d]isothiazole 1,1-dioxides (P-I) can be prepared by the reaction of a suitably substituted saccharine (P-II) with a chlorinating agent such as CICO₂CCl₃, PCl₅/POCl₃ or SOCl₂ as described by D. Dopp et al. in Synthesis 2001, 8, 1228-1235, by R. Salman in Chem. Eng. Data 1987, 32, 391 or by R. W. Lang in Helvetica Chimica Acta 1989, 72, 1248-1252.

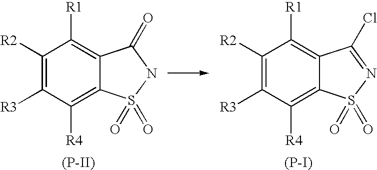

Substituted saccharines (P-II) can be prepared via reaction of 2-chlorosulfonyl-benzoic acid esters (P-III) with ammonia as it is described by M. C. Bell et al. in Bioorganic & Medicinal Letters 1991, No. 12, 733-736 or M. L. Trudell et al. in Journal of Heterocyclic Chem. 2004, 41, 435.

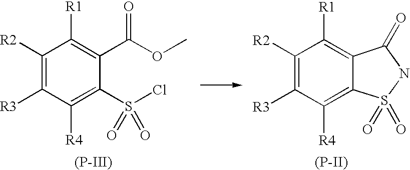

The latter article also describes the synthesis of mentioned 2-chlorosulfonyl-benzoic acid esters (P-III) from the corresponding methyl anthranilates (P-IV) via diazotation and subsequent chlorosulfonation. A similar synthetic procedure is described by G. Hamprecht et al. in Chimia (2004), 58 117-122.

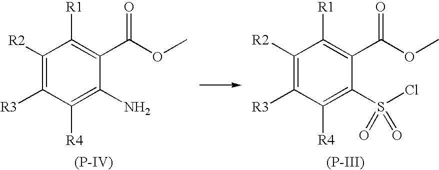

In cases where the methyl anthranilates are not commercially available, they can be prepared from the corresponding 2-nitro benzoic acid methyl ester (P-V) via catalytic hydrogenation as mentioned by J. F. W. Keana et al. in Bioorganic & Medicinal Chemistry 11 (2003) 1769-1780.

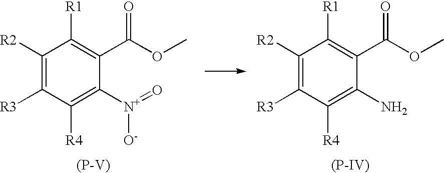

Alternatively, saccharines (P-II) can be prepared by cleavage of the corresponding N-t-butyl saccharines (P-VI) via heating with a strong acid such as trifluoroacetic acid in a way described by K. F. Burri in Helvetica Chimica Acta 1990, 73, 69-80.

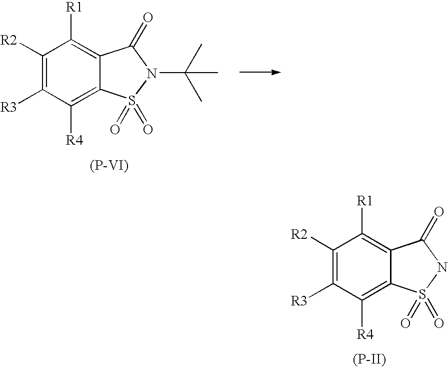

N-tButyl saccharines (P-VI) can be obtained from the corresponding sulfonamides (P-Via) by directed ortho metallation with bases such as butyllithium or lithiumdiisopropylamide and subsequent trapping of the metallated species with carbon dioxide under ring-closure. The procedure is described by D. Becker et al. in Tetrahedron 1992, 2515-2522. The metallation can be carried out as described by N. Murugesan et al. in J. Med. Chem. 1998, 41, 5198-5218.

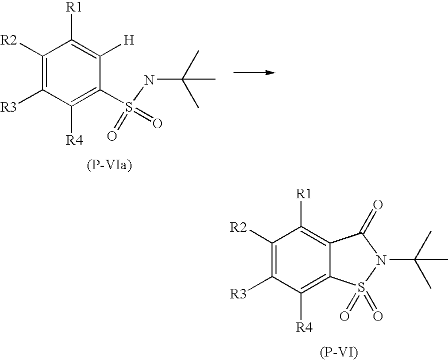

### P.2. Aminobenzisothiazole:

Aminobenzisothiazoles (P-VII) can be prepared by heating a suitably substituted disulfide (P-VIII) together with an amine and an oxidizing agent such as dimethylsulfoxide (DMSO) in a polar solvent such as isopropanol as described by S. W. Walinsky et al. in Organic Process Research & Development 1999, 3, 126-130. Alternatively, the addition can be carried out by using said amines in combination with Grignard-reagents such as n-propyl magnesium chloride and copper(II) salts as oxidizing agents in a solvent such as tetrahydrofurane (THF) as described by T. Nakamura et al. in Synthesis 1997, 871-873.

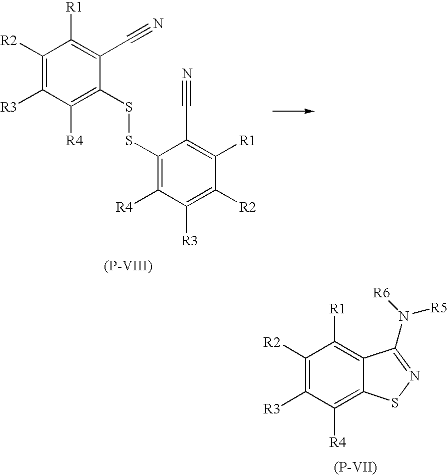

The 2-cyano-disulfides (P-VIII) can be be prepared from the corresponding thiophenols (P-IX) by using oxidizing agents such as dimethylsulfoxid (DMSO) as reported by H. Boerzel et al. in Inorganic Chemistry 2003, 1604-1615.

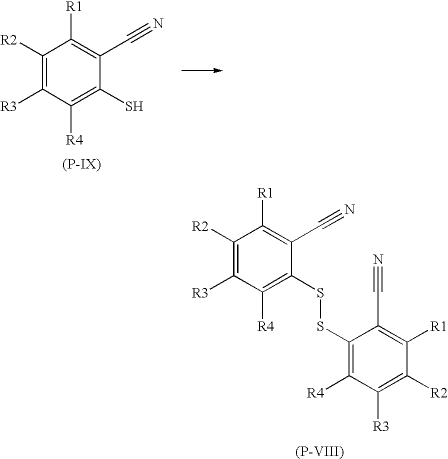

2-cyano-thiophenols (P-IX) can be prepared from benzisothiazoles (P-X) by treatment with strong bases such as NaOCH₃ as described by J. Markert et al. in Liebigs Annalen d. Chemie 1980, 768-778.

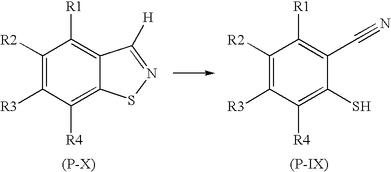

Said article also describes the synthesis of substituted benzisothiazoles (P-X) from 2-chloro-benzaldahydes (P-XI) via reaction with sulfur and ammonia.

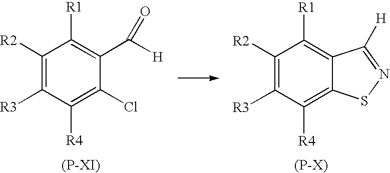

Alternatively, the 2-cyano-disulfides (P-VIII) can be prepared from 2-cyano-anilines (P-XII) via diazotation and subsequent quenching of the diazonium-salt with Na₂S and sulfur as described by V. M. Negrimovsky et al. in Phosphorus, Sulfur & The Related Elements 1995, 104, 161-167.

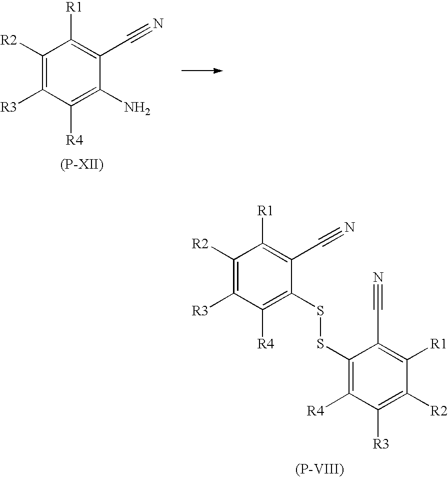

2-cyano-anilines (P-XII) can be prepared from 2-cyano-nitrobenzenes (P-XIII) with a reducing agent such as iron as described by D. H. Klaubert in J. Med. Chem. 1981, 24, 742-748.

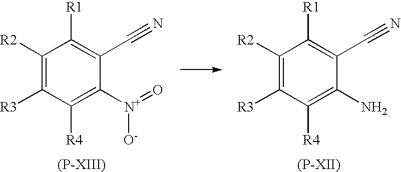

3-amino-1,2-benzisothiazoles (P-VII) can also be prepared from 3-chloro-benzo[d]isothiazole (P-XIIIa) as described by H. Boeshagen et al. in Justus Liebig Annalen der Chemie, 1977, 20 or from trifluoro-methanesulfonic acid benzo[d]isothiazol-3-yl ester (P-XIIIb) in analogy to U.S. Pat. No. 5,359,068 by reaction of said compounds with amines.

3-Chloro-benzo[d]isothiazoles (P-XIIIa) can be obtained from benzisothiazolones (P-XIV) by reaction with a chlorinating agent such as PCl₃ (described by J. P. Yevich et al. in Journal of Medicinal Chemistry 1986, 29, 359-369) or PCl₃/PCl₅ (S. G. Zlutin et al. Journal of Organic Chemistry 2000, 65, 8439-8443).

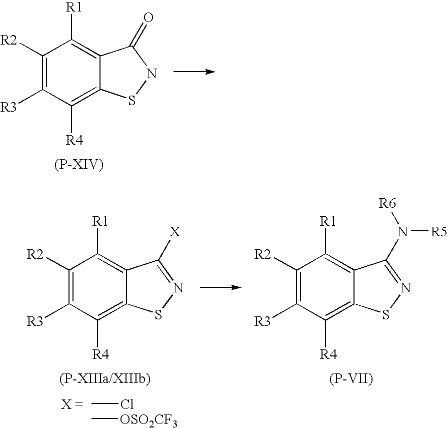

The last mentioned reference also describes the synthesis of said benzisothiazolones from compounds (P-XV) via treatment with an oxidizing agent such as SO₂Cl₂.

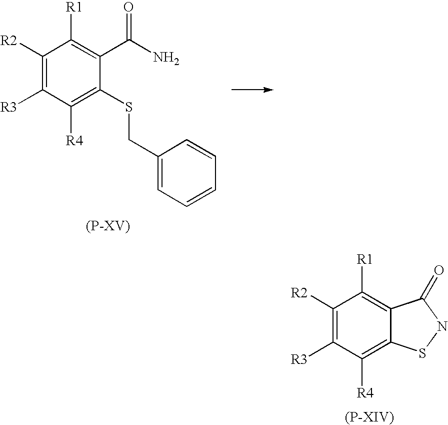

Alternatively, another method for preparing benzisothiazolones (P-XIV) from 2-(alkylhio)benzonitriles (P-XVI) in a one-pot procedure is described in EP 1081141.

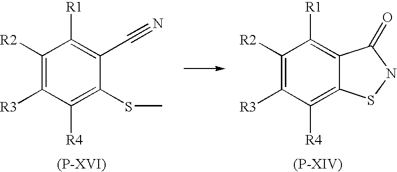

The reaction mixtures are worked up in a customary manner, for example by mixing with water, phase separation and, if appropriate, chromatographic purification of the crude products. In some cases, the intermediates and end products are obtained in the form of colorless or pale brown viscous oils, which are purified or freed from volatile components under reduced pressure and at moderately elevated temperature. If the intermediates and end products are obtained as solids, they can also be purified by recrystallization or digestion.

If individual compounds I are not obtainable by the route described above, they can be prepared by derivatization of other compounds I or by customary modifications of the synthesis routes described.

The preparation of the compounds of formula I may lead to them being obtained as isomer mixtures. If desired, these can be resolved by the methods customary for this purpose, such as crystallization or chromatography, also on optically active adsorbate, to give the pure isomers.

Agronomically acceptable salts of the compounds I can be formed in a customary manner, e.g. by reaction with an acid of the anion in question.

### Pests

The compounds of the formula I are suitable for efficiently controlling nematodes, insects and arachnids in crop protection. In particular, they are suitable for controlling the following animal pests:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

Beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

Dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,

Thrips (Thysanoptera), e.g. Frankliniella fusca, Frankliniella occidentalis, Frankliniella tritici, Scirtothrips citri, Thrips oryzae, Thrips palmi and Thrips tabaci,

Hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta,

Heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,

Homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii.

Termites (Isoptera), e.g. Calotermes flavicollis, Leucotermes flavipes, Reticulitermes lucifugus and Termes natalensis,

Orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus,

Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;

Nematodes, including plant parasitic nematodes and nematodes living in the soil. Plant parasitic nematodes include, such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species;

Dagger nematodes, Xiphinema species; and other plant parasitic nematode species.

The compounds 1, and compositions containing them, are especially useful for the control of insects and nematodes.

Moreover, the compounds 1, and compositions containing them, are especially useful for the control of pests selected from the orders Homoptera, Lepidoptera, Diptera, Thysanoptera, and Nematoda.

In a preferred embodiment of the invention the compounds of formula I are used for controlling insects or arachnids, in particular insects of the orders Lepidoptera, Coleoptera and Homoptera and arachnids of the order Acarina. The compounds of the formula I according to the present invention are particularly useful for controlling insects of the order Lepidoptera and Homoptera.

### Formulations

For use in a method according to the present invention, the compounds I can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula I according to the present invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones (N-metyhl-pyrrolidone (NMP), N-octylpyrrolidone NOP), acetates (glycol diacetate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters,

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example dichlorophen und benzyl alcohol hemiformal

Suitable thickeners are compounds, which confer a pseudoplastic flow behavior to the formulation, i.e. high viscosity at rest and low viscosity in the agitated stage. Mention may be made, in this context, for example, of commercial thickeners based on polysaccharides, such as Xanthan Gum® (Kelzan® from Kelco), Rhodopol®23 (Rhone Poulenc) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compound of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1% per weight.

The active ingredients may also be used successfully in the ultra-low-volume process (ULV), it being possible to apply formulations comprising over 95% by weight of active ingredient, or even to apply the active ingredient without additives.

The following are examples of formulations:

1. Products for dilution with water. For seed treatment purposes, such products may be applied to the seed diluted or undiluted.

### A) Water-Soluble Concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active compound is obtained.

### B) Dispersible Concentrates (DC)

20 parts by weight of the active compound is dissolved in 70 parts by weight of cyclohexanone with addition of 10 parts by weight of a dispersant, for example polyvinylpyrrolidone. Dilution with water gives a dispersion, whereby a formulation with 20% (w/w) of active compounds is obtained.

### C) Emulsifiable Concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

### D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

### E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

### F) Water-Dispersible Granules and Water-Soluble Granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

### G) Water-Dispersible Powders and Water-Soluble Powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

### H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.

2. Products to be applied undiluted for foliar applications. For seed treatment purposes, such products may be applied to the seed diluted or undiluted.

### I) Dustable Powders (DP, DS)

5 parts by weight of the active compound are ground finely and mixed intimately with 95 parts by weight of finely divided kaolin. This gives a dustable product having 5% (w/w) of active compound.

### J) Granules (GR, FG, GG, MG)

0.5 part by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

### K) ULV Solutions (UL)

10 parts by weight of the active compound is dissolved in 90 parts by weight of an organic solvent, for example xylene. This gives a product having 10% (w/w) of active compound, which is applied undiluted for foliar use.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds and compositions of the present invention compounds I may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These additional agents can be admixed with the agents used according to the invention in a weight ratio of 1:10 to 10:1. Mixing the compounds I or the compositions comprising them in the use form as pesticides with other pesticides frequently results in a broader pesticidal spectrum of action.

The following list M of pesticides together with which the compounds according to the invention can be used and with which potential synergistic effects might be produced, is intended to illustrate the possible combinations, but not to impose any limitation:

M.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
M.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
M.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, empenthrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
M.4. Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
M.5. Nicotinic receptor agonists/antagonists compounds: clothianidin, dinotefuran, imidacloprid, thiamethoxam, nitenpyram, acetamiprid, thiacloprid and AKD-1022;
M.6. GABA antagonist compounds: acetoprole, endosulfan, ethiprole, fipronil, vaniliprole, pyrafluprole, pyriprole, the phenylpyrazole compound of formula Γ²

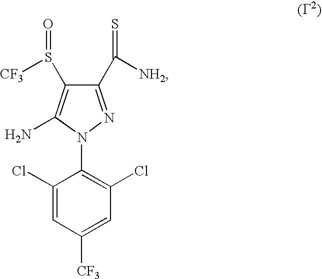

M.7. Macrocyclic lactone insecticides: abamectin, emamectin, milbemectin, lepimectin, spinosad
M.8. METI I compounds: fenazaquin, pyridaben, tebufenpyrad, tolfenpyrad, flufenerim;
M.9. METI II and III compounds: acequinocyl, fluacyprim, hydramethylnon;
M.10. Uncoupler compounds: chlorfenapyr;
M.11. Oxidative phosphorylation inhibitor compounds: cyhexatin, diafenthiuron, fenbutatin oxide, propargite;
M.12. Moulting disruptor compounds: cyromazine;
M.13. Mixed Function Oxidase inhibitor compounds: piperonyl butoxide;
M.14. Sodium channel blocker compounds: indoxacarb, metaflumizone,
M.15. Various: amitraz, benclothiaz, bifenazate, cartap, flonicamid, pyridalyl, pymetrozine, sulfur, thiocyclam, flubendiamide, cyenopyrafen, flupyrazofos, cyflumetofen, amidoflumet, pyrifluquinazon, the aminoquinazolinone compound of formula Γ⁴

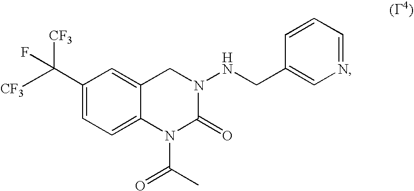

N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, anthranilamide compounds as chlorantraniliprole or the compound of formula Γ⁵

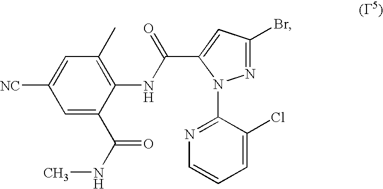

and malononitrile compounds as described in JP 2002 284608, WO 02/89579, WO 02/90320, WO 02/90321, WO 04/06677, WO 04/20399, JP 2004 99597, WO 05/68423, WO 05/68432, or WO 05/63694, especially the malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, and CF₃CF₂CH₂C(CN)₂CH₂(CF₂)₃CF₂H.

The commercially available compounds of the group M may be found in The Pesticide Manual, 13^{th} Edition, British Crop Protection Council (2003) among other publications.

Thioamides of formula Γ² and their preparation have been described in WO 98/28279. Lepimectin is known from Agro Project, PJB Publications Ltd, Nov. 2004. Benclothiaz and its preparation have been described in EP-A1 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180. The aminoquinazolinone compound of formula Γ⁴ has been described in EP A 109 7932. Anthranilamides as the one of formula Γ⁵ or as chloranthraniliprole and their preparations have been described in WO 01/70671; WO 02/48137; WO 03/24222, WO 03/15518, WO 04/67528; WO 04/33468; and WO 05/118552. The malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, and CF₃CF₂CH₂C(CN)₂CH₂(CF₂)₃CF₂H have been described in WO 05/63694.

Fungicidal mixing partners are those selected from the group F consisting of

F.1 acylalanines such as benalaxyl, metalaxyl, ofurace, oxadixyl;
F.2 amine derivatives such as aldimorph, dodine, dodemorph, fenpropimorph, fenpropidin, guazatine, iminoctadine, spiroxamin, tridemorph;
F.3 anilinopyrimidines such as pyrimethanil, mepanipyrim or cyrodinyl;
F.4 antibiotics such as cycloheximid, griseofulvin, kasugamycin, natamycin, polyoxin or streptomycin;
F.5 azoles such as bitertanol, bromoconazole, cyproconazole, difenoconazole, dinitroconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol;
F.6 dicarboximides such as iprodion, myclozolin, procymidon, vinclozolin;
F.7 dithiocarbamates such as ferbam, nabam, maneb, mancozeb, metam, metiram, propineb, polycarbamate, thiram, ziram, zineb;
F.8 heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine;
F.9 copper fungicides such as Bordeaux mixture, copper acetate, copper oxychloride, basic copper sulfate;
F.10 nitrophenyl derivatives such as binapacryl, dinocap, dinobuton, nitrophthalisopropyl;
F.11 phenylpyrroles such as fenpiclonil or fludioxonil;
F.12 strobilurins such as azoxystrobin, dimoxystrobin, fluoxastrobin, kresoxim-methyl, metominostrobin, orysastrobin, picoxystrobin or trifloxystrobin;
F.13 sulfenic acid derivatives such as captafol, captan, dichlofluanid, folpet, tolylfluanid;
F.14 cinnemamides and analogs such as dimethomorph, flumetover or flumorph;
F.15 sulfur, and other fungicides such as acibenzolar-5-methyl, benthiavalicarb, carpropamid, chlorothalonil, cyflufenamid, cymoxanil, dazomet, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid.

### Applications

The animal pest, i.e. the insects, arachnids and nematodes, the plant, soil or water in which the plant is growing can be contacted with the present compound(s) I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

Moreover, animal pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula I are effective through both contact (via soil, glass, wall, bed net, carpet, plant parts or animal parts), and ingestion (bait, or plant part).

For use against ants, termites, wasps, flies, mosquitos, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

For use in treating crop plants, the rate of application of the active ingredients of this invention may be in the range of 0.1 g to 4000 g per hectare, desirably from 25 g to 600 g per hectare, more desirably from 50 g to 500 g per hectare.

### Seed Treatment

The compounds of formula I are also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably aa method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

| | |
|---|---|
| A | Soluble concentrates (SL, LS) |
| D | Emulsions (EW, EO, ES) |
| E | Suspensions (SC, OD, FS) |
| F | Water-dispersible granules and water-soluble granules (WG, SG) |
| G | Water-dispersible powders and water-soluble powders (WP, SP, WS) |
| H | Gel-Formulations (GF) |
| I | Dustable powders (DP, DS) |

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

### Examples of a Gelling Agent is Carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### SYNTHESIS EXAMPLES

The compounds representing the present invention obtained according to the protocols shown in the synthesis examples below and by customary preparation methods together with their physical data are listed in Table I which follows.

### Synthesis Example 1

### (4-Chloro-1,1-dioxo-1H-1λ⁶-benzo[d]isothiazol-3-yl)-isobutyl-methyl-amine (Compound N^{o} 22 of Table C)

### Intermediate Example 1.1: 3,4-Dichloro-benzo[d]isothiazole 1,1-dioxide

6.53 g (30.0 mmol) 4-Chlorosaccharine, 5.4 g (45.0 mmol) thionyl chloride and 0.3 ml dimethylformamid (DMF) were added to 30 ml 1,4-dioxane and heated to reflux for 48 h. The mixture was cooled to room temperature and all volatiles were removed by distillation. 5.3 g (22.5 mmol; 75% of theory) of the title compound were obtained and used in the next step without further purification.

### Compound Example N^{o} 22

### (4-Chloro-1,1-dioxo-1H-1λ⁶-benzo[d]isothiazol-3-yl)-isobutyl-methyl-amine

472 mg (2.0 mmol) 3,4-Dichloro-benzo[d]isothiazole 1,1-dioxide were dissolved in 10 ml dry tetrahydrofuran (THF) and cooled to 0° C. 522 mg (6.0 mmol) N-Methyl-Isobutylamine was dissolved in 10 ml dry tetrahydrofuran (THF) and added slowly. Stirring was continued for 1 h at this temperature and for 14 h at room temperature. The mixture was diluted with dichloromethane and quenched by the addition of saturated aqueous ammonium chloride solution. The layers were separated and the aqueous layer was extracted three times with dichloromethane. The combined organic layers were washed with water, dried over magnesium sulfate and concentrated. Ethyl acetate was added to the residue. The resulting precipitate was collected by filtration, washed with ethyl acetate and dried to afford 264 mg (0.02 mmol; 46% of theory) of the title compound having a melting point of 160-162° C.

### Synthesis Example 2

### Ethyl-(4-methoxy-1,1-dioxo-1H-1λ⁶-benzo[d]isothiazol-3-yl)-methyl-amine (Compound N^{o} 30 of Table C)

### Intermediate Compound 2.1: 3-Methoxy-benzenesulfonyl chloride

50.00 g (0.406 mol) 3-Methoxy-aniline were added to 120 ml acetic acic and cooled to 5-10° C. 30.81 g NaNO₂ were dissolved in 50 ml water and added over 30 min at this temperature.

In a second flask, SO₂ was bubbled through 230 ml acetic acid until saturation. 20.20 g (0.150 mol) CuCl₂ was added and the content of the first flask was added slowly maintaining the reaction temperature in the range of 0-10° C. Stirring was continued until the gas-evolution subsided. The mixture was extracted three times with dichloromethane. The combined organic layers were washed with water, dried over magnesium sulfate and concentrated to afford 66 g of the title product as a crude product which was used in the next step without further purification.

### Intermediate Compound 2.2: N-tert-Butyl-3-methoxy-sulfonamide

5.80 g (28.1 mmol) 3-Methoxy-benzenesulfonyl chloride were dissolved in 100 ml tetrahydrofuran (THF). 2.93 g (28.1 mmol) t-butyl amine were added, followed by a solution of 5.89 g NaHCO₃ in 20 ml of water. Stirring was continued for 14 h at room temperature. The mixture was concentrated and the remaining mixture was extracted three times with ethyl acetate. The combined organic layers were washed with water, dried over magnesium sulfate and concentrated to afford 6.8 g of the title product which was used in the next step without further purification.

¹H-NMR (CDCl₃): δ [ppm]=1.22 (s, 9H), 3.86 (s, 3H), 5.18 (s, 1H), 7.07 (d 1H), 7.38 (t, 1H), 7.45 (s, 1H), 7.51 (d, 1H).

### Intermediate Compound 2.3: N-tert-Butyl-4-methoxy-saccharine

20.0 g (82.2 mmol) N-tert-Butyl-3-methoxy-sulfonamide were dissolved in 200 ml dry tetrahydrofuran (THF) under inert atmosphere and cooled to 0° C. 130 ml (208 mmol) of a 1.6 M solution of n-butyl lithium in hexanes was added slowly. Stirring was continued for 1 h at this temperature before the mixture was cooled to (−78)° C. 15.5 g (164 mmol) methylchloroformate was added and stirring was continued for 2 h at this temperature. Upon warming to room temperature, the mixture was diluted with methyl-t-butyl ether and quenched by the addition of saturated aqueous ammonium chloride solution. The layers were separated, the organic layer was washed with brine, dried over magnesium sulfate and concentrated. The residue was purified on silica (cyclohexane/ethyl acetate 6:1) to afford 14.0 g (52.0 mmol; 63% of theory) of the title compound.

¹H-NMR (CDCl₃): δ [ppm]=1.78 (s, 9H), 4.04 (s, 3H), 7.22 (d, 1H), 7.39 (d 1H), 7.76 (t, 1H)

### Intermediate Compound 2.4: 4-Methoxy-saccharine

5.60 g (20.8 mmol) N-tert-Butyl-4-methoxy-saccharine were dissolved in 20 g trifluoroacetic acid and heated to reflux for 3 h. All volatiles were removed by distillation. Water was added to the residue. The aqueous layer was extracted three times with ethyl acetate. The combined organic layers were dried over magnesium sulfate and concentrated. Dichloromethane was added until the formation of a precipitate started. The precipitate was collected by filtration and dried to afford 2.65 g of product which was used in the next step without further purification.

¹H-NMR (CDCl₃): δ [ppm]=4.07 (s, 3H), 7.27 (d, 1H), 7.46 (d, 1H), 7.83 (t, 1H).

### Intermediate Compound 2.5: 3-Chloro-4-methoxy-benzo[d]isothiazole 1,1-dioxide

1.80 g (8.44 mmol) 4-Methoxy-saccharine, 1.51 g (12.66 mmol) thionyl chloride and 0.15 g dimethylformamid (DMF) were added to 20 ml 1,4-dioxane and heated to reflux for 30 h. The mixture was cooled to room temperature and all volatiles were removed by distillation. 2.06 g of the title compound were obtained as a crude product and used in the next step without further purification.

### Compound Example N^{o} 30

### Ethyl-(4-methoxy-1,1-dioxo-1H-1-λ⁶-benzo[d]isothiazol-3-yl)-methyl-amine

500 mg (2.16 mmol) 3-Chloro-4-methoxy-benzo[d]isothiazole 1,1-dioxide were dissolved in 5 ml dry tetrahydrofuran (THF) and slowly added to a solution of 380 mg (6.47 mmol) ethyl-methyl-amine at 0° C. Stirring was continued for 1 h at this temperature and for 14 h at room temperature. The mixture was diluted with dichloromethane and quenched by the addition of saturated aqueous ammonium chloride solution. The layers were separated and the aqueous layer was extracted two times with dichloromethane. The combined organic layers were dried over magnesium sulfate and concentrated. Cyclohexane/ethyl acetate 2:1 was added until the formation of a precipitate started. The precipitate was collected by filtration and dried to afford 209 mg (0.82 mmol; 38% of theory) of the title compound having a melting point of 154-156° C.

### Synthesis Example 3

### (4-Difluoromethoxy-1,1-dioxo-1H-1λ⁶-benzo[d]isothiazol-3-yl)-diethyl-amine (Compound N^{o} 31 of Table C)

### Intermediate Compound 3.1: N-tert-Butyl-4-hydroxy-saccharine

5.00 g (18.6 mmol) N-tert-Butyl-4-methoxy-saccharine were dissolved in 50 ml dichloromethane under inert atmosphere and cooled to (−78)° C. 56.0 ml (56.0 mmol) of a 1M solution of BBr₃ in dichloromethane were added. After completion of the addition completion of the reaction could be observed by Thin Layer Chromatography (TLC). The reaction was quenched by carefully adding water. Upon warming to room temperature, the layers were separated and the organic layer was washed with water, dried over magnesium sulfate and concentrated to afford 3.50 g of the title product, which was used in the next step without further purification.

¹H-NMR (CDCl₃): δ [ppm]=1.76 (s, 9H), 7.19 (d, 1H), 7.30 (d, 1H), 7.65 (t, 1H), 9.22 (s, 1H).

### Intermediate Compound 3.2: N-tert-Butyl-4-difluoromethoxy-saccharine

1.00 g (3.92 mmol) N-tert-Butyl-4-hydroxy-saccharine and 1.79 g (12.9 mmol) powdered K₂CO₃ were added to 20 ml dimethylformamid (DMF). The mixture was heated to 110° C. Chlorodifluoromethane was led through the mixture for 5 min. The mixture was concentrated and water and methyl-t-butyl ether were added to the residue. The layers were separated and the organic layer was extracted with methyl-t-butyl ether. The combined organic layer was washed with aqueous 10% NaOH and two times with water, dried over magnesium sulfate and concentrated to afford 1.20 g of the title compound which was used in the next step without further purification.

¹H-NMR (CDCl₃): δ [ppm]=1.78 (s, 9H), 6.80 (t, 1H), 7.55 (d, 1H), 7.71 (d, 1H), 7.82 (t, 1H).

### Intermediate Compound 3.3: 4-Difluoromethoxy-saccharine

10.0 g (32.8 mmol) N-tert-Butyl-4-difluoromethoxy-saccharine were dissolved in 50 ml trifluoroacetic acid and heated to reflux for 30 h. Stirring was continued at room temperature for 64 h. All volatiles were removed by distillation. Water was added to the residue. The aqueous layer was extracted three times with ethyl acetate. The combined organic layers were dried over magnesium sulfate and concentrated to afford 9.5 g of a crude product which was used in the next step without further purification.

¹H-NMR (CDCl₃): δ [ppm]=6.81 (t, 1H), 7.63 (d, 1H), 7.78 (d, 1H), 8.01 (t, 1H).

### Intermediate Compound 3.4: 3-Chloro-4-difluoromethoxy-benzo[d]isothiazole 1,1-dioxide

9.50 g (38.1 mmol) 4-Difluoromethoxy-saccharine, 6.8 g (57.0 mmol) thionyl chloride and 0.4 ml dimethylformamid (DMF) were added to 50 ml 1,4-dioxane and heated to reflux for 29 h. The mixture was cooled to room temperature and all volatiles were removed by distillation. 9.3 g of the title compound were obtained as a crude product and used in the next step without further purification.

### Compound Example N^{o} 31

### (4-Difluoromethoxy-1,1-dioxo-1H-1λ⁶-benzo[d]isothiazol-3-yl)-diethyl-amine

500 mg (1.87 mmol) 3-Chloro-4-difluoromethoxy-benzo[d]isothiazole 1,1-dioxide were dissolved in 15 ml dry tetrahydrofuran (THF) and cooled to 0° C. 340 mg (4.65 mmol) diethyl amine was added slowly. Stirring was continued for 1 h at this temperature and for 48 h at rt. The mixture was diluted with dichloromethane and quenched by the addition of saturated aqueous ammonium chloride solution. The layers were separated and the aqueous layer was extracted three times with dichloromethane. The combined organic layers were washed with water, dried over magnesium sulfate and concentrated. The residue was purified on silica (cyclohexane/ethyl acetate 3:1) to afford 200 mg (0.66 mmol; 35% of theory) of the title compound having a melting point of 93-94° C.

### Synthesis Example 4

### (4-Chloro-benzo[d]isothiazol-3-yl)-dipropyl-amine (Compound N^{o} 43 of Table C)

### Intermediate Compound 4.1: Bis-(3-chloro-2-cyano-phenyl)-disulfide

7.0 g 2-Chloro-6-mercapto-benzonitrile were dissolved in 25 ml dimethyl-sulfoxide and stirred for 6 h at room temperature. A white precipitate was formed and collected by filtration to afford after drying 6.38 g (18.9 mmol; 92% of theory) of the title compound.

¹H-NMR (CDCl₃): δ [ppm]=7.43 (d, 2H), 7.53 (t, 2H), 7.66 (d, 1H).

### Compound Example N^{o} 43

### (4-Chloro-benzo[d]isothiazol-3-yl)-dipropyl-amine

2.00 g (5.93 mmol) bis-(3-chloro-2-cyano-phenyl)-disulfide were added to a solution of 6.00 g (59.3 mmol) di-n-propylamine and 0.51 g (6.52 mmol) dimethylsulfoxide in 2.4 ml isopropanol.

The mixture was heated to reflux for 24 h. The mixture was cooled to rt and all volatiles were removed by distillation. Ethyl acetate was added to the residue. The filtrate was collected by filtration and concentrated. The residue was purified on a C-18 column with acetonitrile/water as the eluent to afford 0.63 g (2.34 mmol) of the title compound as an oily substance.

### Synthesis Example 5

### (5-Bromo-4-methoxy-benzo[d]isothiazol-3-yl)-dimethyl-amine (Compound N^{o} 48 of Table C)

### Intermediate Compound 5.1: 5-Bromo-4-methoxy-1,2-benzisothiazol-3-one

At 10° C. 3.1 g (23 mmol) sulfurylchloride were added to a solution of 5 g (19 mmol) 5-bromo-6-methoxy-2-methylthio-benzonitrile in 90 ml chlorobenzene. The solution was heated to 70° C. and stirred for 2 h. After 12 h at room temperature the precipitate was collected and recrystallized from toluene.

1.9 g (38%) of 5-bromo-4-methoxy-1,2-benzisothiazol-3-one were isolated: melting point 197-198° C.

### Compound Example N^{o} 48

### (5-Bromo-4-methoxy-benzo[d]isothiazol-3-yl)-dimethyl-amine

1.7 g (5.7 mmol) trifluoromethanesulfonic anhydride were added to a solution of 1 g (3.8 mmol) 5-bromo-4-methoxy-1,2-benzisothiazol-3-one and 0.45 g (5.7 mmol) pyridine in 10 ml dichloromethane. The temperature was kept below 35° C. The organic phase was extracted with aqueous 10% HCl, washed with water and dried over NaSO₄.

Yield: 1.31 g (87%), ¹H-NMR (CDCl₃): δ [ppm]=4.05 (s, 3H), 7.50 (d 1H), 7.75 (d, 1H).

To a solution of 0.5 g (1.3 mmol) triflate in 5 ml THF were added 0.17 g (1.5 mmol) dimethylamine and 0.2 g (2 mmol) triethylamine. The solution was stirred for 24 h at room temperature. The mixture was poured into water and extracted with ethyl acetate. The organic phase was washed with potassium carbonate solution and water, dried over NaSO₄ and the solvent was evaporated. The residue was purified on silica (cyclohexane/ethyl acetate 6:1) to afford 90 mg (25%) ¹H-NMR (CDCl₃): δ [ppm]=3.08 (s, 6H), 3.91 (s, 3H), 7.39 (d 1H), 7.60 (d, 1H).

### Table C:

The products were characterized by coupled High Performance Liquid Chromatography/mass spectrometry (HPLC/MS), by NMR or by their melting points. Analytical HPLC column: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C.

Some compounds were characterized by ¹H-NMR. The signals are characterized by chemical shift (ppm) vs. tetramethylsilane, by their multiplicity and by their integral (relative number of hydrogen atoms given). The following abbreviations are used to characterize the multiplicity of the signals: M=multiplett, q=quartett, t=triplett, d=doublet and s=singulett.

Some individual compounds were characterized by their melting point m.p. in ° C.

TABLE C

| | | Physical property |
|---|---|---|
| | | (m.p. [° C.], retention time tᵣ [min], |
| n° of | | mass spectrum m/z (HPLC/MS) or |
| compound | Formula | chemical shift (¹H-NMR)) |
| 1. | 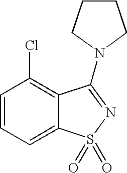 | HPLC-MS:tᵣ = 2.24 minm/z = 271 [M + 1]⁺ |
| 2. | 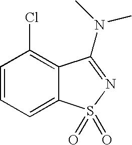 | m.p. = 179-180 |
| 3. | 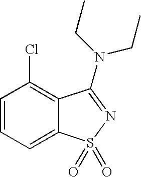 | m.p. = 108-109 |
| 4. | 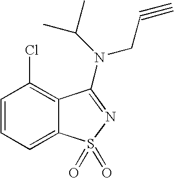 | m.p. = 122-124 |
| 5. | 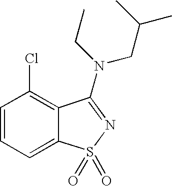 | m.p. = 177-179 |
| 6. | 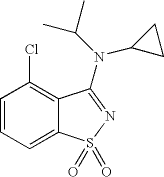 | m.p. = 106-108 |
| 7. | 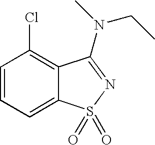 | m.p. = 114-116 |
| 8. | 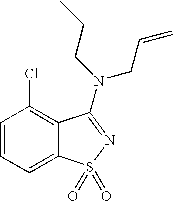 | m.p. = 95-97 |
| 9. | 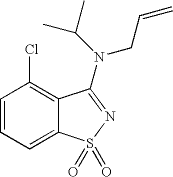 | m.p. = 103-105 |
| 10. | 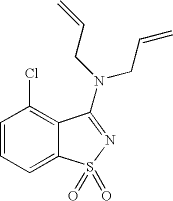 | m.p. = 101-103 |
| 11. | 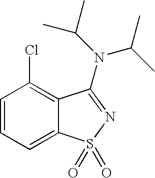 | ¹H-NMR (CDCl₃):1.51 (d, 12 H), 4.20 (m, 2 H), 7.60 (m,2 H), 7.82 (t, 1 H) |
| 12. | 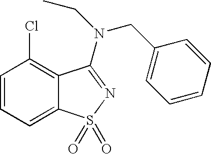 | ¹H-NMR (CDCl₃):1.28 (t, 3 H), 3.58 (q, 2 H), 4.85 (s,2 H), 7.31 (m, 5 H), 7.65 (m, 2 H), 7.79(t, 1 H). |
| 13. | 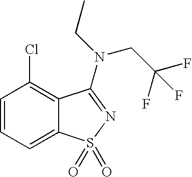 | m.p. = 126-130 |
| 14. | 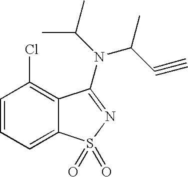 | m.p. = 137-139 |
| 15. | 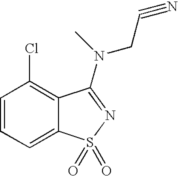 | m.p. = 156-162 |
| 16. | 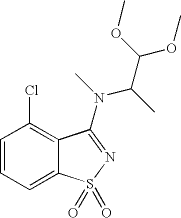 | m.p. = 135-137 |
| 17. | 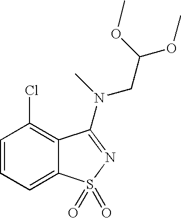 | m.p. = 142-144 |
| 18. | 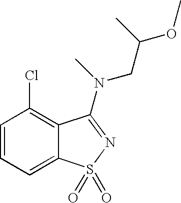 | m.p. = 160-162 |
| 19. | 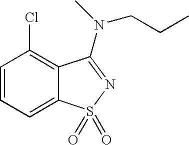 | m.p. = 82-84 |
| 20. | 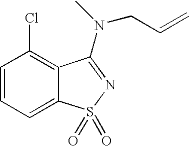 | m.p. = 121-123 |
| 21. | 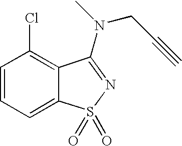 | m.p. = 163-165 |
| 22. | 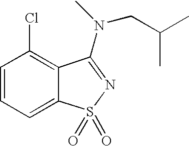 | m.p. = 160-162 |
| 23. | 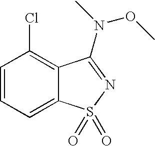 | m.p. = 114-116 |
| 24. | 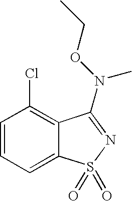 | HPLC-MS:tᵣ = 2.559 minm/z = 275 [M + 1]⁺ |
| 25. | 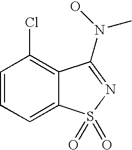 | m.p. = 179-180 |
| 26. | 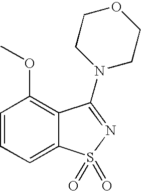 | HPLC-MS:tᵣ = 1.79 minm/z = 283 [M + 1]⁺ |
| 27. | 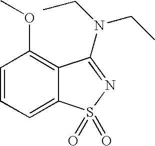 | m.p. = 171-175 |
| 28. | 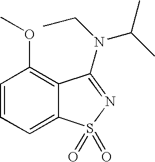 | m.p. = 113-114 |
| 29. | 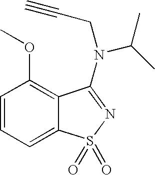 | HPLC-MS:tᵣ = 2.539 minm/z = 293 [M + 1]⁺ |
| 30. | 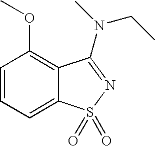 | m.p. = 154-156 |
| 31. | 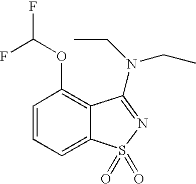 | m.p. = 93-94 |
| 32. | 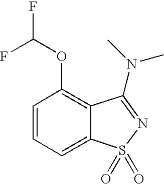 | m.p. = 149-151 |
| 33. | 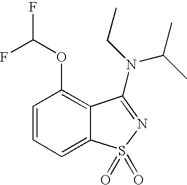 | HPLC-MS:tᵣ = 2.80 minm/z = 319 [M + 1]⁺ |
| 34. | 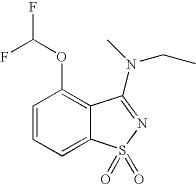 | m.p. = 108-109 |
| 35. | 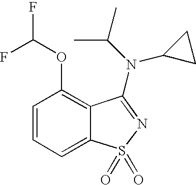 | m.p. = 129-131 |
| 36. | 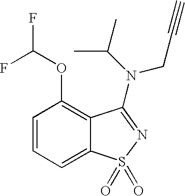 | HPLC-MS:tᵣ = 2.74 minm/z = 329 [M + 1]⁺ |
| 37. | 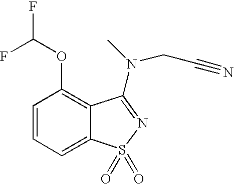 | HPLC-MS:tᵣ = 2.09 minm/z = 302 [M + 1]⁺ |
| 38. | 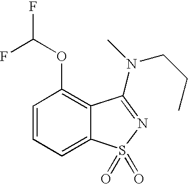 | m.p. = 85-87 |
| 39. | 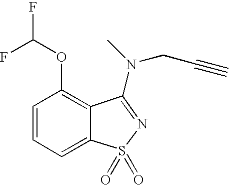 | HPLC-MS:tᵣ = 2.35 minm/z = 301 [M + 1]⁺ |
| 40. | 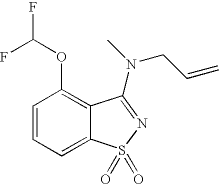 | m.p. = 80-85 |
| 41. | 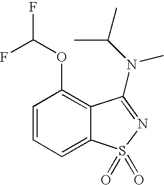 | m.p. = 87-93 |
| 42. | 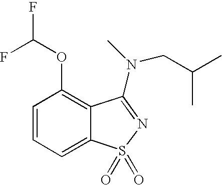 | HPLC-MS:tᵣ = 2.87 minm/z = 319 [M + 1]⁺ |
| 43. | 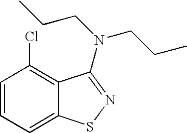 | HPLC-MS:tᵣ = 4.37 minm/z = 269 [M + 1]⁺ |
| 44. | 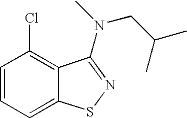 | HPLC-MS:tᵣ = 4.22 minm/z = 255 [M + 1]⁺ |
| 45. | 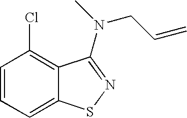 | HPLC-MS:tᵣ = 4.08 minm/z = 239 [M + 1]⁺ |
| 46. | 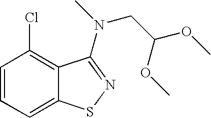 | HPLC-MS:tᵣ = 3.45 minm/z = 287 [M + 1] |
| 47. | 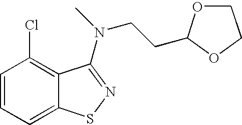 | HPLC-MS:tᵣ = 3.53 minm/z = 299 [M + 1]⁺ |
| 48. | 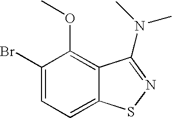 | ¹H-NMR (CDCl₃):3.08 (s, 6 H), 3.91 (s, 3 H), 7.39 (d1 H), 7.60 (d, 1 H) |
| 49. | 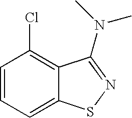 | HPLC-MS:tᵣ = 3.60 minm/z = 213 [M + 1] |
| 50. | 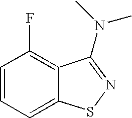 | m.p. = 145-146 |
| 51. | 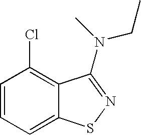 | ¹H-NMR (CDCl₃):1.26 (t, 3 H), 2.96 (s, 3 H), 3.32 (q,2 H), 7.37 (m, 2 H), 7.69 (m, 1 H). |
| 52. | 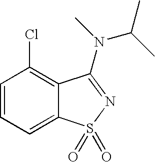 | HPLC-MS:tᵣ = 2.62 minm/z = 273 [M + 1] |
| 53. | 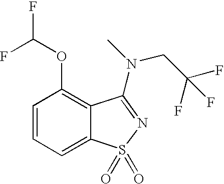 | HPLC-MS:tᵣ = 2.83 minm/z = 345 [M + 1] |
| 54. | 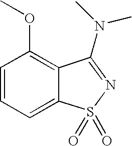 | HPLC-MS:tᵣ = 1.74 minm/z = 241 [M + 1] |
| 55. | 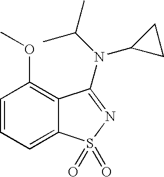 | HPLC-MS:tᵣ = 2.72 minm/z = 295 [M + 1] |
| 56. | 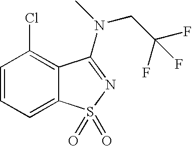 | HPLC-MS:tᵣ = 2.93 minm/z = 313 [M + 1] |
| 57. | 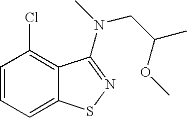 | HPLC-MS:tᵣ = 3.74 minm/z = 271 [M + 1] |
| 58. | 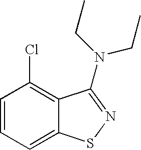 | ¹H-NMR (CDCl₃):1.17 (t, 6 H), 3.39 (q, 4 H), 7.34 (m,2 H), 7.69 (m, 1 H). |
| 59. | 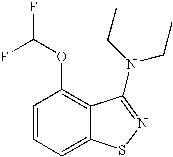 | m.p. = 49-50 |
| 60. | 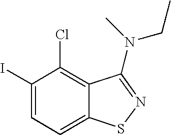 | ¹H-NMR (CDCl₃):1.21 (t, 3 H), 2.94 (s, 3 H), 3.30 (q,2 H), 7.42 (d, 1 H), 7.90 (d, 1 H). |
| 61. | 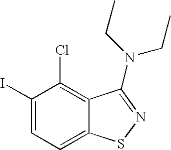 | ¹H-NMR (CDCl₃):1.11 (t, 6 H), 3.34 (q, 4 H), 7.42 (d,1 H), 7.89 (d, 1 H). |
| 62. | 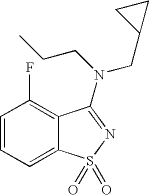 | HPLC-MS:tᵣ = 3.02 minm/z = 297 [M + 1] |
| 63. | 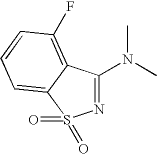 | HPLC-MS:tᵣ = 1.66 minm/z = 229 [M + 1] |
| 64. | 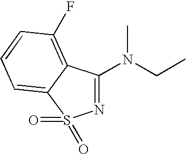 | HPLC-MS:tᵣ = 2.00 minm/z = 243 [M + 1] |
| 65. | 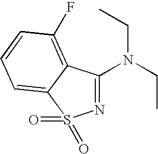 | HPLC-MS:tᵣ = 2.33 minm/z = 257 [M + 1] |
| 66. | 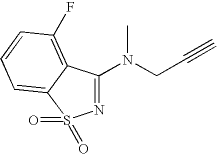 | HPLC-MS:tᵣ = 2.07 minm/z = 253 [M + 1] |
| 67. | 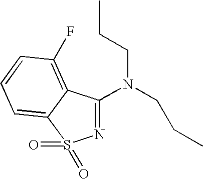 | HPLC-MS:tᵣ = 2.96 minm/z = 285 [M + 1] |
| 68. | 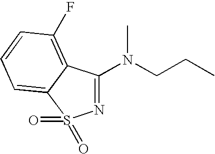 | HPLC-MS:tᵣ = 2.36 minm/z = 257 [M + 1] |
| 69. | 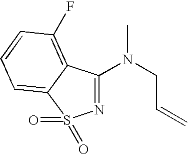 | HPLC-MS:tᵣ = 2.25 minm/z = 255 [M + 1] |
| 70. | 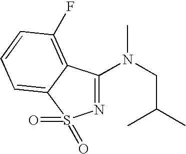 | HPLC-MS:tᵣ = 2.62 minm/z = 271 [M + 1] |
| 71. | 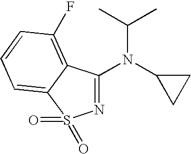 | HPLC-MS:tᵣ = 2.71 minm/z = 283 [M + 1] |
| 72. | 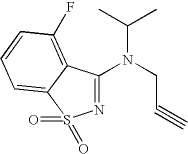 | HPLC-MS:tᵣ = 2.55 minm/z = 281 [M + 1] |
| 73. | 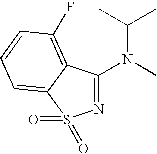 | HPLC-MS:tᵣ = 2.04 minm/z = 257 [M + 1] |
| 74. | 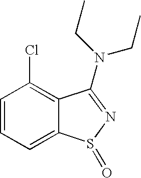 | HPLC-MS:tᵣ = 2.59 minm/z = 257 [M + 1] |
| 75. | 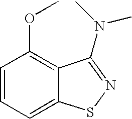 | ¹H-NMR (CDCl₃):3.03 (s, 6 H), 4.02 (s, 3 H), 6.74 (d,1 H), 7.39 (m, 2 H). |
| 76. | 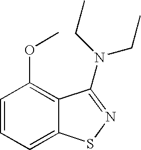 | ¹H-NMR (CDCl₃):1.20 (t, 6 H), 3.42 (q, 4 H), 3.98 (s,3 H), 6.70 (d, 1 H), 7.34 (m, 2 H). |
| 77. | 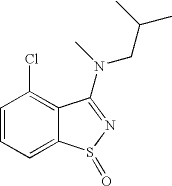 | ¹H-NMR (CDCl₃):0.90 (d, 3 H), 0.97 (d, 3 H), 2.20 (m,1 H), 3.20 (s, 3 H), 3.31 (q, 1 H), 3.62(q, 1 H), 7.60 (m, 2 H), 7.89 (m, 1 H). |
| 78. | 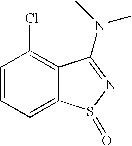 | m.p. = 147-148 |
| 79. | 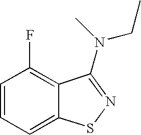 | ¹H-NMR (CDCl₃):1.29 (t, 3 H), 3.02 (s, 3 H), 3.40 (q,2 H), 7.00 (m, 1 H), 7.41 (m, 1 H), 7.55(m, 1 H). |
| 80. | 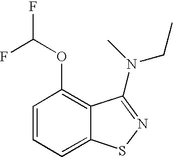 | ¹H-NMR (CDCl₃):1.22 (t, 3 H), 2.99 (s, 3 H), 3.39 (q,2 H), 6.67 (t, 1 H) 7.07 (d, 1 H), 7.46 (t,1 H), 7.65 (d, 1 H). |
| 81. | 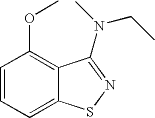 | HPLC-MS:tᵣ = 2.53 minm/z = 223 [M + 1] |
| 82. | 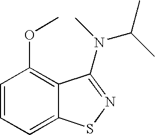 | HPLC-MS:tᵣ = 2.66 minm/z = 237 [M + 1] |
| 83. | 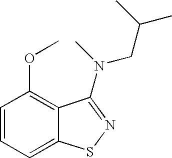 | HPLC-MS:tᵣ = 3.52 minm/z = 251 [M + 1] |
| 84. | 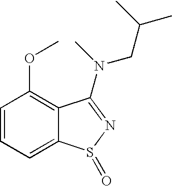 | HPLC-MS:tᵣ = 2.33 minm/z = 267 [M + 1] |
| 85. | 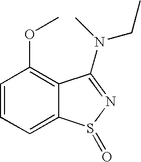 | HPLC-MS:tᵣ = 1.83 minm/z = 239 [M + 1] |
| 86. | 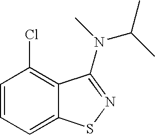 | HPLC-MS:tᵣ = 3.89 minm/z = 241 [M + 1] |
| 87. | 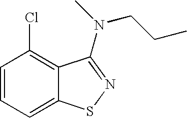 | HPLC-MS:tᵣ = 3.99 minm/z = 241 [M + 1] |
| 88. | 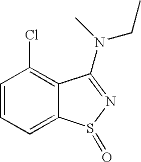 | HPLC-MS:tᵣ = 2.16 minm/z = 243 [M + 1] |
| 89. | 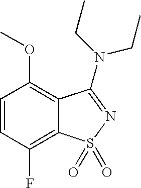 | HPLC-MS:tᵣ = 2.52 minm/z = 287 [M + 1] |
| 90. | 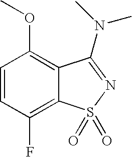 | HPLC-MS:tᵣ = 1.96 minm/z = 259 [M + 1] |
| 91. | 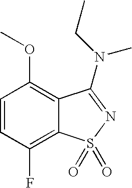 | HPLC-MS:tᵣ = 2.18 minm/z = 273 [M + 1] |
| 92. | 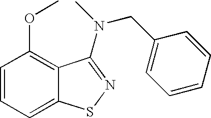 | m.p. = 53-55 |
| 93. | 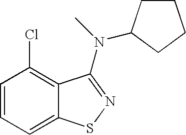 | ¹H-NMR (CDCl₃):1.59 (m, 2 H), 1.70 (m, 4 H), 1.91 (m,2 H), 2.89 (s, 3 H), 4.08 (m, 1 H), 7.35(m, 2 H), 7.69 (m, 1 H). |
| 94. | 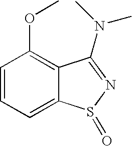 | HPLC-MS:tᵣ = 1.60 minm/z = 225 [M + 1] |
| 95. | 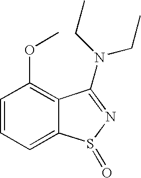 | HPLC-MS:tᵣ = 2.14 minm/z = 253 [M + 1] |
| 96. | 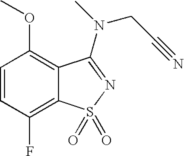 | m.p. = 128-130 |
| 97. | 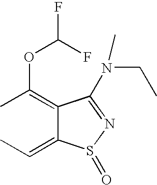 | HPLC-MS:tᵣ = 2.29 minm/z = 275 [M + 1] |
| 98. | 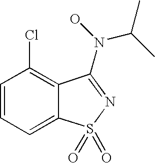 | ¹H-NMR (CDCl₃):1.38 (d, 6 H), 4.70 (m, 1 H), 7.64 (m,2 H), 7.78 (m, 1 H). |
| 99. | 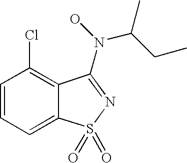 | ¹H-NMR (CDCl₃):0.92 (m, 3 H), 1.40 (d, 3 H), 1.65 (m,1 H), 1.94 (m, 1 H), 4.45 (m, 1 H), 7.65(m, 2 H), 7.78 (m, 1 H). |
| 100. | 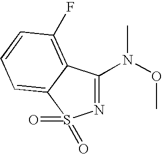 | HPLC-MS:tᵣ = 1.88 minm/z = 245 [M + 1] |
| 101. | 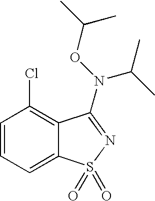 | m.p. = 130-131 |
| 102. | 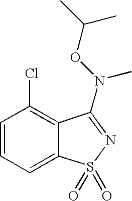 | m.p. = 118-120 |
| 103. | 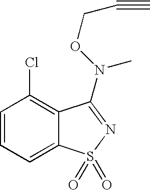 | m.p. = 136-137 |
| 104. | 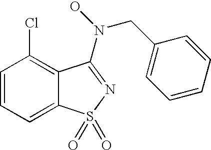 | m.p. = 167-169 |
| 105. | 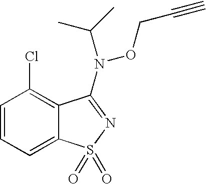 | m.p. = 210-211 |
| 106. | 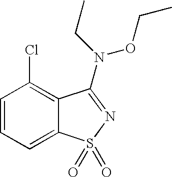 | m.p. = 80-81 |
| 107. | 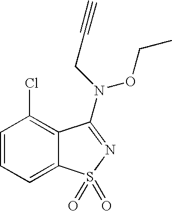 | m.p. = 144-145 |
| 108. | 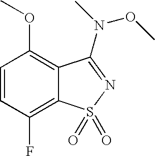 | m.p. = 117-119 |
| 109. | 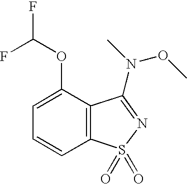 | m.p = 104-105 |
| 110. | 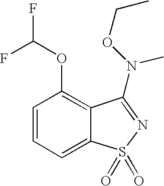 | HPLC-MS:tᵣ = 2.41 minm/z = 307 [M + 1] |
| 111. | 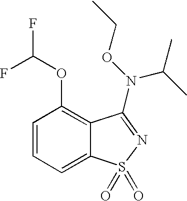 | HPLC-MS:tᵣ = 2.96 minm/z = 335 [M + 1] |
| 112. | 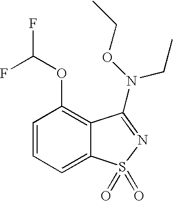 | HPLC-MS:tᵣ = 2.83 minm/z = 321 [M + 1] |

The action of the compounds of the formula I against pests was demonstrated by the following experiments:

B.1 Cotton Aphid (Aphis gossypii)

The active compounds were formulated in 50:50 acetone:water and 100 ppm Kinetic® surfactant.

Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, compounds 1-10, 13-23, 26-42, 44-46, 48, 49, 51-58, 60-74, 76-103 and 104-112 at 300 ppm showed over 86% mortality in comparison with untreated controls.

### B.2 Green Peach Aphid (Myzus Persicae)

The active compounds were formulated in 50:50 acetone:water and 100 ppm Kinetic® surfactant.

Pepper plants in the 2^{nd} leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

In this test, compounds 2, 3, 6, 7, 15, 19, 23, 27, 28, 30-34, 36-42, 48, 49, 51, 53, 57, 58, 62, 64, 73, 74, 76, 78, 80-83, 85-89, 91-98 and 108-112 at 300 ppm showed over 86% mortality in comparison with untreated controls.

B.3. Bean Aphid (Aphis fabae)

The active compounds were formulated in 50:50 acetone:water and 100 ppm Kinetic® surfactant.

Nasturtium plants grown in Metro mix in the 1st leaf-pair stage (variety ‘Mixed Jewel’) were infested with approximately 20-30 laboratory-reared aphids by placing infested cut plants on top of the test plants. The cut plants were removed after 24 hr. Each plant was dipped into the test solution to provide complete coverage of the foliage, stem, protruding seed surface and surrounding cube surface and allowed to dry in the fume hood. The treated plants were kept at about 25° C. with continuous fluorescent light. Aphid mortality is determined after 3 days.

In this test, compounds nos. 3-9, 11, 12, 15, 23, 36 and 49 at 300 ppm showed over 86% mortality in comparison with untreated controls.

1-25. (canceled)

26. A method for combating or controlling insects, arachnids or nematodes comprising contacting an insect, arachnid or nematode or their food supply, habitat or breeding grounds with a pesticidally effective amount of at least a compound of formula I or a composition comprising at least one compound of formula I: 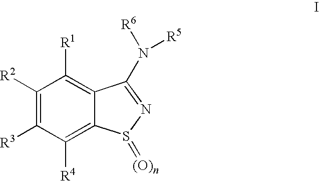
wherein
n is 0, 1 or 2;
R¹ is hydrogen, nitro, cyano, azido, amino, halogen, sulfonylamino, sulfenylamino, sulfinylamino, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, C₁-C₆-alkylthio, (C₁-C₆-alkyl)amino, di(C₁-C₆-alkyl)amino, C₁-C₆-alkylsulfinyl or C₁-C₆-alkylsulfonyl, wherein the eleven last-mentioned radicals may be unsubstituted, partially or fully halogenated and carry 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, wherein the phenyl is unsubstituted, or partially or fully halogenated and carry 0, 1, 2 or 3 substituents, independently of one another selected from the group consisting of C₁-C₄-alkyl, C₁-C₄-haloalkyl, C₁-C₄-alkoxy and C₁-C₄-haloalkoxy, and wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl, aryl-C₁-C₆-alkyl, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂, and N—Rⁿ, wherein Rⁿ is hydrogen, C₁-C₆-alkyl, or (C₁-C₆-alkyl)-carbonyl;
R², R¹ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, (C₁-C₄-alkoxy)carbonyl, amino, (C₁-C₄-alkyl)amino, di(C₁-C₄-alkyl)amino, aminocarbonyl, (C₁-C₄-alkyl)aminocarbonyl, di(C₁-C₄-alkyl)aminocarbonyl, sulfonyl, sulfonylamino, sulfenylamino, sulfanylamino and C(═O)—R^{2a}, C(═O)—R^{3a}, or C(═O)_R^{4a}, wherein each of R^{2a}, R^{3a}, and R^{4a} is selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ or N—Rⁿ, wherein Rⁿ is hydrogen, C₁-C₆-alkyl or (C₁-C₆-alkyl)-carbonyl;
R⁵ is selected from the group consisting of OR^{5a}, C₁-C₆-alkyl, C₂-C₁₀-alkenyl, C₂-C₁₀-alkinyl, and C₃-C₁₀-cycloalkyl, wherein each radical is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy, and C₁-C₆-haloalkoxy, wherein
R^{5a} is selected from the group consisting of hydrogen, C₁-C₁₀-alkyl, C₁-C₁₀-acyl, C₃-C₁₀-cycloalkyl, C₂-C₁₀-alkenyl, C₂-C₁₀-alkynyl, aryl, aryl-C₁-C₄-alkyl, heteroaryl, heteroaryl-C₁-C₄-alkyl, heterocyclyl and heterocyclyl-C₁-C₄-alkyl and wherein one or more carbon atoms of the radicals is unsubstituted, or partially or fully halogenated and carries 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
R⁶ is selected from the group consisting of C₁-C₁₀-alkyl, C₂-C₁₀-alkenyl, C₂-C₁₀-alkinyl, and C₃-C₁₀-cycloalkyl, wherein each of the four last-mentioned radicals is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy and C₁-C₆-haloalkoxy, or
R⁵/R^{5a} and R⁶ can join to form an unsaturated, or partially or fully saturated 3-10 membered ring, unsubstituted or substituted with an optionally substituted C₁-C₅-alkyl, wherein one or more carbon ring atoms are replaced by 0 to 3 heteroatoms selected from the group consisting of N, NRⁿ, O, S, SO, and SO₂ wherein
Rⁿ is hydrogen, C₁-C₆-alkyl or (C₁-C₆-alkyl)-carbonyl;
or the enantiomers or diastereomers, salts or esters thereof.

27. A method for protecting growing plants from attack or infestation by insects, arachnids or nematodes comprising contacting a plant, soil or water in which the plant is growing, with a pesticidally effective amount of at least one compound of formula I or a composition comprising at least one compound of formula I: 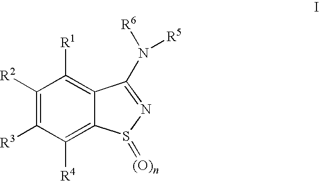
wherein
n is 0, 1 or 2;
R¹ is hydrogen, nitro, cyano, azido, amino, halogen, sulfonylamino, sulfenylamino, sulfinyl amino, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, C₁-C₆-alkylthio, (C₁-C₆-alkyl)amino, di(C₁-C₆-alkyl)amino, C₁-C₆-alkylsulfinyl or C₁-C₆-alkylsulfonyl, wherein the eleven last-mentioned radicals may be unsubstituted, partially or fully halogenated and carry 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, wherein the phenyl is unsubstituted, or partially or fully halogenated and carry 0, 1, 2 or 3 substituents, independently of one another selected from the group consisting of C₁-C₄-alkyl, C₁-C₄-haloalkyl, C₁-C₄-alkoxy and C₁-C₄-haloalkoxy, and wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl, aryl-C₁-C₆-alkyl, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂, and N—Rⁿ, wherein Rⁿ is hydrogen, C₁-C₆-alkyl, or (C₁-C₆-alkyl)-carbonyl;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, (C₁-C₄-alkoxy)carbonyl, amino, (C₁-C₄-alkyl)amino, di(C₁-C₄-alkyl)amino, aminocarbonyl, (C₁-C₄-alkyl)aminocarbonyl, di(C₁-C₄-alkyl)aminocarbonyl, sulfonyl, sulfonylamino, sulfenylamino, sulfanylamino and C(═O)—R^{2a}, C(═O)R^{3a}, or C(═O)—R^{4a}, wherein each of R^{2a}, R^{3a}, and R^{4a} is selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ or N—Rⁿ, wherein Rⁿ is hydrogen, C₁-C₆-alkyl or (C₁-C₆-alkyl)-carbonyl;
R⁵ is selected from the group consisting of OR^{5a}, C₁-C₁₀-alkyl, C₂-C₁₀-alkenyl, C₂-C₁₀-alkinyl, and C₃-C₁₀-cycloalkyl, wherein each radical is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy, and C₁-C₆-haloalkoxy, wherein
R^{5a} is selected from the group consisting of hydrogen, C₁-C₁₀-alkyl, C₁-C₁₀-acyl, C₃-C₁₀-cycloalkyl, C₂-C₁₀-alkenyl, C₂-C₁₀-alkynyl, aryl, aryl-C₁-C₄-alkyl, heteroaryl, heteroaryl-C₁-C₄-alkyl, heterocyclyl and heterocyclyl-C₁-C₄-alkyl and wherein one or more carbon atoms of the radicals is unsubstituted, or partially or fully halogenated and carries 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
R⁶ is selected from the group consisting of C₁-C₁₀-alkyl, C₂-C₁₀-alkenyl, C₂-C₁₀-alkinyl, and C₃-C₁₀-cycloalkyl, wherein each of the four last-mentioned radicals is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy and C₁-C₆-haloalkoxy, or
R⁵/R^{5a} and R⁶ can join to form an unsaturated, or partially or fully saturated 3-10 membered ring, unsubstituted or substituted with an optionally substituted C₁-C₅-alkyl, wherein one or more carbon ring atoms are replaced by 0 to 3 heteroatoms selected from the group consisting of N, NRⁿ, O, S, SO, and SO₂ wherein Rⁿ is hydrogen, C₁-C₆-alkyl or (C₁-C₆-alkyl)-carbonyl;
or the enantiomers or diastereomers, salts or esters thereof.

28. A method for the protection of seeds from soil insects or the protection of the seedlings' roots and shoots from soil and foliar insects comprising contacting the seeds before sowing or after pregermination, or both, with at least one compound of the formula I or a composition comprising at least one compound of formula I: 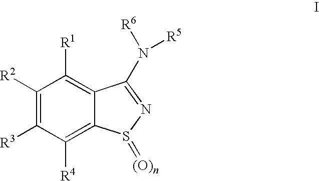
wherein
n is 0, 1 or 2;
R¹ is hydrogen, nitro, cyano, azido, amino, halogen, sulfonylamino, sulfenylamino, sulfinylamino, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, C₁-C₆-alkylthio, (C₁-C₆-alkyl)amino, di(C₁-C₆-alkyl)amino, C₁-C₆-alkylsulfinyl or C₁-C₆-alkylsulfonyl, wherein the eleven last-mentioned radicals may be unsubstituted, partially or fully halogenated and carry 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, wherein the phenyl is unsubstituted, or partially or fully halogenated and carry 0, 1, 2 or 3 substituents, independently of one another selected from the group consisting of C₁-C₄-alkyl, C₁-C₄-haloalkyl, C₁-C₄-alkoxy and C₁-C₄-haloalkoxy, and wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl, aryl-C₁-C₆-alkyl, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂, and N—Rⁿ, wherein Rⁿ is hydrogen, C₁-C₆-alkyl, or (C₁-C₆-alkyl)-carbonyl;
R², R¹ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, (C₁-C₄-alkoxy)carbonyl, amino, (C₁-C₄-alkyl)amino, di(C₁-C₄-alkyl)amino, aminocarbonyl, (C₁-C₄-alkyl)aminocarbonyl, di(C₁-C₄-alkyl)aminocarbonyl, sulfonyl, sulfonylamino, sulfenylamino, sulfanylamino and C(═O)—R^{2a}, C(═O)—R^{3a}, or C(═O)R^{4a}, wherein each of R^{2a}, R^{3a}, and R^{4a} is selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ or N—Rⁿ, wherein Rⁿ is hydrogen, C₁-C₆-alkyl or (C₁-C₆-alkyl)-carbonyl;
R⁵ is selected from the group consisting of OR^{5a}, C₁-C₁₀-alkyl, C₂-C₁₀-alkenyl, C₂-C₁₀-alkinyl, and C₃-C₁₀-cycloalkyl, wherein each radical is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy, and C₁-C₆-haloalkoxy, wherein
R^{5a} is selected from the group consisting of hydrogen, C₁-C₁₀-alkyl, C₁-C₁₀-acyl, C₃-C₁₀-cycloalkyl, C₂-C₁₀-alkenyl, C₂-C₁₀-alkynyl, aryl, aryl-C₁-C₄-alkyl, heteroaryl, heteroaryl-C₁-C₄-alkyl, heterocyclyl and heterocyclyl-C₁-C₄-alkyl and wherein one or more carbon atoms of the radicals is unsubstituted, or partially or fully halogenated and carries 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
R⁶ is selected from the group consisting of C₁-C₁₀-alkyl, C₂-C₁₀-alkenyl, C₂-C₁₀-alkinyl, and C₃-C₁₀-cycloalkyl, wherein each of the four last-mentioned radicals is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy and C₁-C₆-haloalkoxy, or
R⁵/R^{5a} and R⁶ can join to form an unsaturated, or partially or fully saturated 3-10 membered ring, unsubstituted or substituted with an optionally substituted C₁-C₅-alkyl, wherein one or more carbon ring atoms are replaced by 0 to 3 heteroatoms selected from the group consisting of N, NRⁿ, O, S, SO, and SO₂ wherein
Rⁿ is hydrogen, C₁-C₆-alkyl or (C₁-C₆-alkyl)-carbonyl;
or the enantiomers or diastereomers, salts or esters thereof.

29. The method of claim 26, wherein n is 2.

30. The method of claim 26, wherein n is 0.

31. The method of claim 26, wherein
R¹ is halogen, C(═O)R^{1a}, C₁-C₆-alkyl, C₂-C₆-alkenyl, C₂-C₆-alkynyl, C₃-C₈-cycloalkyl, C₁-C₆-alkoxy, or C₁-C₆-haloalkoxy, wherein the radicals may be unsubstituted, partially or fully halogenated and carry 0, 1, 2 or 3 radicals, selected from the group consisting of C₁-C₄-alkoxy, C₁-C₄-haloalkoxy, (C₁-C₄-alkoxy)carbonyl and C₃-C₈-cycloalkyl, and wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl and aryl-C₁-C₆-alkyl.

32. The method of claim 26, wherein
R¹ is halogen, C₁-C₆-alkoxy, C₁-C₆-haloalkoxy, wherein the radicals may be unsubstituted, partially or fully halogenated and carry 0, 1, 2 or 3 radicals, selected from the group consisting of C₁-C₄-alkoxy and C₁-C₄-haloalkoxy.

33. The method of claim 27, wherein n is 2.

34. The method of claim 27, wherein n is 0.

35. The method of claim 27, wherein
R¹ is halogen, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy, C₁-C₄-haloalkoxy, (C₁-C₄-alkoxy)carbonyl and C₃-C₈-cycloalkyl, and wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl and aryl-C₁-C₆-alkyl.

36. The method of claim 27, wherein
R¹ is halogen, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy and C₁-C₄-haloalkoxy.

37. The method of claim 28, wherein n is 2.

38. The method of claim 28, wherein n is 0.

39. The method of claim 28, wherein
R¹ is halogen, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy, C₁-C₄-haloalkoxy, (C₁-C₄-alkoxy)carbonyl and C₃-C₈-cycloalkyl, and wherein,
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl and aryl-C₁-C₆-alkyl.

40. The method of claim 28, wherein
R¹ is halogen, C₁-C₆-alkoxy, C₁-C₆-haloalkoxy, wherein the radicals may be unsubstituted, partially or fully halogenated and/or may carry 1, 2 or 3 radicals, selected from the group consisting of C₁-C₄-alkoxy and C₁-C₄-haloalkoxy.

41. The method of claim to 28, wherein the compound of formula I is applied in an amount of from 0.1 g to 10 kg per 100 kg of seeds.

42. The method of claim 41, wherein of the resulting plant's roots and shoots are protected.

43. The method of claim 41, wherein the resulting plant's shoots are protected from aphids.

44. Seed comprising a compound of formula I 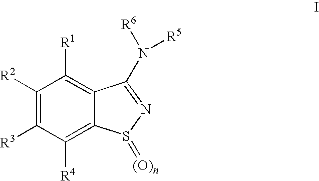
wherein
n is 0, 1 or 2;
R¹ is hydrogen, nitro, cyano, azido, amino, halogen, sulfonylamino, sulfenylamino, sulfinylamino, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, C₁-C₆-alkylthio, (C₁-C₆-alkyl)amino, di(C₁-C₆-alkyl)amino, C₁-C₆-alkylsulfinyl or C₁-C₆-alkylsulfonyl, wherein the eleven last-mentioned radicals may be unsubstituted, partially or fully halogenated and carry 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, wherein the phenyl is unsubstituted, or partially or fully halogenated and carry 0, 1, 2 or 3 substituents, independently of one another selected from the group consisting of C₁-C₄-alkyl, C₁-C₄-haloalkyl, C₁-C₄-alkoxy and C₁-C₄-haloalkoxy, and wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl, aryl-C₁-C₆-alkyl, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂, and N—Rⁿ, wherein Rⁿ is hydrogen, C₁-C₆-alkyl, or (C₁-C₆-alkyl)-carbonyl;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, (C₁-C₄-alkoxy)carbonyl, amino, (C₁-C₄-alkyl)amino, di(C₁-C₄-alkyl)amino, aminocarbonyl, (C₁-C₄-alkyl)aminocarbonyl, di(C₁-C₄-alkyl)aminocarbonyl, sulfonyl, sulfonylamino, sulfenylamino, sulfanylamino and C(═O)—R^{2a}, C(═O)—R^{3a}, or C(═O)—R^{4a}, wherein each of R^{2a}, R^{3a}, and R^{4a} is selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ or N—Rⁿ, wherein Rⁿ is hydrogen, C₁-C₆-alkyl or (C₁-C₆-alkyl)-carbonyl;
R⁵ is selected from the group consisting of OR^{5a}, C₁-C₁₀-alkyl, C₂-C₁₀-alkenyl, C₂-C₁₀-alkinyl, and C₃-C₁₀-cycloalkyl, wherein each radical is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy, and C₁-C₆-haloalkoxy, wherein
R^{5a} is selected from the group consisting of hydrogen, C₁-C₁₀-alkyl, C₁-C₁₀-acyl, C₃-C₁₀-cycloalkyl, C₂-C₁₀-alkenyl, C₂-C₁₀-alkynyl, aryl, aryl-C₁-C₄-alkyl, heteroaryl, heteroaryl-C₁-C₄-alkyl, heterocyclyl and heterocyclyl-C₁-C₄-alkyl and wherein one or more carbon atoms of the radicals is unsubstituted, or partially or fully halogenated and carries 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
R⁶ is selected from the group consisting of C₁-C₁₀-alkyl, C₂-C₁₀-alkenyl, C₂-C₁₀-alkinyl, and C₃-C₁₀-cycloalkyl, wherein each of the four last-mentioned radicals is unsubstituted, or partially or fully halogenated and carries 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy and C₁-C₆-haloalkoxy, or
R⁵/R^{5a} and R⁶ can join to form an unsaturated, or partially or fully saturated 3-10 membered ring, unsubstituted or substituted with an optionally substituted C₁-C₅-alkyl, wherein one or more carbon ring atoms are replaced by 0 to 3 heteroatoms selected from the group consisting of N, NRⁿ, O, S, SO, and SO₂ wherein
Rⁿ is hydrogen, C₁-C₆-alkyl or (C₁-C₆-alkyl)-carbonyl;
or an agriculturally useful salt thereof, in an amount of from 0.1 g to 10 kg per 100 kg of seed.

45. A compound of formula I 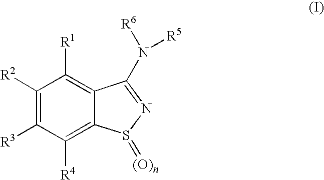
wherein
n is 0, 1 or 2;
R¹ is nitro, cyano, azido, amino, halogen, sulfonylamino, sulfenylamino, sulfinylamino, C(═O)R^{1a}, C₁-C₆-alkyl, C₂-C₆-alkenyl, C₂-C₆-alkynyl, C₃-C₈-cycloalkyl, C₁-C₆-alkoxy, C₁-C₆-haloalkoxy, C₁-C₆-alkylthio, (C₁-C₆-alkyl)amino, di(C₁-C₆-alkyl)amino, C₁-C₆-alkylsulfinyl or C₁-C₆-alkylsulfonyl, wherein the eleven last-mentioned radicals may be unsubstituted, or partially or fully halogenated and carry 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, wherein the phenyl is unsubstituted, or partially or fully halogenated and carries 0, 1, 2 or 3 substituents, independently of one another selected from the group consisting of C₁-C₄-alkyl, C₁-C₄-haloalkyl, C₁-C₄-alkoxy and C₁-C₄-haloalkoxy and
wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl, aryl-C₁-C₆-alkyl, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ and N—Rⁿ, wherein Rⁿ is hydrogen, C₁-C₆-alkyl, or (C₁-C₆-alkyl)-carbonyl;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, (C₁-C₄-alkoxy)carbonyl, amino, (C₁-C₄-alkyl)amino, di(C₁-C₄-alkyl)amino, aminocarbonyl, (C₁-C₄-alkyl)aminocarbonyl, di(C₁-C₄-alkyl)aminocarbonyl, sulfonyl, sulfonylamino, sulfenylamino, sulfanylamino and C(═O)—R^{2a}, C(═O)—R^{3a} or C(═O)R^{4a}, and wherein,
R^{2a} or R^{3a} or R^{4a} are selected from the group consisting of hydrogen, hydroxy, C₁-C₆-alkoxy, amino, C₁-C₆-alkyl, aryl, aryl-C₁-C₆-alkyl, (C₁-C₆-alkyl)-amino, di-(C₁-C₆-alkyl)-amino, 3- to 7-membered heteroaryl and heteroaryl-C₁-C₄-alkyl, wherein the heteroaryl ring contains as ring members 1, 2 or 3 heteroatoms, selected from the group consisting of nitrogen, oxygen, sulfur, a group SO, SO₂ and NRⁿ, wherein Rⁿ is hydrogen, C₁-C₆-alkyl or (C₁-C₆-alkyl)-carbonyl;
R⁵ is selected from the group consisting of OR^{5a}, C₁-C₁-alkyl, C₂-C₁₀-alkenyl, C₂-C₁₀-alkinyl, and C₃-C₁₀-cycloalkyl, wherein the four last-mentioned radicals may be unsubstituted, or partially or fully halogenated and carry 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy, and C₁-C₆-haloalkoxy, and wherein
R^{5a} is selected from the group consisting of hydrogen, C₁-C₁₀-alkyl, C₁-C₁₀-acyl, C₃-C₁₀-cycloalkyl, C₂-C₁₀-alkenyl, C₂-C₁₀-alkynyl, aryl, aryl-C₁-C₄-alkyl, heteroaryl, heteroaryl-C₁-C₄-alkyl, heterocyclyl and heterocyclyl-C₁-C₄-alkyl and wherein one or morecarbon atoms of the radicals may be unsubstituted, or partially or fully halogenated and carry 0, 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
R⁶ is selected from the group consisting of C₁-C₁₀-alkyl, C₂-C₁₀-alkenyl, C₂-C₁₀-alkinyl, and C₃-C₁₀-cycloalkyl, wherein the four last-mentioned radicals may be unsubstituted, or partially or fully halogenated and carry 0-4 radicals selected from the group consisting of C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, (C₁-C₁₀-alkoxy)carbonyl, cyano, nitro, amino, (C₁-C₁₀-alkyl)amino, di-(C₁-C₁₀-alkyl)amino, C₃-C₁₀-cycloalkyl and phenyl, wherein the phenyl is unsubstituted, or partially or fully halogenated and carries 0-3 substituents selected from the group consisting of C₁-C₆-alkyl, C₁-C₆-haloalkyl, C₁-C₆-alkoxy and C₁-C₆-haloalkoxy;
and wherein the compound of formula I does not represent 4-chloro-3-N,N-dimethyl-amino-1,2 benzisothiazol.

46. The compound of claim 45, wherein n is 2.

47. The compound of claim 45, wherein n is 0.

48. The compound of claim 45, wherein
R¹ is halogen, C(═O)R^{1a}, C₁-C₆-alkyl, C₂-C₆-alkenyl, C₂-C₆-alkynyl, C₃-C₈-cycloalkyl, C₁-C₆-alkoxy, or C₁-C₆-haloalkoxy, wherein the six last-mentioned radicals are unsubstituted, or partially or fully halogenated and carry 0, 1, 2 or 3 radicals, selected from the group consisting of C₁-C₄-alkoxy, C₁-C₄-haloalkoxy, (C₁-C₄-alkoxy)carbonyl and C₃-C₈-cycloalkyl, and wherein
R^{1a} is selected from the group consisting of hydrogen, hydroxy, C₁-C₆-alkoxy, amino, (C₁-C₆-alkyl)amino, di(C₁-C₆-alkyl)amino, C₁-C₆-alkyl, aryl and aryl-C₁-C₆-alkyl.

49. The compound of claim 45, wherein
R¹ is halogen, C₁-C₆-alkoxy, or C₁-C₆-haloalkoxy, wherein the two last-mentioned radicals are unsubstituted, or partially or fully halogenated and carry 0, 1, 2 or 3 radicals, selected from the group consisting of C₁-C₄-alkoxy and C₁-C₄-haloalkoxy.
